SEQUENCE LISTING (34 sequences)

SEQ ID NO: 1 - DNA, 253 bp, Chlamydomonas reinhardtii
cgcttagaagatttcgataaggcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaacggtcttaagaagtctatccgg

SEQ ID NO: 2 - DNA, 312 bp, Chlamydomonas reinhardtii
ctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaac

SEQ ID NO: 3 - DNA, 356 bp, Chlorella virus
cggggatcgcagggcatgggcattaaaagaactttatggaatcaaaaatcttagtgaatttccaccacaggtatatagtcttcaggacgctaacgatgatatcaacgattgtatcaaaggttatcgtttgaggcactcatatcaggtagtttctacacagaaacttgaacaacgcctgggaaaagatcctgagcatagtaacttatatactagcagatgttgtaacgatgctttatatgaatatgaattagcacaacgacaactacaaaaacaacttgatgaatttgacgaagatgggtatgatttttttcaggcacgtataaatacattagatccgtcgacctgcagccaagctt

SEQ ID NO: 4 - DNA, 207 bp, Chlorella virus
cccggggatcatcgaaagcaactgccgcattcgaaacttcgactgcctcgttataaaggttagtgaaagccattgtatgttattactgagttatttaatttagcttgcttaaatgcttatcgtgttgatatgataaatgacaaatgatacgctgtatcaacatctcaaaagattaatacgaagatccgtcgacctgcagccaagctt

SEQ ID NO: 5 - DNA, 277 bp, Chlorella virus
cccggggatctgcgtattgcgggacttttgagcattttccagaacggattgccgggacgtatactgaacctccagtccctttgctcgtcgtatttcccataatatacatatacactattttaattatttacaccggttgttgctgagtgatacaatgcaaattccctccaccgaggaggatcgcgaactgtccaaatgtcttctttctgcagctccatacggagtcgttaggaaacattcacttaattataggatccgtcgacctgcagccaagctt

SEQ ID NO: 6 - DNA, 489 bp, Rhodella reticulata
tttttatagatcatccaattattttttcattagatattgtatatcaataatttggcatatgttttgtagtatacgggttatgatattgcaatatatgtacaacattggtaatttttggacttacatatatatcaattatatcaatgacaatgtaatatattggttgatagatcaataaacatctttaataagatctgttaaaattcaaatatagactttctgtattataagtagttttcttatattactatagacgtagaacgatcaaaaaaaaataaatatggacatgacttgattcaatatggaagacggggtatgagaaatatcgtgttgcactcaatatagaattgacgtatttttaatgcagtgcccgttatatattgcgtaacaaagattaaaagtatattatatattataatactagtagaccagcaaatataaaattatgctgaaacaataataccctttaaagttttaaggagccttttc

SEQ ID NO: 7 - DNA, 543 bp, Porphyridium sp.
attatttaacaattggaaacttagttaattagggtaaattatattaacccttatgaaccaaaataatttggtttcaaaaaaaactaacttatgaattaaaattgaaatattttctacatcataataattttaattctaaatagaattttagataagggatctaagataacaaaaaaatcaatttaagtaataaagaaaatgtgattacaaaatttttgatattaaactatagtatttacaaattattatcaaaaattacttatccatttgaggaaaagactgaacctttaaacatatttgtttatgcgattttagatcattcaagttagcgagctgtatgaaatgaaagtttcatgtacagttcttaagtagagatgtatatatgttaatagaaatattatttgcatcgactataatcaattctgaagacttcaaaataaaacctgttatacgtgctatactagagatggttgatgaaataaatcaaccaggtattattacagactgaactgaactaaaaaaattcatataatttagcgtact

SEQ ID NO: 8 - DNA, 799 bp, Porphyridium purpureum
gcacacgagtgttgtggcgttgtcgcagcaggtttgggggcgcgagagcgcacgacgcttgtgtgtgtgtgtgtgtgtggaccgcaaccaccctcgcgacgcggcattgccgtgcgtgccgtcgcggctgcgtggttcgtggtgtgatattctaaacgcatgtgggttgggtgtgggtgttgttctgtgtccatcaggcgatggacacagccgccactgaagtgtcactgaattaagcgcggtgcattttgcacgtggcttttgtgtgggtgtgtgtgtatgtgtcctgctcggcttgtatcgacatcctccttcgtttttctcgtacggggcttttgtgtttcctttggtacgtggtgagcgttttttggggtgttgccggacatgatggtgttgtgtttgtgagtttgggagtgtgagactgggagcgacggtgaagccgcatgaatcgtggagcgcaaaatgcaagttgactggagccatcgcgatgcttttggcgttttgcgcatgtgatcacaatctcctcggaatggtccaaaatggatcgaactggctcgccccccaatctgtgcgctttcggcctgttcggacatgccggtttcgcggtgcgcagcatgtggctcgcgcatggtaggggatgttggcgcggggcataaataggctgcgacaacttgccgcttccccttcatcgcacacctcaggcaggaggaagtggtggaaaagactggtgcaggagaggattttgcaggagaggaaggagagggagaggcgtgtcgtgcttgccactgcgatagtcacc

SEQ ID NO: 9 - DNA, 848 bp, Porphyridium purpureum
gcgtgcgtcaagcacattggggcaactcgggcaaccgacgcagccacgcacacgagtgttgtggcgttgtcgtagcaggtttgggggcgcgagagcgcacgacgcgtgtgtgtgtgtgtgtgtgtggaccgcaaccaccctcgcgacgcggcattgccgtgcctgccgttgcggctgcgtggttcgtggtgtgatattctaaacgcatgtgggttgggtgttggtgttgttctgtgtccatcaggcgatggacacagccgccactgaagtgtcactgaattaagcgcggtgcattttgcacgtggcttttgtgtgtgtgtgtttgtgtctatgtgtcctgctcggtttgtatcgacgtcctccttcgtttttttcgcacggggcttttgtctttcctttggtacgtggtgagcgttttttggggtgttgccggacatgatggtgttgtgtttgtgagtttgagagtgagactgggagcgacggtgaagccgcatgaatcgtggagcgcaaaatgcaagttgactggagccatcgcgatgcttttggcgttttgcgcatgtgatcacaatctcctcggaatggtccaaaatggatcgaactggctcgccccccaatctgtgcgctttcggcctgttcggacatgccggtttcgtggtgcgcagcatgtggctcgcgcatggtaggggatgttggcgcggggcataaataggctgcgacaacttgccgcttccccttccctgcacgcctcaggcaggaagaagtggtggaaaagactggtgcaggagaggatcttgcaggagaggaaggagagggagaggcgtgtcgtgcttgccactgcaatcgtcacc

SEQ ID NO: 10 - protein, 587 aa, Porphyridium sp.
MTHIEKSNYQEQTGAFALLDSLVRHKVKHIFGYPGGAILPIYDELYKWEEQGYIKHILVRHEQGAAHAADGYARATGEVGVCFATSGPGATNLVTGIATAHMDSIPIVIITGQVGRSFIGTDAFQEVDIFGITLPIVKHSYVIRDPRDIPRIVAEAFSIAKQGRPGPVLIDVPKDVGLETFEYQYVNPGEARIPGFRDLVAPSSRQIIHSIQLIQEANQPLLYVGGGAITSGAHDLIYKLVNQYKIPITTTLMGKGIIDEQNPLALGMLGMHGTAYANFAVSECDLLITLGARFDDRVTGKLDEFACNAKVIHVDIDPAEVGKNRIPQVAIVGDISLVLEQWLLYLDRNLQLDDSHLRSWHERIFRWRQEYPLIVPKLVQTLSPQEIIANISQIMPDAYFSTDVGQHQMWAAQFVKTLPRRWLSSSGLGTMGYGLPAAIGAKIAYPESPVVCITGDSSFQMNIQELGTIAQYKLDIKIIIINNKWQGMVRQSQQAFYGARYSHSRMEDGAPNFVALAKSFGIDGQSISTRQEMDSLFNTIIKYKGPMVIDCKVIEDENCYPMVAPGKSNAQMIGLDKSNNEIIKIKE

SEQ ID NO: 11 - protein, 129 aa, Streptoalloteichus hindustanus
MARMAKLTSAVPVLTARDVAGAVEFWTDRLGFSRDFVEDDFAGVVRDDVTLFISAVQDQDQVVPDNTLAWVWVRGLDELYAEWSEVVSTNFRDASGPAMTEIGEQPWGREFALRDPAGNCVHFVAEEQD

SEQ ID NO: 12 - protein, 791 aa, Artificial sequence
  Synthetic construct
MARMVVAAVAVMAVLSVALAQFIPDVDITWKVPMTLTVQNLSIFTGPNQFGRGIPSPSAIGGGNGLDIVGGGGSLYISPTGGQVQYSRGSNNFGNQVAFTRVRKNGNNESDFATVFVGGTTPSFVIVGDSTENEVSFWTNNKVVVNSQGFIPPNGNSAGGNSQYTFVNGITGTAGAPVGGTVIRQVSAWREIFNTAGNCVKSFGLVVRGTGNQGLVQGVEYDGYVAIDSNGSFAISGYSPAVNNAPGFGKNFAAARTGNFFAVSSESGVIVMSIPVDNAGCTLSFSVAYTITPGAGRVSGVSLAQDNEFYAAVGIPGAGPGEVRIYRLDGGGATTLVQTLSPPDDIPELPIVANQRFGEMVRFGANSETNYVAVGSPGYAAEGLALFYTAEPGLTPNDPDEGLLTLLAYSNSSEIPANGGLGEFMTASNCRQFVFGEPSVDSVVTFLASIGAYYEDYCTCERENIFDQGIMFPVPNFPGESPTTCRSSIYEFRFNCLMEGAPSICTYSERPTYEWTEEVVDPDNTPCELVSRIQRRLSQSNCFQDYVTLQVVMEVQLVQSGGGVVQPGKSLRLSCAASGFAFSSYAMHWVRQAPGKGLEWVAVISYDGSNKYYADSVKGRFTISRDNSKNTLYLQMNSLRAEDTAVYYCARDRSYYLDYWGQGTLVTVSSGGGGSGGGGSGGGGSTTLTQSPATLSLSPGERATLSCRASQSVRSNLAWYQQKPGQAPRPLIYDASTRATGIPDRFSGSGSGTDFTLTISRLEPEDFAVYYCQQRSNWPPTFGQGTKVEVK

SEQ ID NO: 13 - protein, 534 aa, Chlorella kessleri
MAGGAIVASGGASRSSEYQGGLTAYVLLVALVAACGGMLLGYDNGVTGGVASMEQFERKFFPDVYEKKQQIVETSPYCTYDNPKLQLFVSSLFLAGLISCIFSAWITRNWGRKASMGIGGIFFIAAGGLVNAFAQDIAMLIVGRVLLGFGVGLGSQVVPQYLSEVAPFSHRGMLNIGYQLFVTIGILIAGLVNYGVRNWDNGWRLSLGLAAVPGLILLLGAIVLPESPNFLVEKGRTDQGRRILEKLRGTSHVEAEFADIVAAVEIARPITMRQSWRSLFTRRYMPQLLTSFVIQFFQQFTGINAIIFYVPVLFSSLGSASSAALLNTVVVGAVNVGSTMIAVLLSDKFGRRFLLIEGGITCCLAMLAAGITLGVEFGQYGTEDLPHPVSAGVLAVICIFIAGFAWSWGPMGWLIPSEIFTLETRPAGTAVAVMGNFLFSFVIGQAFVSMLCAMKFGVFLFFAGWLVIMVLCAIFLLPETKGVPIERVQALYARHWFWKKVMGPAAQEIIAEDEKRVAASQAIMKEERISQTMK

SEQ ID NO: 14 - DNA, 1605 bp, Artificial sequence
  Synthetic construct
atggcgggcggcgccattgttgccagcggcggcgccagccgttcgagcgagtaccagggcggcctgaccgcctacgttctgctcgtggcgctggttgccgcctgcggcggcatgctgctgggctacgacaacggcgttaccggcggcgttgccagcatggagcagttcgagcgcaagttcttcccggacgtgtacgagaagaagcagcagattgtcgagaccagcccgtactgcacctacgacaacccgaagctccagctgttcgtgtcgagcctgttcctggcgggcctgattagctgcattttctcggcgtggattacccgcaactggggccgcaaggcgagcatgggcattggcggcattttcttcattgccgccggtggcctggttaacgccttcgcccaggacattgccatgctgattgtgggccgcgtcctgctgggcttcggcgttggcctgggcagccaggtggtgccacagtacctgagcgaggtggcgccattcagccatcgcggcatgctcaacattggctaccagctcttcgtgaccattggcattctgattgccggcctggtgaactacggcgtgcgcaactgggacaacggttggcgcctgagcctgggcctggcggcggttccaggcctgattctgctgctcggcgccatcgttctgccggagagcccgaacttcctggtggagaagggccgcaccgaccagggccgccgcattctggagaagctgcgcggcaccagccatgttgaggcggagttcgccgacattgtggcggcggtggagattgcccgcccaattaccatgcgccagagctggcgctcgctgttcacccgccgctacatgccacagctgctgaccagcttcgtgattcagttcttccagcagttcaccggcattaacgccatcattttctacgtgccggtgctgttcagcagcctgggctcggcgtcctcggcggcgctgctgaacaccgtggttgtgggcgccgtgaacgtgggcagcaccatgattgccgtgctgctgtcggacaagttcggccgccgcttcctgctgattgagggcggcattacctgctgcctggcgatgctggcggcgggcattacgctgggcgtggagttcggccagtacggcaccgaggacctgccacatccagtgtcggcgggcgtgctggcggtgatttgcattttcattgccggcttcgcctggagctggggcccaatgggctggctgattccgagcgagattttcaccctggagacccgcccagcgggcacggcggttgccgtgatgggcaacttcctgttctcgttcgtgattggccaggccttcgtgtcgatgctgtgcgcgatgaagttcggcgtgttcctgttcttcgccggctggctggtgattatggtgctgtgcgccattttcctgctgccggagaccaagggcgtgccgattgagcgcgtgcaggcgctgtacgcccgccactggttctggaagaaggtgatgggcccagcggcccaggagattattgccgaggacgagaagcgcgttgcggcgagccaggcgattatgaaggaggagcgcattagccagaccatgaagtaa

SEQ ID NO: 15 - protein, 541 aa, Saccharomyces cerevisiae
MSEFATSRVESGSQQTSIHSTPIVQKLETDESPIQTKSEYTNAELPAKPIAAYWTVICLCLMIAFGGFVFGWDTGTISGFVNQTDFKRRFGQMKSDGTYYLSDVRTGLIVGIFNIGCAFGGLTLGRLGDMYGRRIGLMCVVLVYIVGIVIQIASSDKWYQYFIGRIISGMGVGGIAVLSPTLISETAPKHIRGTCVSFYQLMITLGIFLGYCTNYGTKDYSNSVQWRVPLGLNFAFAIFMIAGMLMVPESPRFLVEKGRYEDAKRSLAKSNKVTIEDPSIVAEMDTIMANVETERLAGNASWGELFSNKGAILPRVIMGIMIQSLQQLTGNNYFFYYGTTIFNAVGMKDSFQTSIVLGIVNFASTFVALYTVDKFGRRKCLLGGSASMAICFVIFSTVGVTSLYPNGKDQPSSKAAGNVMIVFTCLFIFFFAISWAPIAYVIVAESYPLRVKNRAMAIAVGANWIWGFLIGFFTPFITSAIGFSYGYVFMGCLVFSFFYVFFFVCETKGLTLEEVNEMYVEGVKPWKSGSWISKEKRVSEE

SEQ ID NO: 16 - DNA, 1626 bp, Artificial sequence
  Synthetic construct
atgagcgagttcgccacctcgcgcgttgagagcggcagccagcagaccagcattcacagcaccccgattgtccagaagctggagaccgacgagagcccgattcagaccaagagcgagtacaccaacgccgagctgccggcgaagccaattgccgcctactggaccgtgatttgcctgtgcctgatgattgccttcggcggcttcgtgttcggctgggacaccggcaccatttcgggcttcgtgaaccagaccgacttcaagcgccgcttcggccagatgaagagcgacggcacctactacctgagcgacgtgcgcaccggcctgattgtgggcattttcaacattggctgcgccttcggtggcctgaccctgggccgcctgggcgacatgtacggccgccgcattggcctgatgtgcgtggtgctggtgtacattgtcggcatcgtgattcagattgccagcagcgacaagtggtatcagtacttcattggccgcattattagcggcatgggcgtgggcggcattgccgttctgagcccgaccctgattagcgagaccgccccgaagcatattcgcggcacctgcgtgtcgttctaccagctgatgattaccctgggcatcttcctgggctactgcaccaactacggcaccaaggactacagcaacagcgtccagtggcgcgttccactgggcctgaacttcgccttcgccattttcatgattgccggcatgctgatggtgccagagagcccacgcttcctggttgagaagggccgctacgaggacgccaagcgctcgctggcgaagagcaacaaggtgaccattgaggacccgagcattgtggcggagatggacaccattatggcgaacgtggagaccgagcgcctggcgggcaacgccagctggggcgagctgttcagcaacaagggcgccattctgccgcgcgtgattatgggcattatgatccagagcctccagcagctgaccggcaacaactacttcttctactacggcacgaccattttcaacgccgtgggcatgaaggacagcttccagacctcgattgtgctgggcattgtcaacttcgccagcaccttcgtggcgctgtacaccgtggacaagttcggccgccgcaagtgcctgctgggcggctcggcgagcatggcgatttgcttcgtgattttcagcaccgtgggcgtgaccagcctgtacccgaacggcaaggaccagccgagcagcaaggcggccggcaacgtgatgattgtgttcacctgcctgttcatcttcttcttcgccattagctgggcgccgattgcctacgtgatcgtggcggagagctacccactgcgcgtgaagaaccgcgcgatggcgattgccgttggcgccaactggatttggggcttcctgattggcttcttcaccccgttcattacctcggcgattggcttcagctacggctacgtgttcatgggctgcctggtgttctcgttcttctacgtgttcttcttcgtgtgcgagaccaagggcctgacgctggaggaggtgaacgagatgtacgtggagggcgtgaagccgtggaagagcggctcgtggattagcaaggagaagcgcgtttcggaggagtaa

SEQ ID NO: 17 - protein, 492 aa, Homo sapiens
MEPSSKKLTGRLMLAVGGAVLGSLQFGYNTGVINAPQKVIEEFYNQTWVHRYGESILPTTLTTLWSLSVAIFSVGGMIGSFSVGLFVNRFGRRNSMLMMNLLAFVSAVLMGFSKLGKSFEMLILGRFIIGVYCGLTTGFVPMYVGEVSPTAFRGALGTLHQLGIVVGILIAQVFGLDSIMGNKDLWPLLLSIIFIPALLQCIVLPFCPESPRFLLINRNEENRAKSVLKKLRGTADVTHDLQEMKEESRQMMREKKVTILELFRSPAYRQPILIAVVLQLSQQLSGINAVFYYSTSIFEKAGVQQPVYATIGSGIVNTAFTVVSLFVVERAGRRTLHLIGLAGMAGCAILMTIALALLEQLPWMSYLSIVAIFGFVAFFEVGPGPIPWFIVAELFSQGPRPAAIAVAGFSNWTSNFIVGMCFQYVEQLCGPYVFIIFTVLLVLFFIFTYFKVPETKGRTFDEIASGFRQGGASQSDKTPEELFHPLGADSQV

SEQ ID NO: 18 - DNA, 1479 bp, Artificial sequence
  Synthetic construct
atggagccgagcagcaagaagctgaccggccgcctgatgctggcggttggcggcgccgttctgggcagcctccagttcggctacaacaccggcgtgattaacgccccacagaaggtgatcgaggagttctacaaccagacctgggtccaccgctacggcgagagcattctgccgaccaccctgaccacgctgtggagcctgagcgtggcgattttcagcgtgggcggcatgattggcagcttctcggtgggcctgttcgtgaaccgcttcggccgccgcaacagcatgctgatgatgaacctgctggccttcgtgtcggcggtgctgatgggcttcagcaagctgggcaagagcttcgagatgctgattctgggccgcttcattattggcgtgtactgcggcctgaccaccggcttcgtgccgatgtacgtgggcgaggtgtcgccaacggcgttccgcggcgcgctgggcaccctccatcagctgggcattgttgtgggcattctgattgcccaggtgttcggcctggacagcattatgggcaacaaggacctgtggccgctgctgctgtcgattattttcattccggcgctgctccagtgcattgtgctgccgttctgcccagagagcccacgcttcctgctgattaaccgcaacgaggagaaccgcgcgaagagcgtgctgaagaagctgcgcggcacggcggacgttacccacgacctccaggagatgaaggaggagagccgccagatgatgcgcgagaagaaggtgaccattctggagctgttccgctcgccagcgtaccgccagccgattctgatcgccgtggtgctccagctgtcccagcagctgtcgggcattaacgccgtgttctactacagcaccagcattttcgagaaggcgggcgtccagcagccagtgtacgccaccattggcagcggcattgtgaacaccgccttcaccgtggtgtcgctgttcgtggttgagcgcgcgggccgccgcacgctccatctgattggcctggcgggcatggcgggctgcgcgattctgatgaccattgccctggcgctgctggagcagctgccgtggatgagctacctgagcattgtggcgatcttcggcttcgtggcgttcttcgaggttggcccaggcccgattccgtggttcattgtggcggagctgttcagccagggcccacgcccagcggcgattgccgttgccggcttctcgaactggaccagcaacttcattgtgggcatgtgcttccagtacgtcgagcagctgtgcggcccgtacgtgttcattatcttcaccgtgctgctggtcctcttcttcatcttcacctacttcaaggtgccggagaccaagggccgcaccttcgacgagattgccagcggcttccgccagggcggcgccagccagagcgacaagaccccggaggagctgttccatccactgggcgccgacagccaggtgtaa

SEQ ID NO: 19 - protein, 1039 aa, Artificial sequence
  Synthetic construct
MQAKASTSPLGDSIEPRTENLEYATEQKESFVPRRAFGTAAERARRNLNAKLANPLSGYSHEELRRQGINFAITHQIGDEGDIRAFGLGAMLAQAPEKFENVPGLTVQELEVLRHEFEHRWSQPWTMYLVIILCSLSAAVQGMDETVVNGAQIFYKHQFGIADENISRHNWISGLVNAAPYLCCAIVGCWLTVPFNSWFGRRGTIFITCIFSATTCLWQGCCSTWWSLFIARFALGFGIGPKSATVPVYAAETGGLLLELCLVPDSSGIVGLNWRLMLASALVPAVIVCCFVFMCPESPRWYMSRNLYDRAYQSMCSLRFNKVQAARDMYYMYTLLEAEKSMKLGQNKLLELINVPRNRRAMFASEIVMFMQQFCGVNVLAYYSSEIFLQTASEHSKLTVSNQRKALTASLGWGLINWLFAIPAVYTIDTFGRRNLLLSTFPLMALSMFGPPSSFFFFFFFTKWVNFGLFLVAVFIFIAAYSPANGPVPWVYCPEIFPLYVRAQGMAITTFFNYLFNFVVSYSWPDMLQKLKAQGGYGFYAGAIAVGWVLLFFFMPETKGYTLEQMGMVFEHSLGEIARYHWKCGIRNIRKLFGLPTSSEPLASPYNKKLNLKMHGVEERVIQRQRLLPQQQRRNQSKSELPDGKPSVVSVILGLNAIESREIAQIIFYNAKMDASENQAQAQQQTPQKPTYQNGVRTNGRAFNSPNWRVKREESPSGSRSPSQDTQNGSPRRTPGFGRQNREVPQAISEGRRLYVGNMPYTAKMEDVQELFTRGGFEVVRIDISIDPFSGRNPSYCFVDLSTKELAERAMAELDGGDLLGRPVRIKPGVVKSASERQPQQRTGMGAGTGSIGDGMSSGSPRANRAGSSPLNADRWRRDDNLTSASTTPTKLGNMSTYNPKADPSKRLYVGGLPRLTDPDAISSNITQFFKGYNLTNISKLFTPHPAKRFEPGDHYYLFVDFETVEETQNAMAALNGAEGPWGAAIRVQRARGETWKNTDSNNTSEERRPAAGRWGPTTRRQDVASTPAPASGEAAVQA

SEQ ID NO: 20 - protein, 661 aa, Artificial sequence
  Synthetic construct
MVEKSSDPEVPSLSHHESSISIEKQGDAATAREWAQDVNSTTTNTKLKNPLAGLTREQLLNDVEAFAKEKDLEHILDDLRKGALVAQDPREFEQMDALTESEKELLRREKTHRWSQPFMMYFMTSESSRYPPTEFGFNPACQSSVLDLLSCREWIRLLSTVRRSMYSSITHLSYAKQSRFYFAEFNVTDTWMQGLLNGAPYLCSAVIGCWTTAPLNRWFGRRGCIFISCFISFASSFWMAAAHTWWNLLLGRFLLGFAVGAKSTTTPVYGAECSPANIRGALVMMWQMWTAFGIMLGYIASVAFMDVTHPTIPGFNWRLMLGSTAIPPFFVCIQVYTVPESPRWLIKRRRYEDAKRNLFKLRRTAETAERDFVRIKKGVEEDEILQKGKNLLVEVIPVPYIRRALLIGIMEMLFQQMSGMNVFMNYIDEVFEENINMGARTSVAVSLFPGFVNMVATVIVYFTIDRYGRRTLQLVTFPVMFLMLLMVLFSFYGDKKVNLAFFIIGVVFFIVAYSPGAGPVPWTFCAEVFPTYVRAAGTTITTFFVNAFNFALSFSWPSMKAAWGPQGGFGFYAGFNFLGIVMQFLFLPETKGFTLEQMRVVFEEGLFTIAAYHCRAGWRSLRKLLGLSVPDTPLVSPYDKAFAIDRAKREEEMMHAGEVSK

SEQ ID NO: 21 - protein, 552 aa, Porphyridium sp.
MARMVVAAVAVMAVLSVALAQFIPDVDITWKVPMTLTVQNLSIFTGPNQFGRGIPSPSAIGGGNGLDIVGGGGSLYISPTGGQVQYSRGSNNFGNQVAFTRVRKNGNNESDFATVFVGGTTPSFVIVGDSTENEVSFWTNNKVVVNSQGFIPPNGNSAGGNSQYTFVNGITGTAGAPVGGTVIRQVSAWREIFNTAGNCVKSFGLVVRGTGNQGLVQGVEYDGYVAIDSNGSFAISGYSPAVNNAPGFGKNFAAARTGNFFAVSSESGVIVMSIPVDNAGCTLSFSVAYTITPGAGRVSGVSLAQDNEFYAAVGIPGAGPGEVRIYRLDGGGATTLVQTLSPPDDIPELPIVANQRFGEMVRFGANSETNYVAVGSPGYAAEGLALFYTAEPGLTPNDPDEGLLTLLAYSNSSEIPANGGLGEFMTASNCRQFVFGEPSVDSVVTFLASIGAYYEDYCTCERENIFDQGIMFPVPNFPGESPTTCRSSIYEFRFNCLMEGAPSICTYSERPTYEWTEEVVDPDNTPCELVSRIQRRLSQSNCFQDYVTLQVV

SEQ ID NO: 22 - protein, 523 aa, Nicotiana tabacum
MAGGGGIGPGNGKEYPGNLTLYVTVTCIVAAMGGLIFGYDIGISGGVTSMDSFLSRFFPSVFRKQKADDSTNQYCKFDSQTLTMFTSSLYLAALLSSLVASTVTRKLGRRLSMLCGGVLFCAGALINGFAQNVAMLIVGRILLGFGIGFANQSVPLYLSEMAPYKYRGALNLGFQLSITIGILVANVLNYFFAKIHWGWRLSLGGAMVPALIITIGSLFLPETPNSMIERGNHDEAKARLKRIRGIDDVDEEFNDLVVASEASRKIENPWRNLLQRKYRPHLTMAIMIPFFQQLTGINVIMFYAPVLFKTIGFGADASLMSAVITGGVNVLATVVSIYYVDKLGRRFLFLEGGIQMLICQIAVSICIAIKFGVNGTPGDLPKWYAIVVVIFICVYVAGFAWSWGPLGWLVPSEIFPLEIRSAAQSINVSVNMIFTFIVAQVFLTMLCHLKFGLFLFFAFFVVIMTVFIYFFLPETKNIPIEEMVIVWKEHWFWSKFMTEVDYPGTRNGTSVEMSKGSAGYKIV

SEQ ID NO: 23 - protein, 522 aa, Arabidopsis thaliana
MPAGGFVVGDGQKAYPGKLTPFVLFTCVVAAMGGLIFGYDIGISGGVTSMPSFLKRFFPSVYRKQQEDASTNQYCQYDSPTLTMFTSSLYLAALISSLVASTVTRKFGRRLSMLFGGILFCAGALINGFAKHVWMLIVGRILLGFGIGFANQAVPLYLSEMAPYKYRGALNIGFQLSITIGILVAEVLNYFFAKIKGGWGWRLSLGGAVVPALIITIGSLVLPDTPNSMIERGQHEEAKTKLRRIRGVDDVSQEFDDLVAASKESQSIEHPWRNLLRRKYRPHLTMAVMIPFFQQLTGINVIMFYAPVLFNTIGFTTDASLMSAVVTGSVNVGATLVSIYGVDRWGRRFLFLEGGTQMLICQAVVAACIGAKFGVDGTPGELPKWYAIVVVTFICIYVAGFAWSWGPLGWLVPSEIFPLEIRSAAQSITVSVNMIFTFIIAQIFLTMLCHLKFGLFLVFAFFVVVMSIFVYIFLPETKGIPIEEMGQVWRSHWYWSRFVEDGEYGNALEMGKNSNQAGTKHV

SEQ ID NO: 24 - protein, 516 aa, Vicia faba
MPAAGIPIGAGNKEYPGNLTPFVTITCVVAAMGGLIFGYDIGISGGVTSMNPFLEKFFPAVYRKKNAQHSKNQYCQYDSETLTLFTSSLYLAALLSSVVASTITRRFGRKLSMLFGGLLFLVGALINGLAQNVAMLIVGRILLGFGIGFANQSVPLYLSEMAPYKYRGALNIGFQLSITIGILVANILNYFFAKIKGGWGWRLSLGGAMVPALIITIGSLILPDTPNSMIERGDRDGAKAQLKRIRGVEDVDEEFNDLVAASETSMQVENPWRNLLQRKYRPQLTMAVLIPFFQQFTGINVIMFYAPVLFNSIGFKDDASLMSAVITGVVNVVATCVSIYGVDKWGRRALFLEGGVQMLICQVAVAVSIAAKFGTSGEPGDLPKWYAIVVVLFICIYVAGFAWSWGPLGWLVPSEIFPLEIRSAAQSVNVSVNMLFTFLVAQIFLTMLCHMKFGLFLFFAFFVVVMTIYIYTMLPETKGIPIEEMDRVWKSHPYWSRFVEHDDNGVEMAKGGVKNV

SEQ ID NO: 25 - protein, 540 aa, Parachlorella kessleri
MAGGGPVASTTTNRASQYGYARGGLNWYIFIVALTAGSGGLLFGYDIGVTGGVTSMPEFLQKFFPSIYDRTQQPSDSKDPYCTYDDQKLQLFTSSFFLAGMFVSFFAGSVVRRWGRKPTMLIASVLFLAGAGLNAGAQDLAMLVIGRVLLGFGVGGGNNAVPLYLSECAPPKYRGGLNMMFQLAVTIGIIVAQLVNYGTQTMNNGWRLSLGLAGVPAIILLIGSLLLPETPNSLIERGHRRRGRAVLARLRRTEAVDTEFEDICAAAEESTRYTLRQSWAALFSRQYSPMLIVTSLIAMLQQLTGINAIMFYVPVLFSSFGTARHAALLNTVIIGAVNVAATFVSIFSVDKFGRRGLFLEGGIQMFIGQVVTAAVLGVELNKYGTNLPSSTAAGVLVVICVYVAAFAWSWGPLGWLVPSEIQTLETRGAGMSMAVIVNFLFSFVIGQAFLSMMCAMRWGVFLFFAGWVVIMTFFVYFCLPETKGVPVETVPTMFARHWLWGRVMGEKGRALVAADEARKAGTVAFKVESGSEDGKPASDQ

SEQ ID NO: 26 - protein, 383 aa, Arabidopsis thaliana
MAVGSMNVEEGTKAFPAKLTGQVFLCCVIAAVGGLMFGYDIGISGGVTSMDTFLLDFFPHVYEKKHRVHENNYCKFDDQLLQLFTSSLYLAGIFASFISSYVSRAFGRKPTIMLASIFFLVGAILNLSAQELGMLIGGRILLGFGIGFGNQTVPLFISEIAPARYRGGLNVMFQFLITIGILAASYVNYLTSTLKNGWRYSLGGAAVPALILLIGSFFIHETPASLIERGKDEKGKQVLRKIRGIEDIELEFNEIKYATEVATKVKSPFKELFTKSENRPPLVCGTLLQFFQQFTGINVVMFYAPVLFQTMGSGDNASLISTVVTNGVNAIATVISLLVVDFAGRRCLLMEGALQMTATQMTIGGILLAHLKLVGPITGHAVR

SEQ ID NO: 27 - protein, 514 aa, Arabidopsis thaliana
MAGGFVSQTPGVRNYNYKLTPKVFVTCFIGAFGGLIFGYDLGISGGVTSMEPFLEEFFPYVYKKMKSAHENEYCRFDSQLLTLFTSSLYVAALVSSLFASTITRVFGRKWSMFLGGFTFFIGSAFNGFAQNIAMLLIGRILLGFGVGFANQSVPVYLSEMAPPNLRGAFNNGFQVAIIFGIVVATIINYFTAQMKGNIGWRISLGLACVPAVMIMIGALILPDTPNSLIERGYTEEAKEMLQSIRGTNEVDEEFQDLIDASEESKQVKHPWKNIMLPRYRPQLIMTCFIPFFQQLTGINVITFYAPVLFQTLGFGSKASLLSAMVTGIIELLCTFVSVFTVDRFGRRILFLQGGIQMLVSQIAIGAMIGVKFGVAGTGNIGKSDANLIVALICIYVAGFAWSWGPLGWLVPSEISPLEIRSAAQAINVSVNMFFTFLVAQLFLTMLCHMKFGLFFFFAFFVVIMTIFIYLMLPETKNVPIEEMNRVWKAHWFWGKFIPDEAVNMGAAEMQQKSV

SEQ ID NO: 28 - protein, 523 aa, Nicotiana tabacum; identical to SEQ ID NO: 22

SEQ ID NO: 29 - protein, 518 aa, Medicago truncatula
MAGGGIPIGGGNKEYPGNLTPFVTITCIVAAMGGLIFGYDIGISGGVTSMDPFLKKFFPAVYRKKNKDKSTNQYCQYDSQTLTMFTSSLYLAALLSSLVASTITRRFGRKLSMLFGGLLFLVGALINGFANHVWMLIVGRILLGFGIGFANQPVPLYLSEMAPYKYRGALNIGFQLSITIGILVANVLNYFFAKIKGGWGWRLSLGGAMVPALIITIGSLVLPDTPNSMIERGDRDGAKAQLKRIRGIEDVDEEFNDLVAASEASMQVENPWRNLLQRKYRPQLTMAVLIPFFQQFTGINVIMFYAPVLFNSIGFKDDASLMSAVITGVVNVVATCVSIYGVDKWGRRALFLEGGAQMLICQVAVAAAIGAKFGTSGNPGNLPEWYAIVVVLFICIYVAGFAWSWGPLGWLVPSEIFPLEIRSAAQSVNVSVNMLFTFLVAQVFLIMLCHMKFGLFLFFAFFVLVMSIYVFFLLPETKGIPIEEMDRVWKSHPFWSRFVEHGDHGNGVEMGKGAPKNV

SEQ ID NO: 30 - protein, 526 aa, Vitis vinifera
MEVGDGSFAPVGVSKQRADQYKGRLTTYVVVACLVAAVGGAIFGYDIGVSGGVTSMDTFLEKFFHTVYLKKRRAEEDHYCKYNDQGLAAFTSSLYLAGLVASIVASPITRKYGRRASIVCGGISFLIGAALNAAAVNLAMLLSGRIMLGIGIGFGDQAVPLYLSEMAPAHLRGALNMMFQLATTTGIFTANMINYGTAKLPSWGWRLSLGLAALPAILMTVGGLFLPETPNSLIERGSREKGRRVLERIRGTNEVDAEFEDIVDASELANSIKHPFRNILERRNRPQLVMAICMPAFQILNGINSILFYAPVLFQTMGFGNATLYSSALTGAVLVLSTVVSIGLVDRLGRRVLLISGGIQMVLCQVTVAIILGVKFGSNDGLSKGYSVLVVIVICLFVIAFGWSWGPLGWTVPSEIFPLETRSAGQSITVVVNLLFTFIIAQCFLSMLCSFKHGIFLFFAGWIVIMTLFVYFFLPETKGVPIEEMIFVWKKHWFWKRMVPGTPDVDDIDGLGSHSMESGGKTKLGS

SEQ ID NO: 31 - protein, 534 aa, Parachlorella kessleri
MAGGGVVVVSGRGLSTGDYRGGLTVYVVMVAFMAACGGLLLGYDNGVTGGVVSLEAFEKKFFPDVWAKKQEVHEDSPYCTYDNAKLQLFVSSLFLAGLVSCLFASWITRNWGRKVTMGIGGAFFVAGGLVNAFAQDMAMLIVGRVLLGFGVGLGSQVVPQYLSEVAPFSHRGMLNIGYQLFVTIGILIAGLVNYAVRDWENGWRLSLGPAAAPGAILFLGSLVLPESPNFLVEKGKTEKGREVLQKLCGTSEVDAEFADIVAAVEIARPITMRQSWASLFTRRYMPQLLTSFVIQFFQQFTGINAIIFYVPVLFSSLGSANSAALLNTVVVGAVNVGSTLIAVMFSDKFGRRFLLIEGGIQCCLAMLTTGVVLAIEFAKYGTDPLPKAVASGILAVICIFISGFAWSWGPMGWLIPSEIFTLETRPAGTAVAVVGNFLFSFVIGQAFVSMLCAMEYGVFLFFAGWLVIMVLCAIFLLPETKGVPIERVQALYARHWFWNRVMGPAAAEVIAEDEKRVAAASAIIKEEELSKAMK

SEQ ID NO: 32 - protein, 534 aa, Parachlorella kessleri; identical to SEQ ID NO: 13

SEQ ID NO: 33 - DNA, 2078 bp, Artificial sequence
  Synthetic construct
gccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaaccaccatggcgggcggcgccattgttgccagcggcggcgccagccgttcgagcgagtaccagggcggcctgaccgcctacgttctgctcgtggcgctggttgccgcctgcggcggcatgctgctgggctacgacaacggcgttaccggcggcgttgccagcatggagcagttcgagcgcaagttcttcccggacgtgtacgagaagaagcagcagattgtcgagaccagcccgtactgcacctacgacaacccgaagctccagctgttcgtgtcgagcctgttcctggcgggcctgattagctgcattttctcggcgtggattacccgcaactggggccgcaaggcgagcatgggcattggcggcattttcttcattgccgccggtggcctggttaacgccttcgcccaggacattgccatgctgattgtgggccgcgtcctgctgggcttcggcgttggcctgggcagccaggtggtgccacagtacctgagcgaggtggcgccattcagccatcgcggcatgctcaacattggctaccagctcttcgtgaccattggcattctgattgccggcctggtgaactacggcgtgcgcaactgggacaacggttggcgcctgagcctgggcctggcggcggttccaggcctgattctgctgctcggcgccatcgttctgccggagagcccgaacttcctggtggagaagggccgcaccgaccagggccgccgcattctggagaagctgcgcggcaccagccatgttgaggcggagttcgccgacattgtggcggcggtggagattgcccgcccaattaccatgcgccagagctggcgctcgctgttcacccgccgctacatgccacagctgctgaccagcttcgtgattcagttcttccagcagttcaccggcattaacgccatcattttctacgtgccggtgctgttcagcagcctgggctcggcgtcctcggcggcgctgctgaacaccgtggttgtgggcgccgtgaacgtgggcagcaccatgattgccgtgctgctgtcggacaagttcggccgccgcttcctgctgattgagggcggcattacctgctgcctggcgatgctggcggcgggcattacgctgggcgtggagttcggccagtacggcaccgaggacctgccacatccagtgtcggcgggcgtgctggcggtgatttgcattttcattgccggcttcgcctggagctggggcccaatgggctggctgattccgagcgagattttcaccctggagacccgcccagcgggcacggcggttgccgtgatgggcaacttcctgttctcgttcgtgattggccaggccttcgtgtcgatgctgtgcgcgatgaagttcggcgtgttcctgttcttcgccggctggctggtgattatggtgctgtgcgccattttcctgctgccggagaccaagggcgtgccgattgagcgcgtgcaggcgctgtacgcccgccactggttctggaagaaggtgatgggcccagcggcccaggagattattgccgaggacgagaagcgcgttgcggcgagccaggcgattatgaaggaggagcgcattagccagaccatgaagtaaccgacgtcgacccactctagaggatcgatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttg

SEQ ID NO: 34 - DNA, 208 bp, Chlamydomonas reinhardtii
gccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaa

(19) United States
(12) Patent Application Publication
(10) Pub. No.: US 2007/0166797 A1
(43) Pub. Date: Jul. 19, 2007

(54) Devices and solutions for prevention of sexually transmitted diseases

(75) Inventors: Harrison F. Dillon, Belmont, CA (US); Aravind Somanchi, Fremont, CA (US); Anwar Zaman, El Cerrito, CA (US)

Correspondence Address: SOLAZYME, INC., 3475 - T Edison Way, Menlo Park, CA, 94025, US

(73) Assignee: Solazyme, Inc., Menlo Park, CA (US)

(21) Appl. No.: 11/336,656

(22) Filed: Jan. 19, 2006

(51) Int. Cl. A61K 31/715; C08B 37/00; C12P 19/28; C12P 19/04

(52) U.S. Cl. 435/85; 435/101; 514/54; 536/53; 536/123

(57) ABSTRACT

Provided herein are antiviral compounds and methods of culturing microalgae to produce polysaccharides. Included in the invention are methods of producing novel polysaccharides with high antiviral avtivity. Also provided are methods of using polysaccharides for applications such as preventing or treating viral infections and providing prophylaxis and treatment for sexually transmitted diseases.

### BACKGROUND OF THE INVENTION

Carbohydrates have the general molecular formula CH₂O, and thus were once thought to represent “hydrated carbon”. However, the arrangement of atoms in carbohydrates has little to do with water molecules. Starch and cellulose are two common carbohydrates. Both are macromolecules with molecular weights in the hundreds of thousands. Both are polymers; that is, each is built from repeating units, monomers, much as a chain is built from its links.

Three common sugars share the same molecular formula: C₆H₁₂O₆. Because of their six carbon atoms, each is a hexose. Glucose is the immediate source of energy for cellular respiration. Galactose is a sugar in milk. Fructose is a sugar found in honey. Although all three share the same molecular formula (C₆H₁₂O₆), the arrangement of atoms differs in each case. Substances such as these three, which have identical molecular formulas but different structural formulas, are known as structural isomers. Glucose, galactose, and fructose are “single” sugars or monosaccharides.

Two monosaccharides can be linked together to form a “double” sugar or disaccharide. Three common disaccharides are sucrose, common table sugar (glucose+fructose); lactose, the major sugar in milk (glucose+galactose); and maltose, the product of starch digestion (glucose+glucose). Although the process of linking the two monomers is complex, the end result in each case is the loss of a hydrogen atom (H) from one of the monosaccharides and a hydroxyl group (OH) from the other. The resulting linkage between the sugars is called a glycosidic bond. The molecular formula of each of these disaccharides is C₁₂H₂₂O₁₁=2C₆H₁₂O₆—H2O. All sugars are very soluble in water because of their many hydroxyl groups. Although not as concentrated a fuel as fats, sugars are the most important source of energy for many cells.

### BRIEF SUMMARY OF THE INVENTION

The present invention relates to polysaccharides from microalgae. Representative polysaccharides include those present in the cell wall of microalgae as well as secreted polysaccharides, or exopolysaccharides. In addition to the polysaccharides themselves, such as in an isolated, purified, or semi-purified form, the invention includes a variety of compositions containing one or more microalgal polysaccharides as disclosed herein. The compositions include topical antiviral and pharmaceutical compositions which may be used for a variety of indications and uses as described herein. Other compositions include those containing one or more microalgal polysaccharides and a suitable carrier or excipient for topical or oral administration.

The invention further relates to methods of producing or preparing microalgal polysaccharides. In some disclosed methods, exogenous sugars are incorporated into the polysaccharides to produce polysaccharides distinct from those present in microalgae that do not incorporate exogenous sugars. The invention also includes methods of trophic conversion and recombinant gene expression in microalgae. In some methods, recombinant microalgae are prepared to express heterologous gene products, such as mammalian proteins as a non-limiting example, while in other embodiments, the microalgae are modified to produce more of a small molecule already made by microalgae in the absence of genetic modification.

Additionally, the invention relates to methods of using the polysaccharides and/or compositions containing them. In some disclosed methods, one or more polysaccharides are used to prevent or effect prophylaxis of sexually transmitted diseases and treat or effect prophylaxis of other infectious diseases.

In other aspects, the invention includes methods of preparing or producing a microalgal polysaccharide. In some aspects relating to an exopolysaccharide, the invention includes methods that separate the exopolysaccharide from other molecules present in the medium used to culture exopolysaccharide producing microalgae. In some embodiments, separation includes removal of the microalgae from the culture medium containing the exopolysaccharide, after the microalgae has been cultured for a period of time. Of course the methods may be practiced with microalgal polysaccharides other than exopolysaccharides. In other embodiments, the methods include those where the microalgae was cultured in a bioreactor, optionally where a gas is infused into the bioreactor.

In one embodiment, the invention includes a method of producing an exopolysaccharide, wherein the method comprises culturing microalgae in a bioreactor, wherein gas is infused into the bioreactor; separating the microalgae from culture media, wherein the culture media contains the exopolysaccharide; and separating the exopolysaccharide from other molecules present in the culture media.

The microalgae of the invention may be that of any species, including those listed in Table 1 herein. In some embodiments, the microalgae is a red algae, such as the red algae Porphyridium, which has two known species (Porphyridium sp. and Porphyridium cruentum) that have been observed to secrete large amounts of polysaccharide into their surrounding growth media. In other embodiments, the microalgae is of a genus selected from Rhodella, Chlorella, and Achnanthes. Non-limiting examples of species within a microalgal genus of the invention include Porphyridium sp., Porphyridium cruentum, Porphyridium purpureum, Porphyridium aerugineum, Rhodella maculata, Rhodella reticulata, Chlorella autotrophica, Chlorella stigmatophora, Chlorella capsulata, Achnanthes brevipes and Achnanthes longipes.

In some embodiments, a polysaccharide preparation method is practiced with culture media containing over 26.7, or over 27, mM sulfate (or total SO₄²⁻). Non-limiting examples include media with more than about 28, more than about 30, more than about 35, more than about 40, more than about 45, more than about 50, more than about 55, more than about 60, more than about 65, more than about 70, more than about 75, more than about 80, more than about 85, more than about 90, more than about 95, or more than about 100 mM sulfate. Sulfate in the media may be provided in one or more of the following forms: Na₂SO₄.10H₂O, MgSO₄.7H₂O, MnSO₄, and CuSO₄.

Other embodiments of the method include the separation of an exopolysaccharide from other molecules present in the culture media by tangential flow filtration. Alternatively, the methods may be practiced by separating an exopolysaccharide from other molecules present in the culture media by alcohol precipitation. Non-limiting examples of alcohols to use include ethanol, isopropanol, and methanol.

In other embodiments, a method may further comprise treating a polysaccharide or exopolysaccharide with a protease to degrade polypeptide (or proteinaceous) material attached to, or found with, the polysaccharide or exopolysaccharide. The methods may optionally comprise separating the polysaccharide or exopolysaccharide from proteins, peptides, and amino acids after protease treatment.

Other compositions of the invention may be formulated by subjecting a culture of microalgal cells and soluble exopolysaccharide to tangential flow filtration until the composition is substantially free of salts. Alternatively, a polysaccharide is prepared after proteolysis of polypeptides present with the polysaccharide. The polysaccharide and any contaminating polypeptides may be that of a culture medium separated from microalgal cells in a culture thereof. In some embodiments, the cells are of the genus Porphyridium.

In another embodiment, a method of preventing a sexually transmitted disease is described. In one embodiment, a method includes administration of a solution comprising a polysaccharide produced by microalgae and use of a prophylactic device. In other embodiments, the solution is administered via the device.

In a yet additional embodiment, a method of treating or effecting prophylaxis of viral infection is described. In one embodiment, a method includes administering a polysaccharide produced by microalgae to a mammal.

In further aspects, the invention describes recombinant methods to modify microalgal cells. In some embodiments, the methods produce a microalgal cell that expresses an exogenous gene product. The exogenous gene product may encode a carbohydrate transporter protein as a non-limiting example. The recombinantly modified cells per se, whether newly created or maintained in culture, are also part of the invention.

The invention also describes methods of recombinantly modifying a microalgal cell. In some embodiments, a method of trophically converting a microalgal cell, such as members of the genus Porphyridium, is described. The method may include selecting cells for a phenotype after transforming cells with a nucleic acid molecule in an expressible form. In some methods, the phenotype may be the ability to undergo cell division in the absence of light and/or in the presence of a carbohydrate that is transported by a carbohydrate transporter protein encoded by the nucleic acid molecule.

These methods may also be considered a method of expressing an exogenous gene in a microalgal cell. The method may include use of an expression vector containing a nucleic acid sequence encoding a polypeptide, such as a carbohydrate transporter protein. Alternatively, the method may include transforming a microalgal cell with a dual expression vector containing 1) a resistance cassette with a gene encoding a protein that confers resistance to an antibiotic, such as zeocin as a non-limiting example, operably linked to a promoter active in microalgae; and 2) a second expression cassette with a gene encoding a second protein operably linked to a promoter active in microalgae. After transformation, cells may be selected for the ability to survive in the presence of the antibiotic, such as at least 2.5 μg/ml zeocin as a non-limiting example where zeocin resistance is used. Alternatively, the antibiotic can be at least 3.0 μg/ml zeocin, at least 4.0 μg/ml zeocin, at least 5.0 μg/ml zeocin, at least 6.0 μg/ml zeocin, at least 7.0 μg/ml zeocin, and at least 8.0 μg/ml zeocin.

The invention further relates to microalgal cells expressing a carbohydrate transporter protein for use in a method of producing a glycopolymer. In some embodiments, the method may include providing a transgenic cell containing an expressible gene encoding a monosaccharide transporter; and culturing the cell in the presence of at least one monosaccharide, transported into the cell by the transporter, wherein the monosaccharide is incorporated into a polysaccharide made by the cell.

Alternatively, a method of trophically converting a microalgae cell may include selecting for the ability to undergo cell division in the absence of light after subjecting the microalgal cell to a mutagen and placing the cell in the presence of a molecule listed in Tables 2 or 3 herein.

The details of additional embodiments of the invention are set forth in the accompanying drawings and the description below. Other features and advantages of the invention will be apparent from the drawings and detailed description, and from the claims.

### BRIEF DESCRIPTION OF THE DRAWINGS

FIG. 1 shows precipitation of 4 liters of Porphyridium cruentum exopolysaccharide using 38.5% isopropanol. (a) supernatant; (b) addition of 38.5% isopropanol; (c) precipitated polysaccharide; (d) separating step.

FIG. 2 shows Porphyridium sp. cultured on agar plates containing various concentrations of zeocin.

FIG. 3 shows growth of Porphyridium sp. and Porphyridium cruentum cells grown in light in the presence of various concentrations of glycerol.

FIG. 4 shows Porphyridium sp. cells grown in the dark in the presence of various concentrations of glycerol.

FIG. 5 shows sexually transmitted disease prevention devices containing various amounts of exopolysaccharide.

FIG. 6 shows protein concentration measurements of autoclaved, protease-treated, and diafiltered exopolysaccharide.

### DETAILED DESCRIPTION OF THE INVENTION

U.S. patent application Ser. No. 10/411,910 is hereby incorporated in its entirety for all purposes. U.S. patent application Ser. No. ______, filed Jan. 19, 2006, entitled “Polysaccharide Compositions and Methods of Administering, Producing, and Formulating Polysaccharide Compositions”, is hereby incorporated in its entirety for all purposes. All other references cited are incorporated in their entirety for all purposes.

Definitions: The following definitions are intended to convey the intended meaning of terms used throughout the specification and claims, however they are not limiting in the sense that minor or trivial differences fall within their scope.

“Active in microalgae” means a nucleic acid that is functional in microalgae. For example, a promoter that has been used to drive an antibiotic resistance gene to impart antibiotic resistance to a transgenic microalgae is active in microalgae. Nonlimiting examples of promoters active in microalgae are promoters endogenous to certain algae species and promoters found in plant viruses.

“Antibody” means human antibodies, non-human antibodies, humanized antibodies, single chain or other antibody fragments such as scFv, Fc, and other fragments, and other antibody derivatives that specifically bind an antigen.

“Antiviral lubricant” means a molecule that possesses both antiviral activity and lubricant activity.

“Axenic” means a culture of an organism that is free from contamination by other living organisms.

“Bioreactor” means an enclosure or partial enclosure in which cells are cultured in suspension.

“Carbohydrate modifying enzyme” means an enzyme that utilizes a carbohydrate as a substrate and structurally modifies the carbohydrate.

“Carbohydrate transporter” means a polypeptide that resides in a lipid bilayer and facilitates the transport of carbohydrates across the lipid bilayer.

“Carrier suitable for topical administration” means a compound that may be administered, together with one or more compounds of the present invention, and which does not destroy the activity thereof and is nontoxic when administered in concentrations and amounts sufficient to deliver the compound to the skin or a mucosal tissue.

“Conditions favorable to cell division” means conditions in which cells divide at least once every 72 hours.

“Endopolysaccharide” means a polysaccharide that is retained intracellularly.

“Exogenous gene” means a gene transformed into a wild-type organism. The gene can be heterologous from a different species, or homologous from the same species, in which case the gene occupies a different location in the genome of the organism than the endogenous gene.

“Exogenously provided” describes a molecule provided to the culture media of a cell culture.

“Exopolysaccharide” means a polysaccharide that is secreted from a cell into the extracellular environment.

“Filtrate” means the portion of a tangential flow filtration sample that has passed through the filter.

“Fixed carbon source” means molecule(s) containing carbon that are present at ambient temperature and pressure in solid or liquid form.

“Glycopolymer” means a biologically produced molecule comprising at least two monosaccharides. Examples of glycopolymers include glycosylated proteins, polysaccharides, oligosaccharides, and disaccharides.

“Microalgae” means a single-celled organism that is capable of performing photosynthesis. Microalgae include obligate photoautotrophs, which cannot metabolize a fixed carbon source as energy, as well as heterotrophs, which can live solely off of light, solely off of a fixed carbon source, or a combination of the two.

“Naturally produced” describes a compound that is produced by a wild-type organism.

“Pharmaceutically acceptable carrier or adjuvant” refers to a carrier or adjuvant that may be administered to a patient, together with one or more compounds of the present invention, and which does not destroy the pharmacological activity thereof and is nontoxic when administered in doses sufficient to deliver a therapeutic amount of the compound.

“Photobioreactor” means a waterproof container, at least part of which is at least partially transparent, allowing light to pass through, in which one or more microalgae cells are cultured. Photobioreactors may be sealed, as in the instance of a polyethylene bag, or may be open to the environment, as in the instance of a pond.

“Polysaccharide material” is a composition that contains more than one species of polysaccharide, and optionally contaminants such as proteins, lipids, and nucleic acids, such as, for example, a microalgal cell homogenate.

“Polysaccharide” means a compound or preparation containing one or more molecules that contain at least two saccharide molecules covalently linked. A “polysaccharide”, “endopolysaccharide” or “exopolysaccharide” can be a preparation of polymer molecules that have similar or identical repeating units but different molecular weights within the population.

“Port”, in the context of a photobioreactor, means an opening in the photobioreactor that allows influx or efflux of materials such as gases, liquids, and cells. Ports are usually connected to tubing leading to and/or from the photobioreactor.

“Red microalgae” means unicellular algae that is of the list of classes comprising Bangiophyceae, Florideophyceae, Goniotrichales, or is otherwise a member of the Rhodophyta.

“Retentate” means the portion of a tangential flow filtration sample that has not passed through the filter.

“Selectively binds to” refers to a binding reaction which is determinative of the presence of a molecule in the presence of a heterogeneous population of other molecules. Thus, under designated conditions, a specified ligand binds preferentially to a particular molecule and does not bind in a significant amount to other proteins present in the sample. A molecule such as antibody that specifically binds to a protein often has an association constant of at least 10⁶ M⁻¹, or 10⁷ M⁻¹, preferably 10⁸ M⁻¹ to 10⁹ M⁻¹, and more preferably, about 10¹⁰ M⁻¹ to 10¹¹ M⁻¹ or higher. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

“Substantially free of protein” means compositions that are preferably of high purity and are substantially free of potentially harmful contaminants, including proteins (e.g., at least National Food (NF) grade, generally at least analytical grade, and more typically at least pharmaceutical grade). Compositions are at least 80, at least 90, at least 99 or at least 99.9% w/w pure of undesired contaminants such as proteins are substantially free of protein. To the extent that a given compound must be synthesized prior to use, the resulting product is typically substantially free of any potentially toxic agents, particularly any endotoxins, which may be present during the synthesis or purification process. Compositions are usually made under GMP conditions. Compositions for parenteral administration are usually sterile and substantially isotonic.

I General

Polysaccharides form a heterogeneous group of polymers of different length and composition. They are constructed from monosaccharide residues that are linked by glycosidic bonds. Glycosidic linkages may be located between the C₁ (or C₂) of one sugar residue and the C₂, C₃, C₄, C₅ or C₆ of the second residue. A branched sugar results if more than two types of linkage are present in single monosaccharide molecule.

Monosaccharides are simple sugars with multiple hydroxyl groups. Based on the number of carbons (e.g., 3, 4, 5, or 6) a monosaccharide is a triose, tetrose, pentose, or hexose. Pentoses and hexoses can cyclize, as the aldehyde or keto group reacts with a hydroxyl on one of the distal carbons. Examples of monosaccharides are galactose, glucose, and rhamnose.

Polysaccharides are molecules comprising a plurality of monosaccharides covalently linked to each other through glycosidic bonds. Polysaccharides consisting of a relatively small number of monosaccharide units, such as 10 or less, are sometimes referred to as oligosaccharides. The end of the polysaccharide with an anomeric carbon (C₁) that is not involved in a glycosidic bond is called the reducing end. A polysaccharide may consist of one monosaccharide type, known as a homopolymer, or two or more types of monosaccharides, known as a heteropolymer. Examples of homopolysaccharides are cellulose, amylose, inulin, chitin, chitosan, amylopectin, glycogen, and pectin. Amylose is a glucose polymer with α(1→4) glycosidic linkages. Amylopectin is a glucose polymer with α(1→4) linkages and branches formed by α(1→6) linkages. Examples of heteropolysaccharides are glucomannan, galactoglucomannan, xyloglucan, 4-O-methylglucuronoxylan, arabinoxylan, and 4-O-Methylglucuronoarabinoxylan.

Polysaccharides can be structurally modified both enzymatically and chemically. Examples of modifications include sulfation, phosphorylation, methylation, O-acetylation, fatty acylation, amino N-acetylation, N-sulfation, branching, and carboxyl lactonization.

Glycosaminoglycans are polysaccharides of repeating disaccharides. Within the disaccharides, the sugars tend to be modified, with acidic groups, amino groups, sulfated hydroxyl and amino groups. Glycosaminoglycans tend to be negatively charged, because of the prevalence of acidic groups. Examples of glycosaminoglycans are heparin, chondroitin, and hyaluronic acid.

Polysaccharides are produced in eukaryotes mainly in the endoplasmic reticulum (ER) and Golgi apparatus. Polysaccharide biosynthesis enzymes are usually retained in the ER, and amino acid motifs imparting ER retention have been identified (Gene. 2000 Dec. 31; 261(2):321-7). Polysaccharides are also produced by some prokaryotes, such as lactic acid bacteria.

Polysaccharides that are secreted from cells are known as exopolysaccharides. Many types of cell walls, in plants, algae, and bacteria, are composed of polysaccharides. The cell walls are formed through secretion of polysaccharides. Some species, including algae and bacteria, secrete polysaccharides that are released from the cells. In other words, these molecules are not held in association with the cells as are cell wall polysaccharides. Instead, these molecules are released from the cells. For example, cultures of some species of microalgae secrete exopolysaccharides that are suspended in the culture media.

II Methods of Producing Polysaccharides

A. Cell Culture Methods: Microalgae

Polysaccharides can be produced by culturing microalgae. Examples of microalgae that can be cultured to produce polysaccharides are shown in Table 1. Also listed are references that enable the skilled artisan to culture the microalgae species under conditions sufficient for polysaccharide production. Also listed are strain numbers from various publicly available algae collections, as well as strains published in journals that require public dissemination of reagents as a prerequisite for publication.

TABLE 1

| | | Culture and | | |
|---|---|---|---|---|
| | | polysaccharide | | |
| | | purification method | Monosaccharide | |
| Species | Strain Number/Source | reference | Composition | Culture conditions |
| Porphyridium | UTEX¹ 161 | M. A. Guzman-Murillo | Xylose, | Cultures obtained from various sources and were |
| cruentum | | and F. Ascencio., Letters | Glucose, | cultured in F/2 broth prepared with seawater |
| | | in Applied Microbiology | Galactose, | filtered through a 0.45 um Millipore filter or |
| | | 2000, 30, 473-478 | Glucoronic | distilled water depending on microalgae salt |
| | | | acid | tolerance. Incubated at 25° C. in flasks and |
| | | | | illuminated with white fluorescent lamps. |
| Porphyridium | UTEX 161 | Fabregas et al., Antiviral | Xylose, | Cultured in 80 ml glass tubes with aeration of |
| cruentum | | Research 44(1999)-67-43 | Glucose, | 100 ml/min and 10% CO₂, for 10 s every ten minutes |
| | | | Galactose and | to maintain pH > 7.6. Maintained at 22° in 12:12 |
| | | | Glucoronic | Light/dark periodicity. Light at 152.3 umol/m2/s. |
| | | | acid | Salinity 3.5% (nutrient enriched as Fabregas, 1984 |
| | | | | modified in 4 mmol Nitrogen/L) |
| Porphyridium sp. | UTEX 637 | Dvir, Brit. J. of Nutrition | Xylose, | Outdoor cultivation for 21 days in artficial sea |
| | | (2000), 84, 469-476. | Glucose and | water in polyethylene sleeves. See Jones (1963) |
| | | [Review: S. Geresh | Galactose, | and Cohen & Malis Arad, 1989) |
| | | Biosource Technology 38 | Methyl | |
| | | (1991) 195-201]- | hexoses, | |
| | | Huleihel, 2003, Applied | Mannose, | |
| | | Spectoscopy, v57, No. 4 | Rhamnose | |
| | | 2003 | | |
| Porphyridium | SAG² 111.79 | Talyshinsky, Marina | xylose, | see Dubinsky et al. Plant Physio. And Biochem. |
| aerugineum | | Cancer Cell Int'l 2002, 2; | glucose, | (192) 30: 409-414. Pursuant to Ramus_1972--> |
| | | Review: S. Geresh | galactose, | Axenic culutres are grown in MCYII liquid |
| | | Biosource Technology 38 | methyl | medium at 25° C. and illuminated with Cool White |
| | | (1991) 195-201]1 See | hexoses | fluorescent tubes on a 16:8 hr light dark cycle. |
| | | Ramus_1972 | | Cells kept in suspension by agitation on a |
| | | | | gyrorotary shaker or by a stream of filtered air. |
| Porphyridium | strain 1380-1a | Schmitt D., Water | unknown | See cited reference |
| purpurpeum | | Research | | |
| | | Volume 35, Issue 3, | | |
| | | March 2001, Pages 779-785, | | |
| | | Bioprocess Biosyst | | |
| | | Eng. 2002 Apr; 25(1): 35-42. | | |
| | | Epub 2002 Mar 6 | | |
| Chaetoceros sp. | USCE³ | M. A. Guzman-Murillo | unknown | See cited reference |
| | | and F. Ascencio., Letters | | |
| | | in Applied Microbiology | | |
| | | 2000, 30, 473-478 | | |
| Chlorella | USCE | M. A. Guzman-Murillo | unknown | See cited reference |
| autotropica | | and F. Ascencio., Letters | | |
| | | in Applied Microbiology | | |
| | | 2000, 30, 473-478 | | |
| Chlorella | UTEX 580 | Fabregas et al., Antiviral | unknown | Cultured in 80 ml glass tubes with aeration of |
| autotropica | | Research 44(1999)-67-43 | | 100 ml/min and 10% CO2, for 10 s every ten |
| | | | | minutes to maintain pH > 7.6. Maintained at 22° in |
| | | | | 12:12 Light/dark periodicity. Light at 152.3 |
| | | | | umol/m2/s. Salinity 3.5% (nutrient enriched as |
| | | | | Fabregas, 1984) |
| Chlorella | UTEX LB2074 | M. A. Guzman-Murillo | Unknown | Cultures obtained from various sources and were |
| capsulata | | and F. Ascencio., Letters | | cultured in F/2 broth prepared with seawater |
| | | in Applied Microbiology | | filtered through a 0.45 um Millipore filter or |
| | | 2000, 30, 473-478 | | distilled water depending on microalgae salt |
| | | | | tolerance. Incubated at 25° C. in flasks and |
| | | | | illuminated with white fluorescent lamps. |
| Chlorella | GGMCC⁴ | S. Guzman, Phytotherapy | glucose, | Grown in 10 L of membrane filtered (0.24 um) |
| stigmatophora | | Rscrh (2003) 17: 665-670 | glucuronic | seawater and sterilized at 120° for 30 min and |
| | | | acid, xylose, | enriched with Erd Schreiber medium. Cultures |
| | | | ribose/fucose | maintained at 18 +/− 1° C. under constant 1% CO₂ |
| | | | | bubbling. |
| Dunalliela | DCCBC⁵ | Fabregas et al., Antiviral | unknown | Cultured in 80 ml glass tubes with aeration of |
| tertiolecta | | Research 44(1999)-67-43 | | 100 ml/min and 10% CO2, for 10 s every ten |
| | | | | minutes to maintain pH > 7.6. Maintained at 22° in |
| | | | | 12:12 Light/dark periodicity. Light at 152.3 |
| | | | | umol/m2/s. Salinity 3.5% (nutrient enriched as |
| | | | | Fabregas, 1984) |
| Dunalliela | DCCBC | Fabregas et al., Antiviral | unknown | Cultured in 80 ml glass tubes with aeration of |
| bardawil | | Research 44(1999)-67-43 | | 100 ml/min and 10% CO2, for 10 s every ten |
| | | | | minutes to maintain pH > 7.6. Maintained at 22° in |
| | | | | 12:12 Light/dark periodicity. Light at 152.3 |
| | | | | umol/m²/s. Salinity 3.5% (nutrient enriched as |
| | | | | Fabregas, 1984) |
| Isochrysis | HCTMS⁶ | M. A. Guzman-Murillo | unknown | Cultures obtained from various sources and were |
| galbana var. | | and F. Ascencio., Letters | | cultured in F/2 broth prepared with seawater |
| tahitiana | | in Applied Microbiology | | filtered through a 0.45 um millipore filter or |
| | | 2000, 30, 473-478 | | distilled water depending on microalgae salt |
| | | | | tolerance. Incubated at 25° C. in flasks and |
| | | | | illuminated with white fluorescent lamps. |
| Isochrysis | UTEX LB 987 | Fabregas et al., Antiviral | unknown | Cultured in 80 ml glass tubes with aeration of |
| galbana var. | | Research 44(1999)-67-43 | | 100 ml/min and 10% CO2, for 10 s every ten |
| Tiso | | | | minutes to maintain pH > 7.6. Maintained at 22° in |
| | | | | 12:12 Light/dark periodicity. Light at 152.3 |
| | | | | umol/m²/s. Salinity 3.5% (nutrient enriched as |
| | | | | Fabregas, 1984) |
| Isochrysis sp. | CCMP⁷ | M. A. Guzman-Murillo | unknown | Cultures obtained from various sources and were |
| | | and F. Ascencio., Letters | | cultured in F/2 broth prepared with seawater |
| | | in Applied Microbiology | | filtered through a 0.45 um Millipore filter or |
| | | 2000, 30, 473-478 | | distilled water depending on microalgae salt |
| | | | | tolerance. Incubated at 25° C. in flasks and |
| | | | | illuminated with white fluorescent lamps. |
| Phaeodactylum | UTEX 642, 646, | M. A M. A. Guzman- | unknown | Cultures obtained from various sources and were |
| tricornutum | 2089 | Murillo and F. Ascencio., | | cultured in F/2 broth prepared with seawater |
| | | Letters in Applied | | filtered through a 0.45 um Millipore filter or |
| | | Microbiology 2000, 30, | | distilled water depending on microalgae salt |
| | | 473-478 | | tolerance. Incubated at 25° C. in flasks and |
| | | | | illuminated with white fluorescent lamps. |
| Phaeodactylum | GGMCC | S. Guzman, Phytotherapy | glucose, | Grown in 10 L of membrane filtered (0.24 um) |
| tricornutum | | Rscrh (2003) 17: 665-670 | glucuronic | seawater and sterilized at 120° for 30 min and |
| | | | acid, and | enriched with Erd Schreiber medium. Cultures |
| | | | mannose | maintained at 18 +/− 1° C. under constant 1% CO2 |
| | | | | bubbling. |
| Tetraselmis sp. | CCMP 1634-1640; | M. A. Guzman-Murillo | unknown | Cultures obtained from various sources and were |
| | UTEX 2767 | and F. Ascencio., Letters | | cultured in F/2 broth prepared with seawater |
| | | in Applied Microbiology | | filtered through a 0.45 um Millipore filter or |
| | | 2000, 30, 473-478 | | distilled water depending on microalgae salt |
| | | | | tolerance. Incubated at 25° C. in flasks and |
| | | | | illuminated with white fluorescent lamps. |
| Botrycoccus | UTEX 572 and | M. A. Guzman-Murillo | unknown | Cultures obtained from various sources and were |
| braunii | 2441 | and F. Ascencio., Letters | | cultured in F/2 broth prepared with seawater |
| | | in Applied Microbiology | | filtered through a 0.45 um Millipore filter or |
| | | 2000, 30, 473-478 | | distilled water depending on microalgae salt |
| | | | | tolerance. Incubated at 25° C. in flasks and |
| | | | | illuminated with white fluorescent lamps. |
| Cholorococcum | UTEX 105 | M. A. Guzman-Murillo | unknown | Cultures obtained from various sources and were |
| | | and F. Ascencio., Letters | | cultured in F/2 broth prepared with seawater |
| | | in Applied Microbiology | | filtered through a 0.45 um Millipore filter or |
| | | 2000, 30, 473-478 | | distilled water depending on microalgae salt |
| | | | | tolerance. Incubated at 25° C. in flasks and |
| | | | | illuminated with white fluorescent lamps. |
| Hormotilopsis | UTEX 104 | M. A. Guzman-Murillo | unknown | Cultures obtained from various sources and were |
| gelatinosa | | and F. Ascencio., Letters | | cultured in F/2 broth prepared with seawater |
| | | in Applied Microbiology | | filtered through a 0.45 um Millipore filter or |
| | | 2000, 30, 473-478 | | distilled water depending on microalgae salt |
| | | | | tolerance. Incubated at 25° C. in flasks and |
| | | | | illuminated with white fluorescent lamps. |
| Neochloris | UTEX 1185 | M. A. Guzman-Murillo | unknown | Cultures obtained from various sources and were |
| oleoabundans | | and F. Ascencio., Letters | | cultured in F/2 broth prepared with seawater |
| | | in Applied Microbiology | | filtered through a 0.45 um Millipore filter or |
| | | 2000, 30, 473-478 | | distilled water depending on microalgae salt |
| | | | | tolerance. Incubated at 25° C. in flasks and |
| | | | | illuminated with white fluorescent lamps. |
| Ochromonas | UTEX L1298 | M. A. Guzman-Murillo | unknown | Cultures obtained from various sources and were |
| Danica | | and F. Ascencio., Letters | | cultured in F/2 broth prepared with seawater |
| | | in Applied Microbiology | | filtered through a 0.45 um Millipore filter or |
| | | 2000, 30, 473-478 | | distilled water depending on microalgae salt |
| | | | | tolerance. Incubated at 25° C. in flasks and |
| | | | | illuminated with white fluorescent lamps. |
| Gyrodinium | KG03; KGO9; | Yim, Joung Han et. Al., J. | Homopolysac | Isolated from seawater collected from red-tide |
| impudicum | KGJO1 | of Microbiol December 2004, | charide of | bloom in Korean coastal water. Maintained in f/2 |
| | | 305-14; Yim, J. H. (2000) | galactose w/ | medium at 22° under circadian light at |
| | | Ph.D. Dissertations, | 2.96% uronic | 100 uE/m2/sec: dark cycle of 14 h: 10 h for 19 days. |
| | | University of Kyung Hee, | acid | Selected with neomycin and/or cephalosporin |
| | | Seoul | | 20 ug/ml |
| Ellipsoidon sp. | See cited | Fabregas et al., Antiviral | unknown | Cultured in 80 ml glass tubes with aeration of |
| | references | Research 44(1999)-67-43; | | 100 ml/min and 10% CO2, for 10 s every ten |
| | | Lewin, R. A. Cheng, | | minutes to maintain pH > 7.6. Maintained at 22° in |
| | | L., 1989. Phycologya 28, | | 12:12 Light/dark periodicity. Light at 152.3 |
| | | 96-108 | | umol/m2/s. Salinity 3.5% (nutrient enriched as |
| | | | | Fabregas, 1984) |
| Rhodella | UTEX 2320 | Talyshinsky, Marina | unknown | See Dubinsky O. et al. Composition of Cell wall |
| reticulata | | Cancer Cell Int'l 2002, 2 | | polysaccharide produced by unicellular red algae |
| | | | | Rhodella reticulata. 1992 Plant Physiology and |
| | | | | biochemistry 30: 409-414 |
| Rhodella | UTEX LB 2506 | Evans, LV., et al. J. Cell | Galactose, | Grown in either SWM3 medium or ASP12, MgCl2 |
| maculata | | Sci 16, 1-21(1974); | xylose, | supplement. 100 mls in 250 mls volumetric |
| | | EVANS, L. V. (1970). | glucuronic | Erlenmeyer flask with gentle shaking and 40001x |
| | | Br. phycol. J. 5, 1-13. | acid | Northern Light fluorescent light for 16 hours. |
| Gymnodinium sp. | Oku-1 | Sogawa, K., et al., Life | unknown | See cited reference |
| | | Sciences, Vol. 66, No. 16, | | |
| | | pp. PL 227-231 (2000) | | |
| | | AND Umermura, Ken: | | |
| | | Biochemical | | |
| | | Pharmacology 66 (2003) | | |
| | | 481-487 | | |
| Spirilina | UTEX LB 1926 | Kaji, T et. Al., Life Sci | Na-Sp | See cited reference |
| platensis | | 2002 Mar 8; 70(16): 1841-8 | contains two | |
| | | Schaeffer and Krylov | disaccharide | |
| | | (2000) Review- | repeats: | |
| | | Ectoxicology and | Aldobiuronic | |
| | | Environmental Safety. | acid and | |
| | | 45, 208-227. | Acofriose + | |
| | | | other minor | |
| | | | saccharides | |
| | | | and sodium | |
| | | | ion | |
| Cochlodinuium | Oku-2 | Hasui., et. Al., Int. J. Bio. | mannose, | Precultures grown in 500 ml conicals containing |
| polykrikoides | | Macromol. Volume 17 | galactose, | 300 mls ESM (?) at 21.5° C. for 14 days in |
| | | No. 5 1995. | glucose and | continuous light (3500 lux) in growth cabinet) and |
| | | | uronic acid | then transferred to 5 liter conical flask containing 3 |
| | | | | liters of ESM. Grown 50 days and then filtered thru |
| | | | | wortmann GFF filter. |
| Nostoc | PCC⁸ 7413, | Sangar, VK Applied | unknown | Growth in nitrogen fixing conditions in BG-11 |
| muscorum | 7936, 8113 | Micro. (1972) & A. M. | | medium in aerated cultures maintained in log phase |
| | | Burja et al Tetrahydron | | for several months. 250 mL culture media that were |
| | | 57 (2001) 937-9377; | | disposed in a temperature controlled incubator and |
| | | Otero A., J Biotechnol. | | continuously illuminated with 70 umol photon m-2 |
| | | 2003 Apr 24; 102(2): 143-52 | | s-1 at 30° C. |
| Cyanospira | See cited | A. M. Burja et al. | unknown | See cited reference |
| capsulata | references | Tetrahydron 57 (2001) | | |
| | | 937-9377 & Garozzo, D., | | |
| | | Carbohydrate Res. 1998 | | |
| | | 307 113-124; Ascensio, | | |
| | | F., Folia Microbiol | | |
| | | (Praha). 2004; 49(1): 64-70., | | |
| | | Cesaro, A., et al., Int | | |
| | | J Biol Macromol. 1990 | | |
| | | Apr; 12(2): 79-84 | | |
| Cyanothece sp. | ATCC 51142 | Ascensio F., Folia | unknown | Maintained at 27° C. in ASN III medium with |
| | | Microbiol (Praha). | | light/dark cycle of 16/8 h under fluorescent light of |
| | | 2004; 49(1): 64-70. | | 3,000 lux light intensity. In Phillips each of 15 |
| | | | | strains were grown photoautotrophically in |
| | | | | enriched seawater medium. When required the |
| | | | | amount of NaNO3 was reduced from 1.5 to 0.35 |
| | | | | g/L. Strains axenically grown in an atmosphere of |
| | | | | 95% air and 5% CO2 for 8 days under continuous |
| | | | | illumination. with mean photon flux of 30 umol |
| | | | | photon/m2/s for the first 3 days of growth and 80 |
| | | | | umol photon/m/s |
| Chlorella | UTEX 343; | Cheng_2004 Journal of | unknown | See cited reference |
| pyrenoidosa | UTEX 1806 | Medicinal Food 7(2) | | |
| | | 146-152 | | |
| Phaeodactylum | CCAP 1052/1A | Fabregas et al., Antiviral | unknown | Cultured in 80 ml glass tubes with aeration of |
| tricornutum | | Research 44(1999)-67-43 | | 100 ml/min and 10% CO2, for 10 s every ten |
| | | | | minutes to maintain pH > 7.6. Maintained at 22° in |
| | | | | 12:12 Light/dark periodicity. Light at 152.3 |
| | | | | umol/m2/s. Salinity 3.5% (nutrient enriched as |
| | | | | Fabregas, 1984) |
| Chlorella | USCE | M. A. Guzman-Murillo | unknown | See cited reference |
| autotropica | | and F. Ascencio., Letters | | |
| | | in Applied Microbiology | | |
| | | 2000, 30, 473-478 | | |
| Chlorella sp. | CCM | M. A. Guzman-Murillo | unknown | See cited reference |
| | | and F. Ascencio., Letters | | |
| | | in Applied Microbiology | | |
| | | 2000, 30, 473-478 | | |
| Dunalliela | USCE | M. A. Guzman-Murillo | unknown | See cited reference |
| tertiolecta | | and F. Ascencio., Letters | | |
| | | in Applied Microbiology | | |
| | | 2000, 30, 473-478 | | |
| Isochrysis | UTEX LB 987 | Fabregas et al., Antiviral | unknown | Cultured in 80 ml glass tubes with aeration of |
| galabana | | Research 44(1999)-67-43 | | 100 ml/min and 10% CO₂, for 10 s every ten minutes |
| | | | | to maintain pH > 7.6. Maintained at 22° in 12:12 |
| | | | | Light/dark periodicity. Light at 152.3 umol/m2/s. |
| | | | | Salinity 3.5% (nutrient enriched as Fabregas, 1984) |
| Tetraselmis | CCAP 66/1 A-D | Fabregas et al., Antiviral | unknown | Cultured in 80 ml glass tubes with aeration of |
| tetrathele | | Research 44(1999)-67-43 | | 100 ml/min and 10% CO₂, for 10 s every ten minutes |
| | | | | to maintain pH > 7.6. Maintained at 22° in 12:12 |
| | | | | Light/dark periodicity. Light at 152.3 umol/m2/s. |
| | | | | Salinity 3.5% (nutrient enriched as Fabregas, 1984) |
| Tetraselmis | UTEX LB 2286 | M. A. Guzman-Murillo | unknown | See cited reference |
| suecica | | and F. Ascencio., Letters | | |
| | | in Applied Microbiology | | |
| | | 2000, 30, 473-478 | | |
| Tetraselmis | CCAP 66/4 | Fabregas et al., Antiviral | unknown | Cultured in 80 ml glass tubes with aeration of |
| suecica | | Research 44(1999)-67-43 | | 100 ml/min and 10% CO₂, for 10 s every ten minutes |
| | | and Otero and Fabregas- | | to maintain pH > 7.6. Maintained at 22° in 12:12 |
| | | Aquaculture 159 (1997) | | Light/dark periodicity. Light at 152.3 umol/m2/s. |
| | | 111-123 | | Salinity 3.5% (nutrient enriched as Fabregas, 1984) |
| Botrycoccus | UTEX 2629 | M. A. Guzman-Murillo | unknown | See cited reference |
| sudeticus | | and F. Ascencio., Letters | | |
| | | in Applied Microbiology | | |
| | | 2000, 30, 473-478 | | |
| Chlamydomon | UTEX 729 | Moore and Tisher | unknown | See cited reference |
| as mexicana | | Science. 1964 Aug | | |
| | | 7; 145: 586-7. | | |
| Dysmorphococcus | UTEX LB 65 | M. A. Guzman-Murillo | unknown | See cited reference |
| globosus | | and F. Ascencio., Letters | | |
| | | in Applied Microbiology | | |
| | | 2000, 30, 473-478 | | |
| Rhodella | UTEX LB 2320 | S. Geresh et al., J | unknown | See cited reference |
| reticulata | | Biochem. Biophys. | | |
| | | Methods 50 (2002) 179-187 | | |
| | | [Review: S. Geresh | | |
| | | Biosource Technology 38 | | |
| | | (1991) 195-201] | | |
| Anabena | ATCC 29414 | Sangar, VK Appl | In Vegative | See cited reference |
| cylindrica | | Microbiol. 1972 | wall where | |
| | | Nov; 24(5): 732-4 | only 18% is | |
| | | | carbohydrate- | |
| | | | Glucose | |
| | | | [35%], | |
| | | | mannose | |
| | | | [50%], | |
| | | | galactose, | |
| | | | xylose, and | |
| | | | fucose. In | |
| | | | heterocyst | |
| | | | wall where | |
| | | | 73% is | |
| | | | carbohydrate- | |
| | | | Glucose 73% | |
| | | | and Mannose | |
| | | | is 21% with | |
| | | | some | |
| | | | galactose and | |
| | | | xylose | |
| Anabena | A37; JM | Moore, BG [1965] Can J. | Glucose and | See cited reference and APPLIED |
| flosaquae | Kingsbury | Microbiol. | mannose | ENVIRONMENTAL MICROBIOLOGY, April |
| | Laboratory, | Dec; 11(6): 877-85 | | 1978, 718-723) |
| | Cornell | | | |
| | University | | | |
| Palmella | See cited | Sangar, VK Appl | unknown | See cited reference |
| mucosa | references | Microbiol. 1972 | | |
| | | Nov; 24(5): 732-4; Lewin | | |
| | | RA., (1956) Can J | | |
| | | Microbiol. 2: 665-672; | | |
| | | Arch Mikrobiol. 1964 | | |
| | | Aug 17; 49: 158-66 | | |
| Anacystis | PCC 6301 | Sangar, VK Appl | Glucose, | See cited reference |
| nidulans | | Microbiol. 1972 | galactose, | |
| | | Nov; 24(5): 732-4 | mannose | |
| Phormidium | See cited | Vicente-Garcia V. et al., | Galactose, | Cultivated in 2 L BG-11 medium at 28° C. Acetone |
| 94a | reference | Biotechnol Bioeng. 2004 | Mannose, | was added to precipitate exopolysaccharide. |
| | | Feb 5; 85(3): 306-10 | Galacturonic | |
| | | | acid, | |
| | | | Arabinose, | |
| | | | and Ribose | |
| Anabaenaopsis | 1402/1⁹ | David KA, Fay P. Appl | unknown | See cited reference |
| circularis | | Environ Microbiol. 1977 | | |
| | | Dec; 34(6): 640-6 | | |
| Aphanocapsa | MN-11 | Sudo H., et al., Current | Rhamnose; | Cultured aerobically for 20 days in seawater-based |
| halophtia | | Micrcobiology Vol. 30 | mannose; fucose; | medium, with 8% NaCl, and 40 mg/L NaHPO4. |
| | | (1995), pp. 219-222 | galactose; | Nitrate changed the Exopolysaccharide content. |
| | | | xylose; | Highest cell density was obtained from culture |
| | | | glucose In | supplemented with 100 mg/l NaNO₃. Phosphorous |
| | | | ratio of | (40 mg/L) could be added to control the biomass |
| | | | :15:53:3:3:25 | and exopolysaccharide concentration. |
| Aphanocapsa sp | See reference | De Philippis R et al., Sci | unknown | Incubated at 20 and 28° C. with artificial light at a |
| | | Total Environ. 2005 Nov 2; | | photon flux of 5-20 umol m⁻² s⁻¹. |
| Cylindrotheca sp | See reference | De Philippis R et al., Sci | Glucuronic | Stock enriched cultures incubated at 20 and 28° C. |
| | | Total Environ. 2005 Nov 2; | acid, | with artificial light at a photon flux of 5-20 umol |
| | | | Galacturonic | m-2 s-1. Exopolysaccharide production done in |
| | | | acid, Glucose, | glass tubes containing 100 mL culture at 28° C. with |
| | | | Mannose, | continuous illumination at photon density of 5-10 |
| | | | Arabinose, | uE m-2 s-1. |
| | | | Fructose and | |
| | | | Rhamnose | |
| Navicula sp | See reference | De Philippis R et al., Sci | Glucuronic | Incubated at 20 and 28° C. with artificial light at a |
| | | Total Environ. 2005 Nov 2; | acid, | photon flux of 5-20 umol m-2 s-1. EPS production |
| | | | Galacturonic | done in glass tubes containing 100 mL culture at |
| | | | acid, Glucose, | 28° C. with continuous illumination at photon |
| | | | Mannose, | density of 5-10 uE m-2 s-1. |
| | | | Arabinose, | |
| | | | Fructose and | |
| | | | Rhamnose | |
| Gloeocapsa sp | See reference | De Philippis R et al., Sci | unknown | Incubated at 20 and 28° C. with artifical light at a |
| | | Total Environ. 2005 Nov 2; | | photon flux of 5-20 umol m-2 s-1. |
| Leptolyngbya sp | See reference | De Philippis R et al., Sci | unknown | Incubated at 20 and 28° C. with artificial light at a |
| | | Total Environ. 2005 Nov 2; | | photon flux of 5-20 umol m-2 s-1. |
| Symploca sp. | See reference | De Philippis R et al., Sci | unknown | Incubated at 20 and 28° C. with artificial light at a |
| | | Total Environ. 2005 Nov 2; | | photon flux of 5-20 umol m-2 s-1. |
| Synechocystis | PCC 6714/6803 | Jurgens UJ, Weckesser J. | Glucoseamine, | Photoautotrophically grown in BG-11 medium, pH |
| | | J Bacteriol. 1986 | mannosamine, | 7.5 at 25° C. Mass cultures prepared in a 12 liter |
| | | Nov; 168(2): 568-73 | galactosamine, | fermentor and gassed by air and carbon dioxide at |
| | | | mannose and | flow rates of 250 and 2.5 liters/h, with illumination |
| | | | glucose | from white fluorescent lamps at a constant light |
| | | | | intensity of 5,000 lux. |
| Stauroneis | See reference | Lind, JL (1997) Planta | unknown | See cited reference |
| decipiens | | 203: 213-221 | | |
| Achnanthes | Indiana | Holdsworth, RH., Cell | unknown | See cited reference |
| brevipes | University | Biol. 1968 Jun; 37(3): 831-7 | | |
| | Culture | | | |
| | Collection | | | |
| Achnanthes | Strain 330 from | Wang, Y., et al., Plant | unknown | See cited reference |
| longipes | National Institute | Physiol. 1997 | | |
| | for | Apr; 113(4): 1071-1080. | | |
| | Environmental | | | |
| | Studies | | | |

Microalgae are preferably cultured in liquid media for polysaccharide production. Culture condition parameters can be manipulated to optimize total polysaccharide production as well as to alter the structure of polysaccharides procduced by microalgae.

Microalgal culture media usually contains components such as a fixed nitrogen source, trace elements, a buffer for pH maintenance, and phosphate. Other components can include a fixed carbon source such as acetate or glucose, and salts such as sodium chloride, particularly for seawater microalgae. Examples of trace elements include zinc, boron, cobalt, copper, manganese, and molybdenum in, for example, the respective forms of ZnCl₂, H₃BO₃, CoCl₂.6H₂O, CuCl₂.2H₂O, MnCl₂.4H₂O and (NH₄)₆Mo₇O₂₄.4H₂O.

Some microalgae species can grow by utilizing a fixed carbon source such as glucose or acetate. Such microalgae can be cultured in bioreactors that do not allow light to enter. Alternatively, such microalgae can also be cultured in photobioreactors that contain the fixed carbon source and allow light to strike the cells. Such growth is known as heterotrophic growth. Any strain of microalgae, including those listed in Table 1, can be cultured in the presence of any one or more fixed carbon source including those listed in Tables 2 and 3.

TABLE 2

2,3-Butanediol
2-Aminoethanol
2′-Deoxy Adenosine
3-Methyl Glucose
Acetic Acid
Adenosine
Adenosine-5′-Monophosphate
Adonitol
Amygdalin
Arbutin
Bromosuccinic Acid
Cis-Aconitic Acid
Citric Acid
D,L-Carnitine
D,L-Lactic Acid
D,L-α-Glycerol Phosphate
D-Alanine
D-Arabitol
D-Cellobiose
Dextrin
D-Fructose
D-Fructose-6-Phosphate
D-Galactonic Acid Lactone
D-Galactose
D-Galacturonic Acid
D-Gluconic Acid
D-Glucosaminic Acid
D-Glucose-6-Phosphate
D-Glucuronic Acid
D-Lactic Acid Methyl Ester
D-L-α-Glycerol Phosphate
D-Malic Acid
D-Mannitol
D-Mannose
D-Melezitose
D-Melibiose
D-Psicose
D-Raffinose
D-Ribose
D-Saccharic Acid
D-Serine
D-Sorbitol
D-Tagatose
D-Trehalose
D-Xylose
Formic Acid
Gentiobiose
Glucuronamide
Glycerol
Glycogen
Glycyl-LAspartic Acid
Glycyl-LGlutamic Acid
Hydroxy-LProline
i-Erythritol
Inosine
Inulin
Itaconic Acid
Lactamide
Lactulose
L-Alaninamide
L-Alanine
L-Alanylglycine
L-Alanyl-Glycine
L-Arabinose
L-Asparagine
L-Aspartic Acid
L-Fucose
L-Glutamic Acid
L-Histidine
L-Lactic Acid
L-Leucine
L-Malic Acid
L-Ornithine
LPhenylalanine
L-Proline
L-Pyroglutamic Acid
L-Rhamnose
L-Serine
L-Threonine
Malonic Acid
Maltose
Maltotriose
Mannan
m-Inositol
N-Acetyl-DGalactosamine
N-Acetyl-DGlucosamine
N-Acetyl-LGlutamic Acid
N-Acetyl-β-DMannosamine
Palatinose
Phenyethylamine
p-Hydroxy-Phenylacetic Acid
Propionic Acid
Putrescine
Pyruvic Acid
Pyruvic Acid Methyl Ester
Quinic Acid
Salicin
Sebacic Acid
Sedoheptulosan
Stachyose
Succinamic Acid
Succinic Acid
Succinic Acid Mono-Methyl-Ester
Sucrose
Thymidine
Thymidine-5′-Monophosphate
Turanose
Tween 40
Tween 80
Uridine
Uridine-5′-Monophosphate
Urocanic Acid
Water
Xylitol
α-Cyclodextrin
α-D-Glucose
α-D-Glucose-1-Phosphate
α-D-Lactose
α-Hydroxybutyric Acid
α-Keto Butyric Acid
α-Keto Glutaric Acid
α-Keto Valeric Acid
α-Ketoglutaric Acid
α-Ketovaleric Acid
α-Methyl-DGalactoside
α-Methyl-DGlucoside
α-Methyl-DMannoside
β-Cyclodextrin
β-Hydroxybutyric Acid
β-Methyl-DGalactoside
β-Methyl-D-Glucoside
γ-Amino Butyric Acid
γ-Hydroxybutyric Acid

TABLE 3
(2-amino-3,4-dihydroxy-5-hydroxymethyl-1-cyclohexyl)glucopyranoside
(3,4-disinapoyl)fructofuranosyl-(6-sinapoyl)glucopyranoside
(3-sinapoyl)fructofuranosyl-(6-sinapoyl)glucopyranoside
1 reference
1,10-di-O-(2-acetamido-2-deoxyglucopyranosyl)-2-azi-1,10-decanediol
1,3-mannosylmannose
1,6-anhydrolactose
1,6-anhydrolactose hexaacetate
1,6-dichlorosucrose
1-chlorosucrose
1-desoxy-1-glycinomaltose
1-O-alpha-2-acetamido-2-deoxygalactopyranosyl-inositol
1-O-methyl-di-N-trifluoroacetyl-beta-chitobioside
1-propyl-4-O-beta galactopyranosyl-alpha galactopyranoside
2-(acetylamino)-4-O-(2-(acetylamino)-2-deoxy-4-O-sulfogalactopyranosyl)-2-deoxyglucose
2-(trimethylsilyl)ethyl lactoside
2,1′,3′,4′,6′-penta-O-acetylsucrose
2,2′-O-(2,2′-diacetamido-2,3,2′,3′-tetradeoxy-6,6′-di-O-(2-tetradecylhexadecanoyl)-
alpha,alpha′-trehalose-3,3′-diyl)bis(N-lactoyl-alanyl-isoglutamine)
2,3,6,2′,3′,4′,6′-hepta-O-acetylcellobiose
2,3′-anhydrosucrose
2,3-di-O-phytanyl-1-O-(mannopyranosyl-(2-sulfate)-(1-2)-glucopyranosyl)-sn-glycerol
2,3-epoxypropyl O-galactopyranosyl(1-6)galactopyranoside
2,3-isoprolylideneerthrofuranosyl 2,3-O-isopropylideneerythrofuranoside
2′,4′-dinitrophenyl 2-deoxy-2-fluoro-beta-xylobioside
2,5-anhydromannitol iduronate
2,6-sialyllactose
2-acetamido-2,4-dideoxy-4-fluoro-3-O-galactopyranosylglucopyranose
2-acetamido-2-deoxy-3-O-(gluco-4-enepyranosyluronic acid)glucose
2-acetamido-2-deoxy-3-O-rhamnopyranosylglucose
2-acetamido-2-deoxy-6-O-beta galactopyranosylgalactopyranose
2-acetamido-2-deoxyglucosylgalactitol
2-acetamido-3-O-(3-acetamido-3,6-dideoxy-beta-glucopyranosyl)-2-deoxy-galactopyranose
2-amino-6-O-(2-amino-2-deoxy-glucopyranosyl)-2-deoxyglucose
2-azido-2-deoxymannopyranosyl-(1,4)-rhamnopyranose
2-deoxy-6-O-(2,3-dideoxy-4,6-O-isopropylidene-2,3-(N-tosylepimino)mannopyranosyl)-4,5-
O-isopropylidene-1,3-di-N-tosylstreptamine
2-deoxymaltose
2-iodobenzyl-1-thiocellobioside
2-N-(4-benzoyl)benzoyl-1,3-bis(mannos-4-yloxy)-2-propylamine
2-nitrophenyl-2-acetamido-2-deoxy-6-O-beta galactopyranosyl-alpha galactopyranoside
2-O-(glucopyranosyluronic acid)xylose
2-O-glucopyranosylribitol-1-phosphate
2-O-glucopyranosylribitol-4′-phosphate
2-O-rhamnopyranosyl-rhamnopyranosyl-3-hydroxyldecanoyl-3-hydroxydecanoate
2-O-talopyranosylmannopyranoside
2-thiokojibiose
2-thiosophorose
3,3′-neotrehalosadiamine
3,6,3′,6′-dianhydro(galactopyranosylgalactopyranoside)
3,6-di-O-methyl-beta-glucopyranosyl-(1-4)-2,3-di-O-methyl-alpha-rhamnopyranose
3-amino-3-deoxyaltropyranosyl-3-amino-3-deoxyaltropyranoside
3-deoxy-3-fluorosucrose
3-deoxy-5-O-rhamnopyranosyl-2-octulopyranosonate
3-deoxyoctulosonic acid-(alpha-2-4)-3-deoxyoctulosonic acid
3-deoxysucrose
3-ketolactose
3-ketosucrose
3-ketotrehalose
3-methyllactose
3-O-(2-acetamido-6-O-(N-acetylneuraminyl)-2-deoxygalactosyl)serine
3-O-(glucopyranosyluronic acid)galactopyranose
3-O-beta-glucuronosylgalactose
3-O-fucopyranosyl-2-acetamido-2-deoxyglucopyranose
3′-O-galactopyranosyl-1-4-O-galactopyranosylcytarabine
3-O-galactosylarabinose
3-O-talopyranosylmannopyranoside
3-trehalosamine
4-(trifluoroacetamido)phenyl-2-acetamido-2-deoxy-4-O-beta-mannopyranosyl-beta-
glucopyranoside
4,4′,6,6′-tetrachloro-4,4′,6,6′-tetradeoxygalactotrehalose
4,6,4′,6′-dianhydro(galactopyranosylgalactopyranoside)
4,6-dideoxysucrose
4,6-O-(1-ethoxy-2-propenylidene)sucrose hexaacetate
4-chloro-4-deoxy-alpha-galactopyranosyl 3,4-anhydro-1,6-dichloro-1,6-dideoxy-beta-lyxo-
hexulofuranoside
4-glucopyranosylmannose
4-methylumbelliferylcellobioside
4-nitrophenyl 2-fucopyranosyl-fucopyranoside
4-nitrophenyl 2-O-alpha-D-galactopyranosyl-alpha-D-mannopyranoside
4-nitrophenyl 2-O-alpha-D-glucopyranosyl-alpha-D-mannopyranoside
4-nitrophenyl 2-O-alpha-D-mannopyranosyl-alpha-D-mannopyranoside
4-nitrophenyl 6-O-alpha-D-mannopyranosyl-alpha-D-mannopyranoside
4-nitrophenyl-2-acetamido-2-deoxy-6-O-beta-D-galactopyranosyl-beta-D-glucopyranoside
4-O-(2-acetamido-2-deoxy-beta-glucopyranosyl)ribitol
4-O-(2-amino-2-deoxy-alpha-glucopyranosyl)-3-deoxy-manno-2-octulosonic acid
4-O-(glucopyranosyluronic acid)xylose
4-O-acetyl-alpha-N-acetylneuraminyl-(2-3)-lactose
4-O-alpha-D-galactopyranosyl-D-galactose
4-O-galactopyranosyl-3,6-anhydrogalactose dimethylacetal
4-O-galactopyranosylxylose
4-O-mannopyranosyl-2-acetamido-2-deoxyglucose
4-thioxylobiose
4-trehalosamine
4-trifluoroacetamidophenyl 2-acetamido-4-O-(2-acetamido-2-deoxyglucopyranosyl)-2-
deoxymannopyranosiduronic acid
5-bromoindoxyl-beta-cellobioside
5′-O-(fructofuranosyl-2-1-fructofuranosyl)pyridoxine
5-O-beta-galactofuranosyl-galactofuranose
6 beta-galactinol
6(2)-thiopanose
6,6′-di-O-corynomycoloyl-alpha-mannopyranosyl-alpha-mannopyranoside
6,6-di-O-maltosyl-beta-cyclodextrin
6,6′-di-O-mycoloyl-alpha-mannopyranosyl-alpha-mannopyranoside
6-chloro-6-deoxysucrose
6-deoxy-6-fluorosucrose
6-deoxy-alpha-gluco-pyranosiduronic acid
6-deoxy-gluco-heptopyranosyl 6-deoxy-gluco-heptopyranoside
6-deoxysucrose
6-O-decanoyl-3,4-di-O-isobutyrylsucrose
6-O-galactopyranosyl-2-acetamido-2-deoxygalactose
6-O-galactopyranosylgalactose
6-O-heptopyranosylglucopyranose
6-thiosucrose
7-O-(2-amino-2-deoxyglucopyranosyl)heptose
8-methoxycarbonyloctyl-3-O-glucopyranosyl-mannopyranoside
8-O-(4-amino-4-deoxyarabinopyranosyl)-3-deoxyoctulosonic acid
allolactose
allosucrose
allyl 6-O-(3-deoxyoct-2-ulopyranosylonic acid)-(1-6)-2-deoxy-2-(3-
hydroxytetradecanamido)glucopyranoside 4-phosphate
alpha-(2-9)-disialic acid
alpha,alpha-trehalose 6,6′-diphosphate
alpha-glucopyranosyl alpha-xylopyranoside
alpha-maltosyl fluoride
aprosulate
benzyl 2-acetamido-2-deoxy-3-O-(2-O-methyl-beta-galactosyl)-beta-glucopyranoside
benzyl 2-acetamido-2-deoxy-3-O-beta fucopyranosyl-alpha-galactopyranoside
benzyl 2-acetamido-6-O-(2-acetamido-2,4-dideoxy-4-fluoroglucopyranosyl)-2-
deoxygalactopyranoside
benzyl gentiobioside
beta-D-galactosyl(1-3)-4-nitrophenyl-N-acetyl-alpha-D-galactosamine
beta-methylmelibiose
calcium sucrose phosphate
camiglibose
cellobial
cellobionic acid
cellobionolactone
Cellobiose
cellobiose octaacetate
cellobiosyl bromide heptaacetate
Celsior
chitobiose
chondrosine
Cristolax
deuterated methyl beta-mannobioside
dextrin maltose
D-glucopyranose, O-D-glucopyranosyl
Dietary Sucrose
difructose anhydride I
difructose anhydride III
difructose anhydride IV
digalacturonic acid
DT 5461
ediol
epilactose
epsilon-N-1-(1-deoxylactulosyl)lysine
feruloyl arabinobiose
floridoside
fructofuranosyl-(2-6)-glucopyranoside
FZ 560
galactosyl-(1-3)galactose
garamine
gentiobiose
geranyl 6-O-alpha-L-arabinopyranosyl-beta-D-glucopyranoside
geranyl 6-O-xylopyranosyl-glucopyranoside
glucosaminyl-1,6-inositol-1,2-cyclic monophosphate
glucosyl (1-4) N-acetylglucosamine
glucuronosyl(1-4)-rhamnose
heptosyl-2-keto-3-deoxyoctonate
inulobiose
Isomaltose
isomaltulose
isoprimeverose
kojibiose
lactobionic acid
lacto-N-biose II
Lactose
lactosylurea
Lactulose
laminaribiose
lepidimoide
leucrose
levanbiose
lucidin 3-O-beta-primveroside
LW 10121
LW 10125
LW 10244
maltal
maltitol
Maltose
maltose hexastearate
maltose-maleimide
maltosylnitromethane heptaacetate
maltosyltriethoxycholesterol
maltotetraose
Malun 25
mannosucrose
mannosyl-(1-4)-N-acetylglucosaminyl-(1-N)-urea
mannosyl(2)-N-acetyl(2)-glucose
melibionic acid
Melibiose
melibiouronic acid
methyl 2-acetamido-2-deoxy-3-O-(alpha-idopyranosyluronic acid)-4-O-sulfo-beta-
galactopyranoside
methyl 2-acetamido-2-deoxy-3-O-(beta-glucopyranosyluronic acid)-4-O-sulfo-beta-
galactopyranoside
methyl 2-acetamido-2-deoxy-3-O-glucopyranosyluronoylglucopyranoside
methyl 2-O-alpha-rhamnopyranosyl-beta-galactopyranoside
methyl 2-O-beta-rhamnopyranosyl-beta-galactopyranoside
methyl 2-O-fucopyranosylfucopyranoside 3 sulfate
methyl 2-O-mannopyranosylmannopyranoside
methyl 2-O-mannopyranosyl-rhamnopyranoside
methyl 3-O-(2-acetamido-2-deoxy-6-thioglucopyranosyl)galactopyranoside
methyl 3-O-galactopyranosylmannopyranoside
methyl 3-O-mannopyranosylmannopyranoside
methyl 3-O-mannopyranosyltalopyranoside
methyl 3-O-talopyranosyltalopyranoside
methyl 4-O-(6-deoxy-manno-heptopyranosyl)galactopyranoside
methyl 6-O-acetyl-3-O-benzoyl-4-O-(2,3,4,6-tetra-O-benzoylgalactopyranosyl)-2-deoxy-2-
phthalimidoglucopyranoside
methyl 6-O-mannopyranosylmannopyranoside
methyl beta-xylobioside
methyl fucopyranosyl(1-4)-2-acetamido-2-deoxyglucopyranoside
methyl laminarabioside
methyl O-(3-deoxy-3-fluorogalactopyranosyl)(1-6)galactopyranoside
methyl-2-acetamido-2-deoxyglucopyranosyl-1-4-glucopyranosiduronic acid
methyl-2-O-fucopyranosylfucopyranoside 4-sulfate
MK 458
N(1)-2-carboxy-4,6-dinitrophenyl-N(6)-lactobionoyl-1,6-hexanediamine
N-(2,4-dinitro-5-fluorophenyl)-1,2-bis(mannos-4′-yloxy)propyl-2-amine
N-(2′-mercaptoethyl)lactamine
N-(2-nitro-4-azophenyl)-1,3-bis(mannos-4′-yloxy)propyl-2-amine
N-(4-azidosalicylamide)-1,2-bis(mannos-4′-yloxy)propyl-2-amine
N,N-diacetylchitobiose
N-acetylchondrosine
N-acetyldermosine
N-acetylgalactosaminyl-(1-4)-galactose
N-acetylgalactosaminyl-(1-4)-glucose
N-acetylgalactosaminyl-1-4-N-acetylglucosamine
N-acetylgalactosaminyl-1-4-N-acetylglucosamine
N-acetylgalactosaminyl-alpha(1-3)galactose
N-acetylglucosamine-N-acetylmuramyl-alanyl-isoglutaminyl-alanyl-glycerol dipalmitoyl
N-acetylglucosaminyl beta(1-6)N-acetylgalactosamine
N-acetylglucosaminyl-1-2-mannopyranose
N-acetylhyalobiuronic acid
N-acetylneuraminoyllactose
N-acetylneuraminoyllactose sulfate ester
N-acetylneuraminyl-(2-3)-galactose
N-acetylneuraminyl-(2-6)-galactose
N-benzyl-4-O-(beta-galactopyranosyl)glucamine-N-carbodithioate
neoagarobiose
N-formylkansosaminyl-(1-3)-2-O-methylrhamnopyranose
O-((Nalpha)-acetylglucosamine 6-sulfate)-(1-3)-idonic acid
O-(4-O-feruloyl-alpha-xylopyranosyl)-(1-6)-glucopyranose
O-(alpha-idopyranosyluronic acid)-(1-3)-2,5-anhydroalditol-4-sulfate
O-(glucuronic acid 2-sulfate)-(1-3)-O-(2,5)-andydrotalitol 6-sulfate
O-(glucuronic acid 2-sulfate)-(1-4)-O-(2,5)-anhydromannitol 6-sulfate
O-alpha-glucopyranosyluronate-(1-2)-galactose
O-beta-galactopyranosyl-(1-4)-O-beta-xylopyranosyl-(1-0)-serine
octyl maltopyranoside
O-demethylpaulomycin A
O-demethylpaulomycin B
O-methyl-di-N-acetyl beta-chitobioside
Palatinit
paldimycin
paulomenol A
paulomenol B
paulomycin A
paulomycin A2
paulomycin B
paulomycin C
paulomycin D
paulomycin E
paulomycin F
phenyl 2-acetamido-2-deoxy-3-O-beta-D-galactopyranosyl-alpha-D-galactopyranoside
phenyl O-(2,3,4,6-tetra-O-acetylgalactopyranosyl)-(1-3)-4,6-di-O-acetyl-2-deoxy-2-
phthalimido-1-thioglucopyranoside
poly-N-4-vinylbenzyllactonamide
pseudo-cellobiose
pseudo-maltose
rhamnopyranosyl-(1-2)-rhamnopyranoside-(1-methyl ether)
rhoifolin
ruberythric acid
S-3105
senfolomycin A
senfolomycin B
solabiose
SS 554
streptobiosamine
Sucralfate
Sucrose
sucrose acetate isobutyrate
sucrose caproate
sucrose distearate
sucrose monolaurate
sucrose monopalmitate
sucrose monostearate
sucrose myristate
sucrose octaacetate
sucrose octabenzoic acid
sucrose octaisobutyrate
sucrose octasulfate
sucrose polyester
sucrose sulfate
swertiamacroside
T-1266
tangshenoside I
tetrahydro-2-((tetrahydro-2-furanyl)oxy)-2H-pyran
thionigerose
Trehalose
trehalose 2-sulfate
trehalose 6,6′-dipalmitate
trehalose-6-phosphate
trehalulose
trehazolin
trichlorosucrose
tunicamine
turanose
U 77802
U 77803
xylobiose
xylose-glucose
xylosucrose

Microalgae contain photosynthetic machinery capable of metabolizing photons, and transferring energy harvested from photons into fixed chemical energy sources such as monosaccharide. Glucose is a common monosaccharide produced by microalgae by metabolizing light energy and fixing carbon from carbon dioxide. Some microalgae can also grow in the absence of light on a fixed carbon source that is exogenously provided (for example see Plant Physiol. 2005 February; 137(2):460-74). In addition to being a source of chemical energy, monosaccharides such as glucose are also substrate for production of polysaccharides (see Example 14). The invention provides methods of producing polysaccharides with novel monosaccharide compositions. For example, microalgae is cultured in the presence of culture media that contains exogenously provided monosaccharide, such as glucose. The monosaccharide is taken up by the cell by either active or passive transport and incorporated into polysaccharide molecules produced by the cell. This novel method of polysaccharide composition manipulation can be performed with, for example, any microalgae listed in Table 1 using any monosaccharide or disaccharide listed in Tables 2 or 3.

In some embodiments, the fixed carbon source is one or more selected from glucose, galactose, xylose, mannose, rhamnose, N-acetylglucosamine, glycerol, floridoside, and glucuronic acid. The methods may be practiced cell growth in the presence of at least about 5.0 μM, at least about 10 μM, at least about 15.0 μM, at least about 20.0 μM, at least about 25.0 μM, at least about 30.0 μM, at least about 35.0 μM, at least about 40.0 μM, at least about 45.0 μM, at least about 50.0 μM, at least about 55.0 μM, at least about 60.0 μM, at least about 75.0 μM, at least about 80.0 μM, at least about 85.0 μM, at least about 90.0 μM, at least about 95.0 μM, at least about 100.0 μM, or at least about 150.0 μM, of one or more exogenously provided fixed carbon sources selected from Tables 2 and 3.

In some embodiments using cells of the genus Porphyridium, the methods include the use of approximately 0.5-0.75% glycerol as a fixed carbon source when the cells are cultured in the presence of light. Alternatively, a range of glycerol, from approximately 4.0% to approximately 9.0% may be used when the Porphyridium cells are cultured in the dark, more preferably from 5.0% to 8.0%, and more preferably 7.0%.

After culturing the microalgae in the presence of the exogenously provided carbon source, the monosaccharide composition of the polysaccharide can be analyzed as described in Example 5. Microalgae can be transformed with genes encoding carbohydrate transporters to facilitate the uptake of exogenously provided carbohydrates such SEQ ID NOs: 13, 15, 17, 19 and 20.

Microalgae culture media can contain a fixed nitrogen source such as KNO₃. Alternatively, microalgae are placed in culture conditions that do not include a fixed nitrogen source. For example, Prophridium sp. cells are cultured for a first period of time in the presence of a fixed nitrogen source, and then the cells are cultured in the absence of a fixed nitrogen source (see for example Adda M., Biomass 10:131-140. (1986); Sudo H., et al., Current Microbiology Vol. 30 (1995), pp. 219-222; Marinho-Soriano E., Bioresour Technol. 2005 February; 96(3):379-82; Bioresour. Technol. 42:141-147 (1992)).

Other culture parameters can also be manipulated, such as the pH of the culture media, the identity and concentration of trace elements such as those listed in Table 3, and other media constituents.

Microalgae can be grown in the presence of light. The number of photons striking a culture of microalgae cells can be manipulated, as well as other parameters such as the wavelength spectrum and ratio of dark:light hours per day. Microalgae can also be cultured in natural light, as well as simultaneous and/or alternating combinations of natural light and artificial light. For example, microalgae of the genus Chlorella be cultured under natural light during daylight hours and under artificial light during night hours.

The gas content of a photobioreactor can be manipulated. Part of the volume of a photobioreactor can contain gas rather than liquid. Gas inlets can be used to pump gases into the photobioreactor. Any gas can be pumped into a photobioreactor, including air, air/CO₂ mixtures, noble gases such as argon and others. The rate of entry of gas into a photobioreactor can also be manipulated. Increasing gas flow into a photobioreactor increases the turbidity of a culture of microalgae. Placement of ports conveying gases into a photobioreactor can also affect the turbidity of a culture at a given gas flow rate. Air/CO₂ mixtures can be modulated to generate different polysaccharide compositions by manipulating carbon flux. For example, air:CO₂ mixtures of about 99.75% air:0.25% CO₂, about 99.5% air:0.5% CO₂, about 99.0% air:1.00% CO₂, about 98.0% air:2.0% CO₂, about 97.0% air:3.0% CO₂, about 96.0% air:4.0% CO₂, and about 95.00% air:5.0% CO₂ can be infused into a bioreactor or photobioreactor.

Microalgae cultures can also be subjected to mixing using devices such as spinning blades and propellers, rocking of a culture, stir bars, and other instruments.

B. Cell Culture Methods: Photobioreactors

Microalgae can be grown and maintained in closed photobioreactors made of different types of transparent or semitransparent material. Such material can include Plexiglas® enclosures, glass enclosures, bags bade from substances such as polyethylene, transparent or semitransparent pipes, and other materials. Microalgae can also be grown in open ponds.

Photobioreactors can have ports allowing entry of gases, solids, semisolids and liquids into the chamber containing the microalgae. Ports are usually attached to tubing or other means of conveying substances. Gas ports, for example, convey gases into the culture. Pumping gases into a photobioreactor can serve to both feed cells CO₂ and other gases and to aerate the culture and therefore generate turbidity. The amount of turbidity of a culture varies as the number and position of gas ports is altered. For example, gas ports can be placed along the bottom of a cylindrical polyethylene bag. Microalgae grow faster when CO₂ is added to air and bubbled into a photobioreactor. For example, a 5% CO₂:95% air mixture is infused into a photobioreactor containing cells of the genus Porphyridium (see for example Biotechnol Bioeng. 1998 Sep. 20; 59(6):705-13; textbook from office; U.S. Pat. Nos. 5,643,585 and 5,534,417; Lebeau, T., et. al. Appl. Microbiol Biotechnol (2003) 60:612-623; Muller-Fuega, A., Journal of Biotechnology 103 (2003 153-163; Muller-Fuega, A., Biotechnology and Bioengineering, Vol. 84, No. 5, Dec. 5, 2003; Garcia-Sanchez, J. L., Biotechnology and Bioengineering, Vol. 84, No. 5, Dec. 5, 2003; Garcia-Gonzales, M., Journal of Biotechnology, 115 (2005) 81-90. Molina Grima, E., Biotechnology Advances 20 (2003) 491-515).

Photobioreactors can be exposed to one or more light sources to provide microalgae with light as an energy source via light directed to a surface of the photobioreactor. Preferably the light source provides an intensity that is sufficient for the cells to grow, but not so intense as to cause oxidative damage or cause a photoinhibitive response. In some instances a light source has a wavelength range that mimics or approximately mimics the range of the sun. In other instances a different wavelength range is used. Photobioreactors can be placed outdoors or in a greenhouse or other facility that allows sunlight to strike the surface. Preferred photon intensities for species of the genus Porphyridium are between 50 and 300 uE m⁻² s⁻¹ (see for example Photosynth Res. 2005 June; 84(1-3):21-7).

Photobioreactor preferably have one or more parts that allow media entry. It is not necessary that only one substance enter or leave a port. For example, a port can be used to flow culture media into the photobioreactor and then later can be used for sampling, gas entry, gas exit, or other purposes. In some instances a photobioreactor is filled with culture media at the beginning of a culture and no more growth media is infused after the culture is inoculated. In other words, the microalgal biomass is cultured in an aqueous medium for a period of time during which the microalgae reproduce and increase in number; however quantities of aqueous culture medium are not flowed through the photobioreactor throughout the time period. Thus in some embodiments, aqueous culture medium is not flowed through the photobioreactor after inoculation.

In other instances culture media can be flowed though the photobioreactor throughout the time period during which the microalgae reproduce and increase in number. In some instances media is infused into the photobioreactor after inoculation but before the cells reach a desired density. In other words, a turbulent flow regime of gas entry and media entry is not maintained for reproduction of microalgae until a desired increase in number of said microalgae has been achieved, but instead a parameter such as gas entry or media entry is altered before the cells reach a desired density.

Photobioreactors preferably have one or more ports that allow gas entry. Gas can serve to both provide nutrients such as CO₂ as well as to provide turbulence in the culture media. Turbulence can be achieved by placing a gas entry port below the level of the aqueous culture media so that gas entering the photobioreactor bubbles to the surface of the culture. One or more gas exit ports allow gas to escape, thereby preventing pressure buildup in the photobioreactor. Preferably a gas exit port leads to a “one-way” valve that prevents contaminating microorganisms to enter the photobioreactor. In some instances cells are cultured in a photobioreactor for a period of time during which the microalgae reproduce and increase in number, however a turbulent flow regime with turbulent eddies predominantly throughout the culture media caused by gas entry is not maintained for all of the period of time. In other instances a turbulent flow regime with turbulent eddies predominantly throughout the culture media caused by gas entry can be maintained for all of the period of time during which the microalgae reproduce and increase in number. In some instances a predetermined range of ratios between the scale of the photobioreactor and the scale of eddies is not maintained for the period of time during which the microalgae reproduce and increase in number. In other instances such a range can be maintained.

Photobioreactors preferably have at least one port that can be used for sampling the culture. Preferably a sampling port can be used repeatedly without altering compromising the axenic nature of the culture. A sampling port can be configured with a valve or other device that allows the flow of sample to be stopped and started. Alternatively a sampling port can allow continuous sampling. Photobioreactors preferably have at least one port that allows inoculation of a culture. Such a port can also be used for other purposes such as media or gas entry.

Microalgae that produce polysaccharides can be cultured in photobioreactors. Microalgae that produce polysaccharide that is not attached to cells can be cultured for a period of time and then separated from the culture media and secreted polysaccharide by methods such as centrifugation and tangential flow filtration. Preferred organisms for culturing in photobioreactors to produce polysaccharides include Porphyridium sp., Porphyridium cruentum, Porphyridium purpureum, Porphyridium aerugineum, Rhodella maculata, Rhodella reticulata, Chlorella autotrophica, Chlorella stigmatophora, Chlorella capsulata, Achnanthes brevipes and Achnanthes longipes.

C. Non-Microalgal Polysaccharide Production

Organisms besides microalgae can be used to produce polysaccharides, such as lactic acid bacteria (see for example Stinglee, F., Molecular Microbiology (1999) 32(6), 1287-1295; Ruas_Madiedo, P., J. Dairy Sci. 88:843-856 (2005); Laws, A., Biotechnology Advances 19 (2001) 597-625; Xanthan gum bacteria: Pollock, T J., J. Ind. Microbiol Biotechnol., 1997 August; 19(2):92-7.; Becker, A., Appl. Micrbiol. Bioltechnol. 1998 August; 50(2):92-7; Garcia-Ochoa, F., Biotechnology Advances 18 (2000) 549-579., seaweed: Talarico, L B., et al., Antiviral Research 66 (2005) 103-110; Dussealt, J., et al., J Biomed Mater Res A., 2005 Nov. 1; Melo, F. R., J Biol Chem 279:20824-35 (2004)).

D. Ex Vivo Methods

Microalgae and other organisms can be manipulated to produce polysaccharide molecules that are not naturally produced by methods such as feeding cells with monosaccharides that are not produced by the cells (Nature. 2004 Aug. 19; 430(7002):873-7). For example, species listed in Table I are grown according to the referenced growth protocols, with the additional step of adding to the culture media a fixed carbon source that is not in the culture media as published and referenced in Table 1 and is not produced by the cells in a detectable amount. In addition, such cells can first be transformed to contain a carbohydrate transporter, thus facilitating the entry of monosaccharides.

E. In Vitro Methods

Polysaccharides can be altered by enzymatic and chemical modification. For example, carbohydrate modifying enzymes can be added to a preparation of polysaccharide and allowed to catalyze reactions that alter the structure of the polysaccharide. Chemical methods can be used to, for example, modify the sulfation pattern of a polysaccharide (see for example Carbohydr. Polym. 63:75-80 (2000); Pomin V H., Glycobiology. 2005 December; 15(12):1376-85; Naggi A., Semin Thromb Hemost. 2001 October; 27(5):437-43 Review; Habuchi, O., Glycobiology. 1996 January; 6(1);51-7; Chen, J., J. Biol. Chem. In press; Geresh., S et al., J. Biochem. Biophys. Methods 50 (2002) 179-187.).

F. Polysaccharide Purification Methods

Exopolysaccharides can be purified from microalgal cultures by various methods, including those disclosed herein.

Precipitation

For example, polysaccharides can be precipitated by adding compounds such as cetylpyridinium chloride, isopropanol, ethanol, or methanol to an aqueous solution containing a polysaccharide in solution. Pellets of precipitated polysaccharide can be washed and resuspended in water, buffers such as phosphate buffered saline or Tris, or other aqueous solutions (see for example Farias, W. R. L., et al., J. Biol. Chem. (2000) 275; (38)29299-29307; U.S. Pat. No. 6,342,367; U.S. Pat. No. 6,969,705).

Dialysis

Polysaccharides can also be dialyzed to remove excess salt and other small molecules (see for example Gloaguen, V., et al., Carbohydr Res. 2004 Jan. 2; 339(1):97-103; Microbiol Immunol. 2000; 44(5):395-400.).

Tangential Flow Filtration

Filtration can be used to concentrate polysaccharide and remove salts. For example, tangential flow filtration (TFF), also known as cross-flow filtration, can be used (see for example Millipore Pellicon® device, used with 1000 kD membrane (catalog number P2C01MC01); Geresh, Carb. Polym. 50; 183-189 (2002)). It is preferred that the polysaccharides do not pass through the filter at a significant level. It is also preferred that polysaccharides do not adhere to the filter material. TFF can also be performed using hollow fiber filtration systems.

Non-limiting examples of tangential flow filtration include use of a filter with a pore size of at least about 0.1 micrometer, at least about 0.12 micrometer, at least about 0.14 micrometer, at least about 0.16 micrometer, at least about 0.18 micrometer, at least about 0.2 micrometer, at least about 0.22 micrometer, or at least about 0.45 micrometer. Preferred pore sizes of TFF allow contaminants to pass through but not polysaccharide molecules.

Ion Exchange Chromatography

Anionic polysaccharides can be purified by anion exchange chromatography. (Jacobsson, I., Biochem J. 1979 Apr. 1; 179(1):77-89; Karamanos, N K., Eur J Biochem. 1992 Mar. 1; 204(2):553-60).

Protease Treatment

Polysaccharides can be treated with proteases to degrade contaminating proteins. In some instances the contaminating proteins are attached, either covalently or noncovalently to polysaccharides. In other instances the polysaccharide molecules are in a preparation that also contains proteins. Proteases can be added to polysaccharide preparations containing proteins to degrade proteins (for example, the protease from Streptomyces griseus can be used (SigmaAldrich catalog number P5147). After digestion, the polysaccharide is preferably purified from residual proteins, peptide fragments, and amino acids. This purification can be accomplished, for example, by methods listed above such as dialysis, filtration, and precipitation.

Heat treatment can also be used to eliminate proteins in polysaccharide preparations (see for example Biotechnol Lett. 2005 January; 27(1):13-8; FEMS Immunol Med Microbiol. 2004 Oct. 1; 42(2):155-66; Carbohydr Res. 2000 Sep. 8; 328(2):199-207; J Biomed Mater Res. 1999; 48(2):111-6; Carbohydr Res. 1990 Oct. 15; 207(1):101-20;).

The invention thus includes production of an exopolysaccharide comprising separating the exopolysaccharide from contaminants after proteins attached to the exopolysaccharide have been degraded or destroyed. The proteins may be those attached to the exopolysaccharide during culture of a microalgal cell in media, which is first separated from the cells prior to proteolysis or protease treatment. The cells may be those of the genus Porphyridium as a non-limiting example.

In one non-limiting example, a method of producing an exopolysaccharide is provided wherein the method comprises culturing cells of the genus Porphyridium; separating cells from culture media; destroying protein attached to the exopolysaccharide present in the culture media; and separating the exopolysaccharide from contaminants. In some methods, the contaminant(s) are selected from amino acids, peptides, proteases, protein fragments, and salts. In other methods, the contaminant is selected from NaCl, MgSO₄, MgCl₂, CaCl₂, KNO₃, KH₂PO₄, NaHCO₃, Tris, ZnCl₂, H₃BO₃, CoCl₂, CuCl₂, MnCl₂, (NH₄)₆Mo₇O₂₄, FeCl3 and EDTA.

Drying Methods

After purification of methods such as those above, polysaccharides can be dried using methods such as lyophilization and heat drying (see for example Shastry, S., Brazilian Journal of Microbiology (2005) 36:57-62; Matthews K H., Int J Pharm. 2005 Jan. 31; 289(1-2):51-62. Epub 2004 Dec. 30; Gloaguen, V., et al., Carbohydr Res. 2004 Jan. 2; 339(1):97-103).

Tray dryers accept moist solid on trays. Hot air (or nitrogen) is circulated to dry. Shelf dryers can also employ reduced pressure or vacuum to dry at room temperature when products are temperature sensitive and are similar to a freeze-drier but less costly to use and can be easily scaled-up.

Spray dryers are relatively simple in operation, which accept feed in fluid state and convert it into a dried particulate form by spraying the fluid into a hot drying medium.

Rotary dryers operate by continuously feeding wet material, which is dried by contact with heated air, while being transported along the interior of a rotating cylinder, with the rotating shell acting as the conveying device and stirrer.

Spin flash dryers are used for drying of wet cake, slurry, or paste which is normally difficult to dry in other dryers. The material is fed by a screw feeder through a variable speed drive into the vertical drying chamber where it is heated by air and at the same time disintegrated by a specially designed disintegrator. The heating of air may be direct or indirect depending upon the application. The dry powder is collected through a cyclone separator/bag filter or with a combination of both.

Whole Cell Extraction

Intracellular polysaccharides and cell wall polysaccharides can be purified from whole cell mass (see form example U.S. Pat. No. 4,992,540; U.S. Pat. No. 4,810,646; J Sietsma J H., et al., Gen Microbiol. 1981 July; 125(1):209-12; Fleet G H, Manners D J., J Gen Microbiol. 1976 May; 94(1):180-92).

G. Microalgae Homogenization Methods

A pressure disrupter pumps of a slurry through a restricted orifice valve. High pressure (up to 1500 bar) is applied, followed by an instant expansion through an exiting nozzle. Cell disruption is accomplished by three different mechanisms: impingement on the valve, high liquid shear in the orifice, and sudden pressure drop upon discharge, causing an explosion of the cell. The method is applied mainly for the release of intracellular molecules. According to Hetherington et al., cell disruption (and consequently the rate of protein release) is a first-order process, described by the relation: log[Rm/(Rm−R)]=K N P72.9. R is the amount of soluble protein; Rm is the maximum amount of soluble protein K is the temperature dependent rate constant; N is the number of passes through the homogenizer (which represents the residence time). P is the operating pressure.

In a ball mill, cells are agitated in suspension with small abrasive particles. Cells break because of shear forces, grinding between beads, and collisions with beads. The beads disrupt the cells to release biomolecules. The kinetics of biomolecule release by this method is also a first-order process.

Another widely applied method is the cell lysis with high frequency sound that is produced electronically and transported through a metallic tip to an appropriately concentrated cellular suspension, ie: sonication. The concept of ultrasonic disruption is based on the creation of cavities in cell suspension.

Blending (high speed or Waring), the french press, or even centrifugation in case of weak cell walls, also disrupt the cells by using the same concepts.

Cells can also be ground after drying in devices such as a colloid mill.

Because the percentage of polysaccharide as a function of the dry weight of a microalgae cell can frequently be in excess of 50%, microalgae cell homogenates can be considered partially pure polysaccharide compositions. Cell disruption aids in increasing the amount of solvent-accessible polysaccharide by breaking apart cell walls that are largely composed of polysaccharide.

H. Analysis Methods

Assays for detecting polysaccharides can be used to quantitate starting polysaccharide concentration, measure yield during purification, calculate density of secreted polysaccharide in a photobioreactor, measure polysaccharide concentration in a finished product, and other purposes.

The phenol: sulfuric acid assay detects carbohydrates (see Hellebust, Handbook of Phycological Methods, Cambridge University Press, 1978; and Cuesta G., et al., J Microbiol Methods. 2003 January; 52(1):69-73). The 1,6 dimethylmethylene blue assay detects anionic polysaccharides. (see for example Braz J Med Biol Res. 1999 May; 32(5):545-50; Clin Chem. 1986 November; 32(11):2073-6).

Polysaccharides can also be analyzed through methods such as HPLC, size exclusion chromatography, and anion exchange chromatography (see for example Prosky L, Asp N, Schweizer T F, DeVries J W & Furda I (1988) Determination of insoluble, soluble and total dietary fiber in food and food products: Interlaboratory study. Journal of the Association of Official Analytical Chemists 71, 1017±1023; Int J Biol Macromol. 2003 November; 33(1-3):9-18)

Polysaccharides can also be detected using gel electrophoresis (see for example Anal Biochem. 2003 Oct. 15; 321(2):174-82; Anal Biochem. 2002 Jan. 1; 300(1):53-68).

Monosaccharide analysis of polysaccharides can be performed by combined gas chromatography/mass spectrometry (GC/MS) of the per-O-trimethylsilyl (TMS) derivatives of the monosaccharide methyl glycosides produced from the sample by acidic methanolysis (see Merkle and Poppe (1994) Methods Enzymol. 230: 1-15; York, et al. (1985) Methods Enzymol. 118:3-40).

III Compositions

A. General

Compositions of the invention include a microalgal polysaccharide or homogenate as described herein. In embodiments relating to polysaccharides, including exopolysaccharides, the composition may comprise a homogenous or a heterogeneous population of polysaccharide molecules, including sulfated polysaccharides as non-limiting embodiments. Non-limiting examples of homogenous populations include those containing a single type of polysaccharide molecule, such as that with the same structure and molecular weight. Non-limiting examples of heterogeneous populations include those containing more than one type of polysaccharide molecule, such as a mixture of polysaccharides having a molecular weight (MW) within a range or a MW above or below a MW value. For example, the Porphyridium sp. exopolysaccharide is typically produced in a range of sizes from 3-5 million Daltons. Of course a polysaccharide containing composition of the invention may be optionally protease treated, or reduced in the amount of protein, as described above.

In some embodiments, a composition of the invention may comprise one or more polysaccharides produced by microalgae that have not been recombinantly modified. The microalgae may be those which are naturally occurring or those which have been maintained in culture in the absence of alteration by recombinant DNA techniques or genetic engineering.

In other embodiments, the polysaccharides are those from modified microalgae, such as, but not limited to, microalgae modified by recombinant techniques. Non-limiting examples of such techniques include introduction and/or expression of an exogenous nucleic acid sequence encoding a gene product; genetic manipulation to decrease or inhibit expression of an endogenous microalgal gene product; and/or genetic manipulation to increase expression of an endogenous microalgal gene product. The invention contemplates recombinant modification of the various microalgae species described herein. In some embodiments, the microalgae is from the genus Porphyridium.

Polysaccharides provided by the invention that are produced by genetically modified microalgae or microalgae that are provided with an exogenous carbon source can be distinct from those produced by microalgae cultured in minimal growth media under photoautotrophic conditions (ie: in the absence of a fixed carbon source) at least in that they contain a different monosaccharide content relative to polysaccharides from unmodified microalgae or microalgae cultured in minimal growth media under photoautotrophic conditions. Non-limiting examples include polysaccharides having an increased amount of arabinose (Ara), rhamnose (Rha), fucose (Fuc), xylose (Xyl), glucuronic acid (GlcA), galacturonic acid (GalA), mannose (Man), galactose (Gal), glucose (Glc), N-acetyl galactosamine (GalNAc), N-acetyl glucosamine (GlcNAc), and/or N-acetyl neuraminic acid (NANA), per unit mass (or per mole) of polysaccharide, relative to polysaccharides from either non-genetically modified microalgae or microalgae cultured photoautotrophically. An increased amount of a monosaccharide may also be expressed in terms of an increase relative to other monosaccharides rather than relative to the unit mass, or mole, of polysaccharide. An example of genetic modification leading to production of modified polysaccharides is transforming a microalgae with a carbohydrate transporter gene, and culturing a transformant in the presence of a monosaccharide which is transported into the cell from the culture media by the carbohydrate transporter protein encoded by the carbohydrate transporter gene. In some instances the culture can be in the dark, where the monosaccharide, such as glucose, is used as the sole energy source for the cell. In other instances the culture is in the light, where the cells undergo photosynthesis and therefore produce monosaccharides such as glucose in the chloroplast and transport the monosaccharides into the cytoplasm, while additional exogenously provided monosaccharides are transported into the cell by the carbohydrate transporter protein. In both instances monosaccharides from the cytoplasm are transported into the endoplasmic reticulum, where polysaccharide synthesis occurs. Novel polysaccharides produced by non-genetically engineered microalgae can also be generated by nutritional manipulation, ie: exogenously providing carbohydrates in the culture media that are taken up through endogenous transport mechanisms. Uptake of the exogenously provided carbohydrates can be induced, for example, by culturing the cells in the dark, thereby forcing the cells to utilize the exogenously provided carbon source. For example, Porphyridium cells cultured in the presence of 7% glycerol in the dark produce a novel polysaccharide because the intracellular carbon flux under these nutritionally manipulated conditions is different from that under photosynthetic conditions. Insertion of carbohydrate transporter genes into microalgae facilitates, but is not strictly necessary for, polysaccharide structure manipulation because expression of such genes can significantly increase the concentration of a particular monosaccharide in the cytoplasm of the cell. Many carbohydrate transporter genes encode proteins that transport more than one monosaccharide, albeit with different affinities for different monosaccharides (see for example Biochimica et Biophysica Acta 1465 (2000) 263-274). In some instances a microalgae species can be transformed with a carbohydrate transporter gene and placed under different nutritional conditions, wherein one set of conditions includes the presence of exogenously provided galactose, and the other set of conditions includes the presence of exogenously provided xylose, and the transgenic species produces structurally distinct polysaccharides under the two conditions. By altering the identity and concentration of monosaccharides in the cytoplasm of the microalgae, through genetic and/or nutritional manipulation, the invention provides novel polysaccharides. Nutritional manipulation can also be performed, for example, by culturing the microalgae in the presence of high amounts of sulfate, as described herein. In some instances nutritional manipulation includes addition of one or more exogenously provided carbon sources as well as one or more other non-carbohydrate culture component, such as 50 mM MgSO₄.

In some embodiments, the increase in one or more of the above listed monosaccharides in a polysaccharide may be from below to above detectable levels and/or by at least about 5%, to at least about 2000%, relative to a polysaccharide produced from the same microalgae in the absence of genetic or nutritional manipulation. Therefore an increase in one or more of the above monosaccharides, or other carbohydrates listed in Tables 2 or 3, by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 105%, at least about 110%, at least about 150%, at least about 200%, at least about 250%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 1000%, at least about 1100%, at least about 1200%, at least about 1300%, at least about 1400%, at least about 1500%, at least about 1600%, at least about 1700%, at least about 1800%, or at least about 1900%, or more, may be used in the practice of the invention.

In cases wherein the polysaccharides from unmodified microalgae do not contain one or more of the above monosaccharides, the presence of the monosaccharide in a microalgal polysaccharide indicates the presence of a polysaccharide distinct from that in unmodified microalgae. Thus using particular strains of Porphyridium sp. and Porphyridium cruentum as non-limiting examples, the invention includes modification of these microalgae to incorporate arabinose and/or fucose, because polysaccharides from two strains of these species do not contain detectable amounts of these monosaccharides (see Example 5 herein). In another non-limiting example, the modification of Porphyridium sp. to produce polysaccharides containing a detectable amount of glucuronic acid, galacturonic acid, or N-acetyl galactosamine, or more than a trace amount of N-acetyl glucosamine, is specifically included in the instant disclosure. In a further non-limiting example, the modification of Porphyridium cruentum to produce polysaccharides containing a detectable amount of rhamnose, mannose, or N-acetyl neuraminic acid, or more than a trace amount of N-acetyl-glucosamine, is also specifically included in the instant disclosure.

Put more generally, the invention includes a method of producing a polysaccharide comprising culturing a microalgae cell in the presence of at least about 0.01 micromolar of an exogenously provided fixed carbon compound, wherein the compound is incorporated into the polysaccharide produced by the cell. In some embodiments, the compound is selected from Table 2 or 3. The cells may optionally be selected from the species listed in Table 1, and cultured by modification, using the methods disclosed herein, or the culture conditions also lusted in Table 1.

The methods may also be considered a method of producing a glycopolymer by culturing a transgenic microalgal cell in the presence of at least one monosaccharide, wherein the monosaccharide is transported by the transporter into the cell and is incorporated into a microalgal polysaccharide.

In some embodiments, the cell is selected from Table 1, such as where the cell is of the genus Porphyridium, as a non-limiting example. In some cases, the cell is selected from Porphyridium sp. and Porphyridium cruentum. Embodiments include those wherein the polysaccharide is enriched for the at least one monosaccharide compared to an endogenous polysaccharide produced by a non-transgenic cell of the same species. The monosaccharide may be selected from Arabinose, Fructose, Galactose, Glucose, Mannose, Xylose, Glucuronic acid, Glucosamine, Galactosamine, Rhamnose and N-acetyl glucosamine.

These methods of the invention are facilitated by use of a transgenic cell expressing a sugar transporter, optionally wherein the transporter has a lower Kₘ for glucose than at least one monosaccharide selected from the group consisting of galactose, xylose, glucuronic acid, mannose, and rhamnose. In other embodiments, the transporter has a lower Kₘ for galactose than at least one monosaccharide selected from the group consisting of glucose, xylose, glucuronic acid, mannose, and rhamnose. In additional embodiments, the transporter has a lower Kₘ for xylose than at least one monosaccharide selected from the group consisting of glucose, galactose, glucuronic acid, mannose, and rhamnose. In further embodiments, the transporter has a lower Kₘ for glucuronic acid than at least one monosaccharide selected from the group consisting of glucose, galactose, xylose, mannose, and rhamnose. In yet additional embodiments, the transporter has a lower Kₘ for mannose than at least one monosaccharide selected from the group consisting of glucose, galactose, xylose, glucuronic acid, and rhamnose. In yet further embodiments, the transporter has a lower Kₘ for rhamnose than at least one monosaccharide selected from the group consisting of glucose, galactose, xylose, glucuronic acid, and mannose. Manipulation of the concentration and identity of monosaccharides provided in the culture media, combined with use of transporters that have a different Kₘ for different monosaccharides, provides novel polysaccharides. These general methods can also be used in cells other than microalgae, for example, bacteria that produce polysaccharides.

In alternative embodiments, the cell is cultured in the presence of at least two monosaccharides, both of which are transporter by the transporter. In some cases, the two monosaccharides are any two selected from glucose, galactose, xylose, glucuronic acid, rhamnose and mannose.

In one non-limiting example, the method comprises providing a transgenic cell containing a recombinant gene encoding a monosaccharide transporter; and culturing the cell in the presence of at least one monosaccharide, wherein the monosaccharide is transported by the transporter into the cell and is incorporated into a polysaccharide of the cell. It is pointed out that transportation of a monosaccharide from the media into a microalgal cell allows for the monosaccharide to be used as an energy source, as disclosed below, and for the monosaccharide to be transported into the endoplasmic reticulum (ER) by cellular transporters. In the ER, polysaccharide production and glycosylation, occurs such that in the presence of exogenously provided monosaccharides, the sugar content of the microalgal polysaccharides change.

In some aspects, the invention includes a novel microalgal polysaccharide, such as from microalgae of the genus Porphyridium, comprising detectable amounts of xylose, glucose, and galactose wherein the molar amount of one or more of these three monosaccharides in the polysaccharide is not present in a polysaccharide of Porphyridium that is not genetically or nutritionally modified. An example of a non-nutritionally and non-genetically modified Porphyridium polysaccharide can be found, for example, in Jones R., Journal of Cellular Comparative Physiology 60; 61-64 (1962). In some embodiments, the amount of glucose, in the polysaccharide, is at least about 65% of the molar amount of galactose in the same polysaccharide. In other embodiments, glucose is at least about 70%, at least about 75%, at least about 80%, at least about 85%, at least about 90%, at least about 95%, at least about 100%, at least about 105%, at least about 110%, at least about 120%, at least about 130%, at least about 140%, at least about 150%, at least about 200%, at least about 250%, at least about 300%, at least about 350%, at least about 400%, at least about 450%, at least about 500%, or more, of the molar amount of galactose in the polysaccharide. Further embodiments of the invention include those wherein the amount of glucose in a microalgal polysaccharide is equal to, or approximately equal to, the amount of galactose (such that the amount of glucose is about 100% of the amount of galactose). Moreover, the invention includes microalgal polysaccharides wherein the amount of glucose is more than the amount of galactose.

Alternatively, the amount of glucose, in the polysaccharide, is less than about 65% of the molar amount of galactose in the same polysaccharide. The invention thus provides for polysaccharides wherein the amount of glucose is less than about 60%, less than about 55%, less than about 50%, less than about 45%, less than about 40%, less than about 35%, less than about 30%, less than about 25%, less than about 20%, less than about 15%, less than about 10%, or less than about 5% of the molar amount of galactose in the polysaccharide.

In other aspects, the invention includes a microalgal polysaccharide, such as from microalgae of the genus Porphyridium, comprising detectable amounts of xylose, glucose, galactose, mannose, and rhamnose, wherein the molar amount of one or more of these five monosaccharides in the polysaccharide is not present in a polysaccharide of non-genetically modified and/or non-nutritionally modified microalgae. In some embodiments, the amount of rhamnose in the polysaccharide is at least about 100% of the molar amount of mannose in the same polysaccharide. In other embodiments, rhamnose is at least about 110%, at least about 120%, at least about 130%, at least about 140%, at least about 150%, at least about 200%, at least about 250%, at least about 300%, at least about 350%, at least about 400%, at least about 450%, or at least about 500%, or more, of the molar amount of mannose in the polysaccharide. Further embodiments of the invention include those wherein the amount of rhamnose in a microalgal polysaccharide is more than the amount of mannose on a molar basis.

Alternatively, the amount of rhamnose, in the polysaccharide, is less than about 75% of the molar amount of mannose in the same polysaccharide. The invention thus provides for polysaccharides wherein the amount of rhamnose is less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5% of the molar amount of mannose in the polysaccharide.

In additional aspects, the invention includes a microalgal polysaccharide, such as from microalgae of the genus Porphyridium, comprising detectable amounts of xylose, glucose, galactose, mannose, and rhamnose, wherein the amount of mannose, in the polysaccharide, is at least about 130% of the molar amount of rhamnose in the same polysaccharide. In other embodiments, mannose is at least about 140%, at least about 150%, at least about 200%, at least about 250%, at least about 300%, at least about 350%, at least about 400%, at least about 450%, or at least about 500%, or more, of the molar amount of rhamnose in the polysaccharide.

Alternatively, the amount of mannose, in the polysaccharide, is equal to or less than the molar amount of rhamnose in the same polysaccharide. The invention thus provides for polysaccharides wherein the amount of mannose is less than about 95%, less than about 90%, less than about 85%, less than about 80%, less than about 75%, less than about 70%, less than about 65%, less than about 60%, less than about 60%, less than about 55%, less than about 50%, less than about 45%, less than about 40%, less than about 35%, less than about 30%, less than about 25%, less than about 20%, less than about 15%, less than about 10%, or less than about 5% of the molar amount of rhamnose in the polysaccharide.

In further aspects, the invention includes a microalgal polysaccharide, such as from microalgae of the genus Porphyridium, comprising detectable amounts of xylose, glucose, and galactose, wherein the amount of galactose in the polysaccharide, is at least about 100% of the molar amount of xylose in the same polysaccharide. In other embodiments, rhamnose is at least about 105%, at least about 110%, at least about 120%, at least about 130%, at least about 140%, at least about 150%, at least about 200%, at least about 250%, at least about 300%, at least about 350%, at least about 400%, at least about 450%, or at least about 500%, or more, of the molar amount of mannose in the polysaccharide. Further embodiments of the invention include those wherein the amount of galactose in a microalgal polysaccharide is more than the amount of xylose on a molar basis.

Alternatively, the amount of galactose, in the polysaccharide, is less than about 55% of the molar amount of xylose in the same polysaccharide. The invention thus provides for polysaccharides wherein the amount of galactose is less than about 50%, less than about 45%, less than about 40%, less than about 35%, less than about 30%, less than about 25%, less than about 20%, less than about 15%, less than about 10%, or less than about 5% of the molar amount of xylose in the polysaccharide.

In yet additional aspects, the invention includes a microalgal polysaccharide, such as from microalgae of the genus Porphyridium, comprising detectable amounts of xylose, glucose, glucuronic acid and galactose, wherein the molar amount of one or more of these five monosaccharides in the polysaccharide is not present in a polysaccharide of unmodified microalgae. In some embodiments, the amount of glucuronic acid, in the polysaccharide, is at least about 50% of the molar amount of glucose in the same polysaccharide. In other embodiments, glucuronic acid is at least about 55%, at least about 60%, at least about 65%, at least about 70%, at least about 75%, at least about 80%, at least about 85%, at least about 90%, at least about 95%, at least about 100%, at least about 105%, at least about 110%, at least about 120%, at least about 130%, at least about 140%, at least about 150%, at least about 200%, at least about 250%, at least about 300%, at least about 350%, at least about 400%, at least about 450%, or at least about 500%, or more, of the molar amount of glucose in the polysaccharide. Further embodiments of the invention include those wherein the amount of glucuronic acid in a microalgal polysaccharide is more than the amount of glucose on a molar basis.

In other embodiments, the exopolysaccharide, or cell homogenate polysaccharide, comprises glucose and galactose wherein the molar amount of glucose in the exopolysaccharide, or cell homogenate polysaccharide, is at least about 55% of the molar amount of galactose in the exopolysaccharide or polysaccharide. Alternatively, the molar amount of glucose in the exopolysaccharide, or cell homogenate polysaccharide, is at least about 60%, at least about 65%, at least about 70%, at least about 75%, at least about 80%, at least about 85%, at least about 90%, or at least about 100% of the molar amount of galactose in the exopolysaccharide or polysaccharide.

In yet further aspects, the invention includes a microalgal polysaccharide, such as from microalgae of the genus Porphyridium, comprising detectable amounts of xylose, glucose, glucuronic acid, galactose, at least one monosaccharide selected from arabinose, fucose, N-acetyl galactosamine, and N-acetyl neuraminic acid, or any combination of two or more of these four monosaccharides.

IV Topical and Mucosal Application of Polysaccharides

A. General

Compositions, comprising polysaccharides, whole cell extracts, or mixtures of polysaccharides and whole cell extracts, are provided for topical application or non-systemic administration. The polysaccharide may be any one or more of the microalgal polysaccharides disclosed herein, including those produced by a species, or a combination of two or more species, in Table 1. Similarly, a whole cell extract may be that prepared from a microalgal species, or a combination of two or more species, in Table 1. In some embodiments, polysaccharides, such as exopolysaccharides, and cell extracts from microalgae of the genus Porphyridium are used in the practice of the invention. A composition of the invention may comprise from between about 0.001% and about 100%, about 0.01% and about 90%, about 0.1% and about 80%, about 1% and about 70%, about 2% and about 60%, about 4% and about 50%, about 6% and about 40%, about 7% and about 30%, about 8% and about 20%, or about 10% polysaccharide, cell extract, by weight.

Topical compositions are usually formulated with a carrier, such as in an ointment or a cream, and may optionally include a fragrance. One non-limiting class of topical compositions is that of cosmeceuticals. Other non-limiting examples of topical formulations include gels, solutions, impregnated bandages, liposomes, or biodegradable microcapsules as well as lotions, sprays, aerosols, suspensions, dusting powder, impregnated bandages and dressings, biodegradable polymers, and artificial skin. Another non-limiting example of a topical formulation is that of an ophthalmic preparation. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water.

In some embodiments, the polysaccharides contain fucose moieties. In other embodiments, the polysaccharides are sulfated, such as exopolysaccharides from microalgae of the genus Porphyridium. In some embodiments, the polysaccharides will be those from a Porphyridium species, such as one that has been subject to genetic and/or nutritional manipulation to produce polysaccharides with altered monosaccharide content and/or altered sulfation.

In additional embodiments, a composition of the invention comprises a microalgal cell homogenate and a topical carrier. In some embodiments, the homogenate may be that of a species listed in Table 1 or may be material produced by a species in the table.

In further embodiments, a composition comprising purified microalgal polysaccharide and a carrier suitable for topical administration also contains a fusion (or chimeric) protein associated with the polysaccharide. In some embodiments, the fusion protein comprises a first protein, or polypeptide region, with at least about 60% amino acid identity with the protein of SEQ ID NO: 21. In other embodiments, the first protein has at least about 70%, at least about 75%, at least about 80%, at least about 85%, at least about 90%, at least about 95%, or at least about 98%, or higher, amino acid identity with the sequence of SEQ ID NO:21.

The fusion protein may comprise a second protein, or polypeptide region, with a homogenous or heterologous sequence. A non-limiting example of the second protein is an antibody. Non-limiting examples of antibodies for use in this aspect of the invention include an antibody that selectively binds to an antigen from a pathogen selected from HIV, Herpes Simplex Virus, gonorrhea, Chlamydia, Human Papillomavirus, and Trichomoniasis. In some embodiments, the antibody is a humanized antibody. Examples of antibodies that specifically bind to antigens on infectious disease pathogens are: (Expert Opin Biol Ther. 2004 March; 4(3):387-96; Expert Opin Biol Ther. 2005 October; 5(10):1359-72.; Nat Rev Microbiol. 2004 September; 2(9):695-703.; Trends Microbiol. 2004 June; 12(6):259-63; Emerg Infect Dis. 2002 August; 8(8):833-41; Infect Immun. 2002 February; 70(2):544-60; Nat Biotechnol. 2002 June; 20(6):597-601; J Infect Dis. 2005 Feb. 15; 191(4):507-14; Proc Natl Acad Sci USA. 2004 Feb. 24; 101(8):2536-41; Mol Immunol. 2005 January; 42(1):125-36; J Virol Methods. 2004 Sep. 1; 120(1):87-96; J Virol. 1997 October; 71(10):7198-206; J Virol. 1998 December; 72(12):9788-94; J Virol. 1999 May; 73(5):4009-18; US Patent App. 20040058403). Another example of an antibody capable of neutralizing an infectious disease is the 80R antibody (J Virol. 2005 May; 79(10):5900-6; Proc Natl Acad Sci USA. 2004 Feb. 24; 101(8):2536-41), which neutralizes the SARS virus and can be expressed in microalgae along with other SARS-neutralizing antibodies (BMC Infect Dis. 2005 Sep 19; 5:73; Antivir Ther. 2005; 10(5):681-90; J Biomed Sci. 2005; 12(5):711-27).

B. Methods of Formulation

Polysaccharide compositions for topical application can be formulated by first preparing a purified preparation of polysaccharide. As a non-limiting example, the polysaccharide from aqueous growth media is precipitated with an alcohol, resuspended in a dilute buffer, and mixed with a carrier suitable for application to human skin or mucosal tissue, including the vaginal canal. Alternatively, the polysaccharide can be purified from growth media and concentrated by tangential flow filtration or other filtration methods, and formulated as described above. Intracellular polysaccharides can be also formulated in a similar or identical manner after purification from other cellular components.

As a non-limiting example, the invention includes a method of formulating a cosmeceutical composition, said method comprising culturing microalgal cells in suspension under conditions to allow cell division; separating the microalgal cells from culture media, wherein the culture media contains exopolysaccharide molecules produced by the microalgal cells; separating the exopolysaccharide molecules from other molecules present in the culture media; homogenizing the microalgal cells; and adding the separated exopolysaccharide molecules to the cells before, during, or after homogenization. In some embodiments, the microalgal cells are from the genus Porphyridium.

Examples of polysaccharides, both secreted and intracellular, that are suitable for formulation with a carrier for topical application are listed in Table I.

Examples of carriers suitable for formulating polysaccharide are described above. Ratios of homogenate:carrier are typically in the range of about 0.001:1 to about 1:1 (volume:volume), although the invention comprises ratios outside of this range, such as, but not limited to, about 0.01:1 and about 0.1:1. In the method of mucosal application, the polysaccharide compound is administered to the mucosa of the subject. Thus, specific examples of the mucosal administration include nasal, oral, rectal and vaginal. Nasal administration can be by nasal aerosol spray or nebulizer among other well practiced methods. Rectal and vaginal administration can be by a variety of: methods, including lavage (douches, enemas, etc.), suppositories, creams, gels, etc. For nasal administration, an aerosol spray or nebulizer can be used.

C. Methods of Screening Compounds for Antiviral Activity

Compounds including the novel polysaccharides of the invention can be screened for the ability to neutralize viruses. Viral neutralization assays are known in the art. See for example Akanitapichat P et al., J Ethnopharmacol. 2005 Nov. 15 (PMID: 16298095); J Biomed Sci. 2005 December; 12(6):1021-34; J Biol Chem. 2005 Sep. 16; 280(37):32193-9; Phytother Res. 2004 July; 18(7):551-5; Mem Inst Oswaldo Cruz. 2003 September; 98(6):843-8; Antiviral Res. 2003 August; 59(3):143-54; Antivir Chem Chemother. 2005; 16(5):303-13; Clin Exp Immunol. 2005 November; 142(2):327-32; J Med Virol. 2001 December; 65(4):649-58.

V Compositions for Non-Systemic Administration of Polysaccharides

A. General

Compositions for non-systemic administration include those formulated for localized administration with little or slow release to other parts of a treated subject's body. Non-limiting examples of non-systemic administration include intravaginal application such as via a suppository, cream or foam; and rectal administration via suppository, irrigation or other suitable means. In some embodiments, the composition is formulated for the treatment of sexually transmitted diseases, such as those caused by viral agents.

Polysaccharides from microalgae provided herein posses potent antiviral activity (see references cited in Table 1). In additional embodiments, polysaccharides with lubricant properties (see for example Porphyridium polysaccharides) are used in the practice of certain aspects of the invention. These polysaccharides may be formulated in solutions that are added to prophylactic devices. Moreover, the polysaccharides may be one or more described herein, optionally sulfated. In many embodiments, the polysaccharide is produced by a microalgal species, or two or more species, listed in Table 1. In some embodiments, the microalgae is Porphyridium sp. or Porphyridium cruentum.

Thus, the invention includes a sexually transmitted disease prevention composition, said composition comprising 1) a solution comprising a polysaccharide produced from microalgae; and 2) a prophylactic device. In some embodiments, the solution and device are kept separate, but packaged together as a single unit for sale. The solution may be applied to the device by the end user before actual use. Alternatively the solution and device are packaged so that the solution is in direct contact with the device. The prophylactic devices include, but are not limited to, condoms, sponges, and diaphragms.

In some embodiments, the devices are packaged with a lubricant. In other embodiments, the polysaccharide acts as a lubricant and so no other lubricant is needed. In such embodiments, the substance in the composition providing a lubricant function and the substance in the composition providing antiviral activity are the same substance. Alternatively, a combination of a lubricant, such as a cream or lotion, with the polysaccharide of the invention may be used.

In some embodiments, the polysaccharide is in a composition with a carrier used with a prophylactic device described above. Non-limiting examples of a carrier include a spermicide and a lubricant. In other embodiments of the invention, a triple composition, comprising spermicide, lubricant and the polysaccharide, may be used.

In further embodiments, the polysaccharide is associated with a fusion (or chimeric) protein comprising a first protein (or polypeptide region) with at least about 60% amino acid identity with the protein of SEQ ID NO: 21. In some cases, the first protein has at least about 70%, at least about 75%, at least about 80%, at least about 85%, at least about 90%, at least about 95%, or at least about 98%, or higher, amino acid identity with the sequence of SEQ ID NO:21.

The fusion protein may comprise a second protein, or polypeptide region, with a homogenous or heterologous sequence. One non-limiting example of the second protein is an antibody. In some embodiments, the antibody is selective for binding to an antigen of a pathogen, or opportunistic organism, involved in a sexually transmitted disease. Non-limiting examples of antibodies include those that bind an antigen from a pathogen selected from HIV, Herpes Simplex Virus, gonorrhea, Chlamydia, Human Papilloma Virus, and Trichomoniasis.

B. Methods of Use

The polysaccharides of the invention may be used in the same or a similar manner. In some embodiments, the polysaccharides will be those from a Porphyridium species, such as one that has been subject to genetic and/or nutritional manipulation to produce polysaccharides with altered monosaccharide content.

For systemic administration, polysaccharides can be fragmented to reuce viscosity using such methods as sonication. For example, the Porphyridium polysaccharide can be fragmented from its naturally occurring molecular weight of about 4.5 million Daltons to an average molecular weight of about 100,000 daltons. As the average molecular weight of the polysaccharide is reduced, the viscosity is also reduced. Polysaccharide preparations from Porphyridium can be fragmented to an average molecular weight of 50,000, 100,000, 200,000, 300,000 and 400,000 daltons for example. A preferred composition for parenteral administration is an exopolysaccharide preparation produced from the culture media of cells of the genus Porphyridium, wherein the polysaccharide has an average molecular weight of less than 300,000, the preparation is substantially free of protein, and the preparation is sterile.

For systemic administration, polysaccharides can be formulated with carriers, excipients, and other compounds. pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d.alpha-tocopherol polyethyleneglycol 1000 succinate, or other similar polymeric delivery matrices or systems, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-beta-cyclodextrins, or other solublized derivatives may also be advantageously used to enhance delivery of therapeutically-effective plant essential oil compounds of the present invention.

The polysaccharide compositions of this invention may be administered orally, parenterally, by inhalation spray, topically, rectally, nasally, buccally, vaginally or via an implanted reservoir, however, oral administration or administration by injection is preferred. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intrasynovial, intrasternal, intrathecal, intralesional and intracranial injection or infusion techniques.

The polysaccharide compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringers solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as Ph. Helv or a similar alcohol.

Sterile injectable polysaccharide compositions preferably contain less than 1% protein as a function of dry weight of the composition, more preferably less than 0.1% protein, more preferably less than 0.01% protein, less than 0.001% protein, less than 0.0001% protein, more preferably less than 0.00001% protein, more preferably less than 0.000001% protein.

The polysaccharide compositions of the present invention may be orally administered in any orally acceptable dosage form including, but not limited to, capsules, tablets, and aqueous suspensions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added.

The polysaccharide compositions of the present invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

VI Gene Expression in Microalgae

Genes can be expressed in microalgae by providing, for example, coding sequences in operable linkage with promoters.

An exemplary vector design for expression of a gene in microalgae contains a first gene in operable linkage with a promoter active in algae, the first gene encoding a protein that imparts resistance to an antibiotic or herbicide. Optionally the first gene is followed by a 3′ untranslated sequence containing a polyadenylation signal. The vector may also contain a second promoter active in algae in operable linkage with a second gene. The second gene can encode any protein, for example an enzyme that produces small molecules or a mammalian growth hormone that can be advantageously present in a nutraceutical.

It is preferable to use codon-optimized cDNAs: for methods of recoding genes for expression in microalgae, see for example US patent application 20040209256.

It has been shown that many promoters in expression vectors are active in algae, including both promoters that are endogenous to the algae being transformed algae as well as promoters that are not endogenous to the algae being transformed (ie: promoters from other algae, promoters from plants, and promoters from plant viruses or algae viruses). Example of methods for transforming microalgae, in addition to those demonstrated in the Examples section below, including methods comprising the use of exogenous and/or endogenous promoters that are active in microalgae, and antibiotic resistance genes functional in microalgae, have been described. See for example; Curr Microbiol. 1997 December; 35(6):356-62 (Chlorella vulgaris); Mar Biotechnol (NY). 2002 January; 4(1):63-73 (Chlorella ellipsoidea); Mol Gen Genet. 1996 Oct. 16; 252(5):572-9 (Phaeodactylum tricornutum); Plant Mol Biol. 1996 April; 31(1):1-12 (Volvox carteri); Proc Natl Acad Sci USA. 1994 Nov. 22; 91(24):11562-6 (Volvox carteri); Falciatore A, Casotti R, Leblanc C, Abrescia C, Bowler C, PMID: 10383998, 1999 May; 1(3):239-251 (Laboratory of Molecular Plant Biology, Stazione Zoologica, Villa Comunale, I-80121 Naples, Italy) (Phaeodactylum tricornutum and Thalassiosira weissflogii); Plant Physiol. 2002 May; 129(1):7-12. (Porphyridium sp.); Proc Natl Acad Sci USA. 2003 Jan. 21; 100(2):438-42. (Chlamydomonas reinhardtii); Proc Natl Acad Sci USA. 1990 February; 87(3):1228-32. (Chlamydomonas reinhardtii); Nucleic Acids Res. 1992 Jun. 25; 20(12):2959-65; Mar Biotechnol (NY). 2002 January; 4(1):63-73 (Chlorella); Biochem Mol Biol Int. 1995 August; 36(5):1025-35 (Chlamydomonas reinhardtii); J Microbiol. 2005 August; 43(4):361-5 (Dunaliella); Yi Chuan Xue Bao. 2005 April; 32(4):424-33 (Dunaliella); Mar Biotechnol (NY). 1999 May; 1(3):239-251. (Thalassiosira and Phaedactylum); Koksharova, Appl Microbiol Biotechnol 2002 February; 58(2):123-37 (various species); Mol Genet Genomics. 2004 February; 271(1):50-9 (Thermosynechococcus elongates); J. Bacteriol. (2000), 182, 211-215; FEMS Microbiol Lett. 2003 Apr. 25; 221(2):155-9; Plant Physiol. 1994 June; 105(2):635-41; Plant Mol Biol. 1995 December; 29(5):897-907 (Synechococcus PCC 7942); Mar Pollut Bull. 2002; 45(1-12):163-7 (Anabaena PCC 7120); Proc Natl Acad Sci USA. 1984 March; 81(5):1561-5 (Anabaena (various strains)); Proc Natl Acad Sci USA. 2001 Mar. 27; 98(7):4243-8 (Synechocystis); Wirth, Mol Gen Genet 1989 March; 216(1):175-7 (various species); Mol Microbiol, 2002 June; 44(6):1517-31 and Plasmid, 1993 September; 30(2):90-105 (Fremyella diplosiphon); Hall et al. (1993) Gene 124: 75-81 (Chlamydomonas reinhardtii); Gruber et al. (1991). Current Micro. 22: 15-20; Jarvis et al. (1991) Current Genet. 19: 317-322 (Chlorella); for additional promoters see also Table 1 from U.S. Pat. No. 6,027,900).

Suitable promoters may be used to express a nucleic acid sequence in microalgae. In some embodiments, the sequence is that of an exogenous gene or nucleic acid. In particular embodiments, the exogenous gene is one that encodes a carbohydrate transporter protein. Such a gene may be advantageously expressed in a microalgal cell to allow entry of a monosaccharide transported by the transporter protein. In other embodiments, the exogenous gene can encode a fusion of a polysaccharide-associated protein and an antibody. In cases of an exogenous nucleic acid coding sequence, the codon usage may be optionally optimized in whole or in part to facilitate expression in microalgae.

The invention thus includes, in some embodiments, a microalgal cell comprising an exogenous gene that encodes a carbohydrate transporter protein. The cell may be that of the genus Porphyridum as a non-limiting example. Non-limiting examples of genes encoding carbohydrate transporters to facilitate the uptake of exogenously provided carbohydrates include SEQ ID NOs: 13, 15, 17, 19 and 20 as provided herein. In some embodiments the nucleic acid sequence encodes a protein with at least about 60% amino acid sequence identity with a protein with a sequence represented by one of SEQ ID NOs: 13, 15, 17, 19 and 20. In other embodiments, the nucleic acid sequence encodes a protein with at least about 70%, at least about 75%, at least about 80%, at least about 85%, at least about 90%, at least about 95%, or at least about 98%, or higher, amino acid identity with a sequence of these SEQ ID NOs: 13, 15, 17, 19 and 20. In further embodiments, the nucleic acid sequence has at least 60% nucleotide identity with a nucleic acid molecule with a sequence represented by one of SEQ ID NOs: 14, 16 and 18. In other embodiments, the nucleic acid sequence has at least about 70%, at least about 75%, at least about 80%, at least about 85%, at least about 90%, at least about 95%, or at least about 98%, or higher, nucleic acid identity with a sequence of these SEQ ID NOs.

In other embodiments, the invention provides for the expression of a protein sequence found to be tightly associated with microalgal polysaccharides. One non-limiting example is the protein of SEQ ID NO: 21, which has been shown to be tightly associated with, but not covalently bound to, the polysaccharide from Porphyridium sp. (see J. Phycol. 40: 568-580 (2004)). When Porphyridium culture media is subjected to tangential flow filtration using a filter containing a pore size well in excess of the molecular weight of the protein of SEQ ID NO: 21, the polysaccharide in the retentate contains detectable amounts of the protein, indicating its tight association with the polysaccharide. The calculated molecular weight of the protein is approximately 58 kD, however with glycosylation the protein is approximately 66 kD.

Such a protein may be expressed directly such that it will be present with the polysaccharides of the invention or expressed as part of a fusion or chimeric protein as described herein. As a fusion protein, the portion that is tightly associated with a microalgal polysaccharide effectively links the other portion(s) to the polysaccharide. A fusion protein may comprise a second protein or polypeptide, with a homogenous or heterologous sequence. A homogenous sequence would result in a dimer or multimer of the protein while a heterologous sequence can introduce a new functionality, including that of a bioactive protein or polypeptide.

A fusion between the polysaccharide binding protein and antibodies that specifically bind to and neutralize a pathogen are included in the invention. Non-limiting examples include anti-HIV antibodies, like the 2G12 antibody (see Proc Natl Acad Sci USA. 2005 Sep. 20; 102(38):13372-7); the 1RHH_B antibody (see Clin Exp Immunol. 2005 July; 141(1):72-80); the scFv102 antibody (see J Gen Virol. 2005 June; 86(Pt 6):1791-800); and the microAb antibody (see Nat Med. 2005 June; 11(6):615-22; 2G12, 2F5, 4E10, 2g12 Fab 1ZLS_L). These and other antibodies, preferably antibodies that specifically bind to infectious disease agents, can also be expressed in algae without being fused to any other proteins. The biomass containing the recombinant antibodies can be administered orally to deliver the antibodies to a mammal for prophylaxis or treatment.

One advantage to a fusion is that the bioactivity of the polysaccharide and the bioactivity from the protein can be combined in a product without increasing the manufacturing cost over only purifying the exopolysaccharide. As a non-limiting example, the potent antiviral properties of a Porphyridium polysaccharide can be combined with the potent antiviral properties of an antiviral antibody in a fusion, however the polysaccharide:antibody combination can be isolated to a high level of purity using tangential flow filtration.

In other embodiments, the invention includes genetic expression methods comprising the use of an expression vector. In one method, a microalgal cell, such as a Porphyridium cell, is transformed with a dual expression vector under conditions wherein vector mediated gene expression occurs. The expression vector may comprise a resistance cassette comprising a gene encoding a protein that confers resistance to an antibiotic, such as zeocin, or another selectable marker such as a carbohydrate transporter gene for selection in the dark in the presence of a fixed carbon source, operably linked to a promoter active in microalgae. The vector may also comprise a second expression cassette comprising a second protein to a promoter active in microalgae. The two cassettes are physically linked in the vector. The transformed cells may be optionally selected based upon the ability to grow in the presence of the antibiotic or other selectable marker under conditions wherein cells lacking the resistance cassette would not grow, such as in the dark. The resistance cassette, as well as the expression cassette, may be taken in whole or in part from another vector molecule.

In one non-limiting example, a method of expressing an exogenous gene in a cell of the genus Porphyridium is provided. The method may comprise operably linking a gene encoding a protein that confers resistance to the antibiotic zeocin to a promoter active in microalgae to form a resistance cassette; operably linking a gene encoding a second protein to a promoter active in microalgae to form a second expression cassette, wherein the resistance cassette and second expression cassette are physically connected to form a dual expression vector; transforming the cell with the dual expression vector; and selecting for the ability to survive in the presence of at least 2.5 ug/ml zeocin, preferably at least 3.0 ug/ml zeocin, and more preferably at least 3.5 ug/ml zeocin, more preferably at least 5.0 ug/ml zeocin.

For sequence comparison to determine percent nucleotide or amino acid identity, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

Another example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra.). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. For identifying whether a nucleic acid or polypeptide is within the scope of the invention, the default parameters of the BLAST programs are suitable. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix. The TBLATN program (using protein sequence for nucleotide sequence) uses as defaults a word length (W) of 3, an expectation (E) of 10, and a BLOSUM 62 scoring matrix. (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

VII Methods of Trophic Conversion

As explained herein, microalgae generally have the ability to live off a fixed carbon sources such as glucose, but many do not have transporters that allow for uptake of the fixed carbon source from the culture media. Microalgae cells can be transformed with a gene that encodes a plasma membrane sugar transporter that allows for the selection of growth in the dark, in the absence of photosynthesis, in the presence of the transporter's substrate sugar. Such transformed cells provide a significant benefit in that the need for light energy is reduced or eliminated because the cells may grow and produce cellular products, including polysaccharides, in the presence of fixed carbon material as the energy source. See for example, Science. 2001 Jun. 15; 292(5524):2073-5. Such growth achieves much higher cell densities in a shorter period of time than photoautotrophic growth.

The transformed microalgal cell may be one that is described above as expressing a sugar transporter. Nucleic acids and vectors for such expression are also described above. For example, nucleic acids encoding carbohydrate transporters such as SEQ ID NOs: 13, 15, 17, 19, 20, and 22-32 are placed in operable linkage with a promoter active in microalgae. Preferably, the nucleic acid encoding a carbohydrate transporter contains preferred codons of the organism the vector is transformed into. For example, the nucleic acids of SEQ ID NOs: 14, 16, and 18 encode the carbohydrate transporter proteins of SEQ ID NOs: 13, 15, and 17, respectively. As a nonlimiting example, a codon-optimized cDNA encoding a carbohydrate transporter protein, optimized for expression in Porphyridium sp., is placed in operable linkage with a promoter and 3′UTR active in microalgae. The vector is used to transform a cell of the genus Porphyridium using methods disclosed herein, including biolistic transformation, electroporation, and glass bead transformation. A preferred promoter is active in more than one species of microalgae, such as for example the Chlamydomonas reinhardtii RBCS2 promoter (SEQ ID NO:34). Any promoter active in microalgae can be used to express a gene in such constructs, and preferred promoters such as RBCS2 and viral promoters have been shown to be active in multiple species of microalgae (see for example Plant Cell Rep. 2005 March; 23(10-11):727-35; J Microbiol. 2005 August; 43(4):361-5; Mar Biotechnol (NY). 2002 January; 4(1):63-73). Promoters, cDNAs, and 3′UTRs, as well as other elements of the vectors, can be generated through cloning techniques using fragments isolated from native sources (see for example Molecular Cloning: A Laboratory Manual, Sambrook et al. (3d edition, 2001, Cold Spring Harbor Press; and U.S. Pat. No. 4,683,202). Alternatively, elements can be generated synthetically using known methods (see for example Gene. 1995 Oct. 16; 164(1):49-53).

Alternatively, cells may be mutagenized and then selected for the ability to grow in the absence of light energy but in the presence of a fixed carbon source.

Thus the invention includes a method of producing microalgal cells that have gained the ability to grow via a fixed carbon source in the absence of photosynthesis. This may also be referred to as trophic conversion of a microalgal cell to no longer be an obligate photoautotroph. In some embodiments, the method comprises identifying or selecting cells that have gained the ability to utilize energy from a fixed carbon source.

In some embodiments, the methods comprise selecting microalgal cells, such as a Porphyridium cell, for the ability to undergo cell division in the absence of light, or light energy. The cells, such as one from a species listed in Table 1, may be those which have been transformed with a sugar transporter or those which have been mutagenized, chemically or non-chemically. The selection may be, for example, on about 0.1% or about 1% glucose, or another fixed carbon source, in the dark. Preferred fixed carbon compounds are listed in Tables 2 and 3.

Non-limiting examples of carbohydrate transporter proteins, optionally operably linked to promoters active in microalgae, as well as expression cassettes and vectors comprising them, have been described above. Alternatively, the nucleic acids may be incorporated into the genome of a microalgal cell such that an endogenous promoter is used to express the transporter. Additional embodiments of the methods include expression of transporters of a carbohydrate selected from Table 2 or 3. Non-limiting examples of mutagenesis include contact or propagation in the presence of a mutagen, such as ultraviolet light, nitrosoguanidine, and/or ethane methyl sulfonate (EMS).

As one non-limiting example, a method of the invention comprises providing a nucleic acid encoding a carbohydrate transporter protein; transforming a Porphyridium cell with the nucleic acid; and selecting for the ability to undergo cell division in the absence of light or in the presence of a carbohydrate that is transported by the carbohydrate transporter protein. In another non-limiting example, a method comprises subjecting a microalgal cell to a mutagen; placing the cell in the presence of a molecule listed in Tables 2 or 3; and selecting for the ability to undergo cell division in the absence of light.

The methods may also be considered to be for trophically converting a microalgal cell to no longer be an obligate phototroph. It is pointed out that the ability to select for loss of obligate phototrophism also provides an alternative means to select for expression of a sugar transporter in the absence of a selectable marker because correct expression and functionality of the transporter is the selectable phenotype when cells are grown in the absence of light for photosynthesis.

It should be apparent to one skilled in the art that various embodiments and modifications may be made to the invention disclosed in this application without departing from the scope and spirit of the invention. All publications mentioned herein are cited for the purpose of describing and disclosing reagents, methodologies and concepts that may be used in connection with the present invention. Nothing herein is to be construed as an admission that these references are prior art in relation to the inventions described herein.

### EXAMPLES

### Example 1

### Growth of Porphyridium cruentum and Porphyridium sp.

Porphyridium sp. (strain UTEX 637) and Porphyridium cruentum (strain UTEX 161) were inoculated into autoclaved 2 liter Erlenmeyer flasks containing an artificial seawater media:

1495 ASW medium recipe from the American Type Culture Collection (components are per 1 liter of media)

| | | |
|---|---|---|
| NaCl | 27.0 | g |
| MgSO₄•7H₂O | 6.6 | g |
| MgCl₂•6H₂O | 5.6 | g |
| CaCl₂•2H₂O | 1.5 | g |
| KNO₃ | 1.0 | g |
| KH₂PO₄ | 0.07 | g |
| NaHCO₃ | 0.04 | g |
| 1.0 M Tris-HCl buffer, pH 7.6 | 20.0 | ml |
| Trace Metal Solution (see below) | 1.0 | ml |
| Chelated Iron Solution (see below) | 1.0 | ml |
| Distilled water | bring to 1.0 | L |

Trace Metal Solution:

| | | |
|---|---|---|
| ZnCl₂ | 4.0 | mg |
| H₃BO₃ | 60.0 | mg |
| CoCl₂•6H₂O | 1.5 | mg |
| CuCl2•2H₂O | 4.0 | mg |
| MnCl₂•4H₂O | 40.0 | mg |
| (NH₄)₆Mo₇O₂₄•4H₂O | 37.0 | mg |
| Distilled water | 100.0 | ml |

Chelated Iron Solution:

| | |
|---|---|
| FeCl₃•4H₂O | 240.0 mg |
| 0.05 M EDTA, pH 7.6 | 100.0 ml |

Media was autoclaved for at least 15 minutes at 121° C.

Inoculated cultures in 2 liter flasks were maintained at room temperature on stir plates. Stir bars were placed in the flasks before autoclaving. A mixture of 5% CO₂ and air was bubbled into the flasks. Gas was filter sterilized before entry. The flasks were under 24 hour illumination from above by standard fluorescent lights (approximately 150 uE/m⁻¹/s⁻¹). Cells were grown for approximately 12 days, at which point the cultures contained approximately of 4×10⁶ cells/mL.

### Example 2

Dense Porphyridium sp. and Porphyridium cruentum cultures were centrifuged at 4000 rcf. The supernatant was subjected to tangential flow filtration in a Millipore Pellicon 2 device through a 1000 kD regenerated cellulose membrane (filter catalog number P2C01MC01). Approximately 4.1 liters of Porphyridium cruentum and 15 liters of Porphyridium sp. supernatants were concentrated to a volume of approximately 200 ml in separate experiments. The concentrated exopolysaccharide solutions were then diafiltered with 10 liters of 1 mM Tris (pH 7.5). The retentate was then flushed with 1 mM Tris (pH 7.5), and the total recovered polysaccharide was lyophilized to completion. Yield calculations were performed by the dimethylmethylene blue (DMMB) assay. The lyophilized polysaccharide was resuspended in deionized water and protein was measured by the bicinchoninic acid (BCA) method. Total dry product measured after lyophilization was 3.28 g for Porphyridium sp. and 2.0 g for Porphyridium cruentum. Total protein calculated as a percentage of total dry product was 12.6% for Porphyridium sp. and 15.0% for Porphyridium cruentum.

### Example 3

Porphyridium sp. culture was centrifuged at 4000 rcf and supernatant was collected. The supernatant was divided into six 30 ml aliquots. Three aliquots were autoclaved for 15 min at 121° C. After cooling to room temperature, one aliquot was mixed with methanol (58.3% vol/vol), one was mixed with ethanol (47.5% vol/vol) and one was mixed with isopropanol (50% vol/vol). The same concentrations of these alcohols were added to the three supernatant aliquots that were not autoclaved. Polysaccharide precipitates from all six samples were collected immediately by centrifugation at 4000 rcf at 20° C. for 10 min and pellets were washed in 20% of their respective alcohols. Pellets were then dried by lyophilization and resuspended in 15 ml deionized water by placement in a 60° C. water bath. Polysaccharide pellets from non-autoclaved samples were partially soluble or insoluble. Polysaccharide pellets from autoclaved ethanol and methanol precipitation were partially soluble. The polysaccharide pellet obtained from isopropanol precipitation of the autoclaved supernatant was completely soluble in water.

### Example 4

Approximately 10 milligrams of purified polysaccharide from Porphyridium sp. and Porphyridium cruentum (described in Example 3) were subjected to monosaccharide analysis.

Monosaccharide analysis was performed by combined gas chromatography/mass spectrometry (GC/MS) of the per-O-trimethylsilyl (TMS) derivatives of the monosaccharide methyl glycosides produced from the sample by acidic methanolysis.

Methyl glycosides prepared from 500 μg of the dry sample provided by the client by methanolysis in 1 M HCl in methanol at 80° C. (18-22 hours), followed by re-N-acetylation with pyridine and acetic anhydride in methanol (for detection of amino sugars). The samples were then per-O-trimethylsilylated by treatment with Tri-Sil (Pierce) at 80° C. (30 mins). These procedures were carried out as previously described described in Merkle and Poppe (1994) Methods Enzymol. 230: 1-15; York, et al. (1985) Methods Enzymol. 118:3-40. GC/MS analysis of the TMS methyl glycosides was performed on an HP 5890 GC interfaced to a 5970 MSD, using a Supelco DB-1 fused silica capillary column (30 m 0.25 mm ID).

Monosaccharide Compositions were Determined as Follows:

TABLE 10
Porphyridium sp. monosaccharide analysis

| Glycosyl residue | Mass (μg) | Mole % |
|---|---|---|
| Arabinose (Ara) | n.d. | n.d. |
| Rhamnose (Rha) | 2.7 | 1.6 |
| Fucose (Fuc) | n.d. | n.d. |
| Xylose (Xyl) | 70.2 | 44.2 |
| Glucuronic acid (GlcA) | n.d. | n.d. |
| Galacturonic acid (GalA) | n.d. | n.d. |
| Mannose (Man) | 3.5 | 1.8 |
| Galactose (Gal) | 65.4 | 34.2 |
| Glucose (Glc) | 34.7 | 18.2 |
| N-acetyl galactosamine (GalNAc) | n.d. | n.d. |
| N-acetyl glucosamine (GlcNAc) | trace | trace |
| | Σ = 176.5 | |

TABLE 11
Porphyridium cruentum monosaccharide analysis

| Glycosyl residue | Mass (μg) | Mole % |
|---|---|---|
| Arabinose (Ara) | n.d. | n.d. |
| Rhamnose (Rha) | n.d. | n.d. |
| Fucose (Fuc) | n.d. | n.d. |
| Xylose (Xyl) | 148.8 | 53.2 |
| Glucuronic Acid (GlcA) | 14.8 | 4.1 |
| Mannose (Man) | n.d. | n.d. |
| Galactose (Gal) | 88.3 | 26.3 |
| Glucose (Glc) | 55.0 | 16.4 |
| N-acetyl glucosamine (GlcNAc) | trace | trace |
| N-acetyl neuraminic acid (NANA) | n.d. | n.d. |
| | Σ = 292.1 | |

Mole % values are expressed as mole percent of total carbohydrate in the sample.
n.d. = none detected.

### Example 5

Porphyridium sp. was grown as described. 2 liters of centrifuged Porphyridium sp. culture supernatant were autoclaved at 121° C. for 20 minutes and then treated with 50% isopropanol to precipitate polysaccharides. Prior to autoclaving the 2 liters of supernatant contained 90.38 mg polysaccharide. The pellet was washed with 20% isopropanol and dried by lyophilization. The dried material was resuspended in deionized water. The resuspended polysaccharide solution was dialyzed to completion against deionized water in a Spectra/Por cellulose ester dialysis membrane (25,000 MWCO). 4.24% of the solid content in the solution was proteins as measured by the BCA assay.

### Example 6

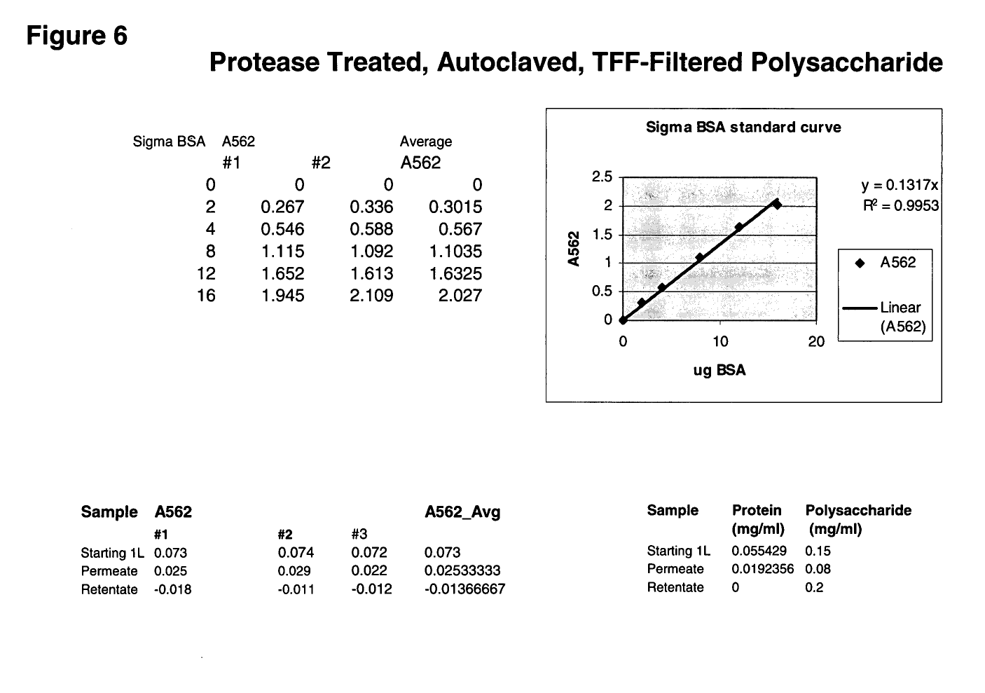

Porphyridium sp. was grown as described. 1 liters of centrifuged Porphyridium sp. culture supernatant was autoclaved at 121° C. for 15 minutes and then treated with 10% protease (Sigma catalog number P-5147; protease treatment amount relative to protein content of the supernatant as determined by BCA assay). The protease reaction proceeded for 4 days at 37° C. The solution was then subjected to tangential flow filtration in a Millipore Pellicon® cassette system using a 0.1 micrometer regenerated cellulose membrane. The retentate was diafiltered to completion with deionized water. No protein was detected in the diafiltered retentate by the BCA assay. See FIG. 6.

Optionally, the retentate can be autoclaved to achieve sterility if the filtration system is not sterile. Optionally the sterile retentate can be mixed with pharmaceutically acceptable carrier(s) and filled in vials for injection.

Optionally, the protein free polysaccharide can be fragmented by, for example, sonication to reduce viscosity for parenteral injection as, for example, an antiviral compound. Preferably the sterile polysaccharide is not fragmented when prepared for use as a lubricant/antiviral compound to be applied topically or with an STD prevention device.

### Example 7

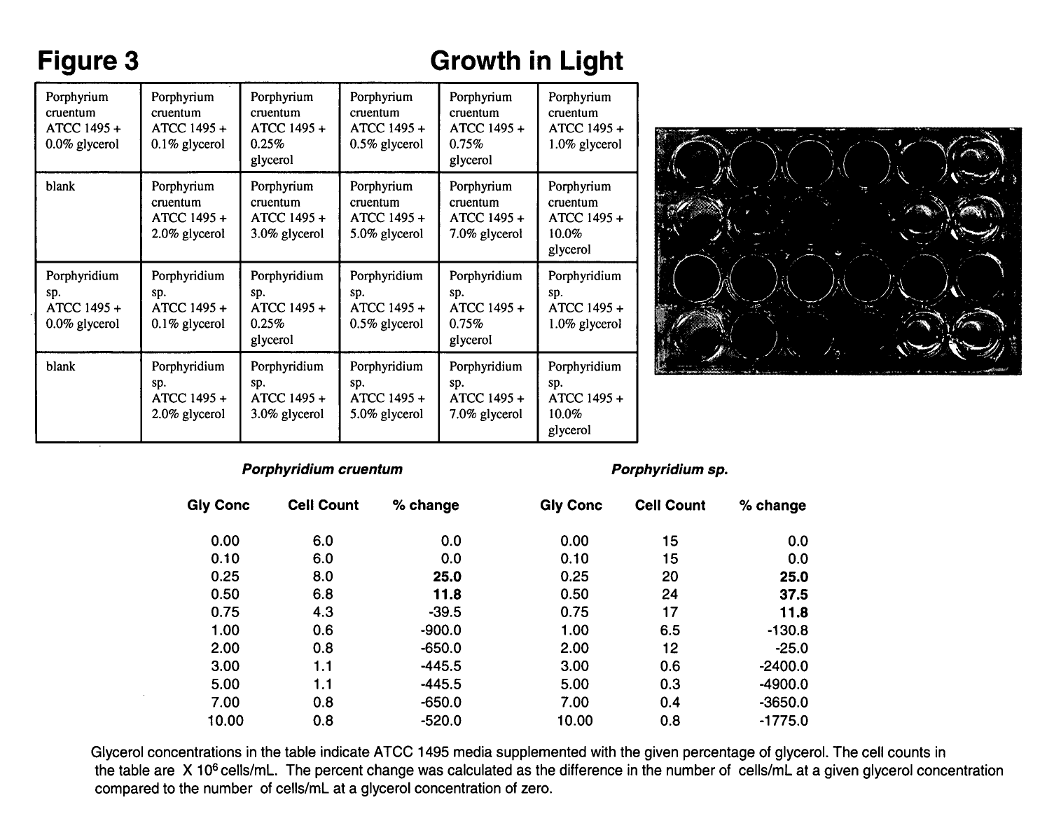

Cultures of Porphyridium sp. (UTEX 637) and Porphyridium cruentum (strain UTEX 161) were grown, to a density of 4×10⁶ cells/mL, as described in Example 1. For each strain, about 2×10⁶ cells/mL cells per well (˜500 uL) were transferred to 11 wells of a 24 well microtiter plate. These wells contained ATCC 1495 media supplemented with varying concentration of glycerol as follows: 0%, 0.1%, 0.25%, 0.5%, 0.75%, 1%, 2%, 3%, 5%, 7% and 10%. Duplicate microtiter plates were shaken (a) under continuous illumination of approximately 2400 lux as measured by a VWR Traceable light meter (cat # 21800-014), and (b) in the absence of light. After 5 days, the effect of increasing concentrations of glycerol on the growth rate of these two species of Porphyridium in the light was monitored using a hemocytometer. The results are given in FIG. 3 and indicate that in light, 0.25 to 0.75 percent glycerol supports the highest growth rate, with an apparent optimum concentration of 0.5%.

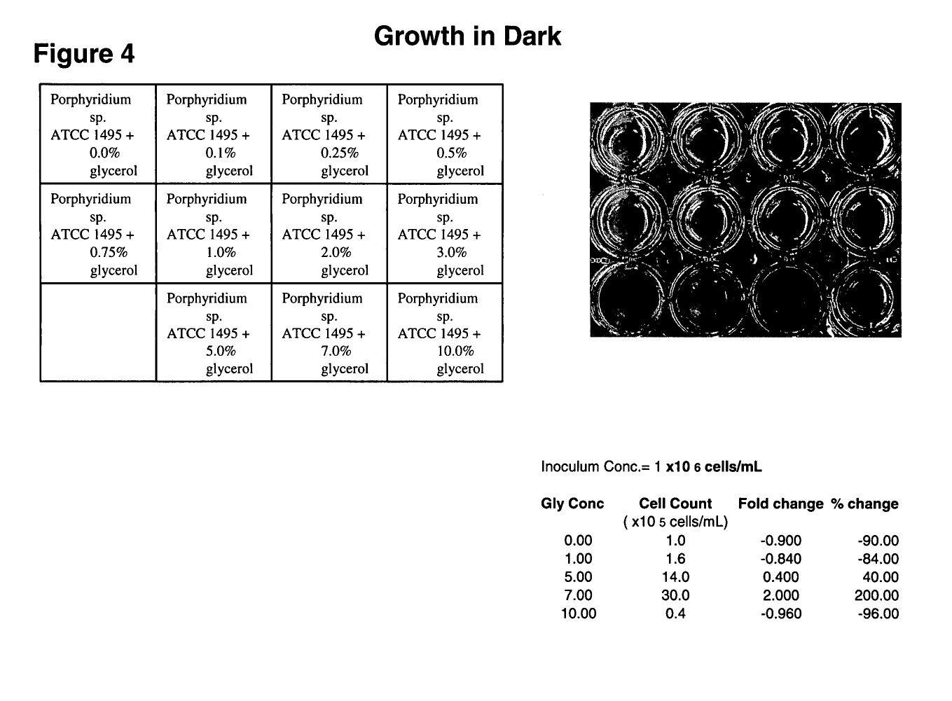

Cells in the dark were observed after about 2 weeks of growth. The results are given in FIG. 4 and indicate that in complete darkness, 5.0 to 7.0% glycerol supports the highest growth rate, with an apparent optimum concentration of 7.0%.

### Example 8

### Sexually Transmitted Disease Prevention Compositions

Polysaccharide from Porphyridium sp. ws prepared as described in Example 2. Lyophilized polysaccharide was resuspended with distilled water to an antivirally effective concentration of 0.5 milligram per mL. 1.0 mL of the 0.5 mg/mL polysaccharide solution was applied to a latex condom.

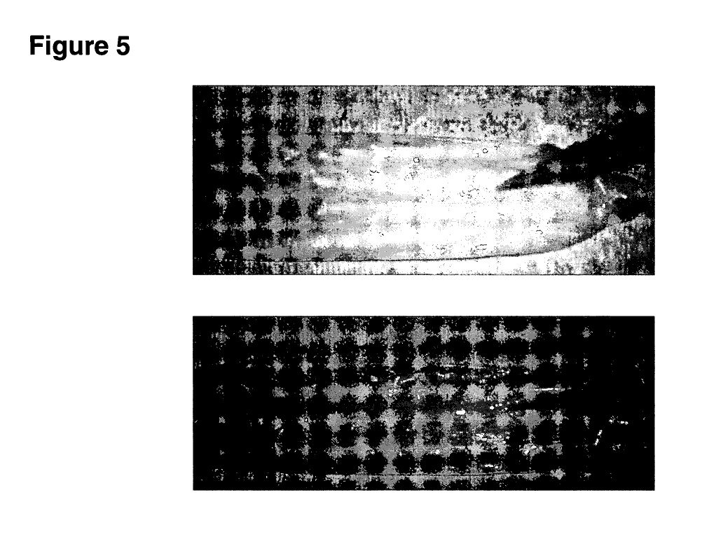

In a second composition formulation, 10 microliters of a 1 mg/mL Porphyridium sp. polysaccharide solution was applied to a latex condom. 29 additional 10 microliter increments of the 1 mg/mL solution were successively applied, creating individual sexually transmitted disease composition with between 100 micrograms and 3 milligrams of polysaccharide in 100 microgram increments. See FIG. 5.

Other condom formulation techniques can be used (see for example U.S. Pat. No. 6,196,227).

### Example 9

Approximately 4500 cells (300 ul of 1.5×10⁵ cells per ml) of Porphyridium sp. and Porphyridium cruentum cultures in liquid ATCC 1495 ASW media were plated onto ATCC 1495 ASW agar plates (1.5% agar). The plates contained varying amounts of zeocin, sulfometuron, hygromycin and spectinomycin. The plates were put under constant artificial fluorescent light of approximately 480 lux. After 14 days, plates were checked for growth. Results were as follows:

| | Growth |
|---|---|
| Zeocin | |
| Conc. (ug/ml) | |
| 0.0 | ++++ |
| 2.5 | + |
| 5.0 | − |
| 7.0 | − |
| Hygromycin | |
| Conc. (ug/ml) | |
| 0.0 | ++++ |
| 5.0 | ++++ |
| 10.0 | ++++ |
| 50.0 | ++++ |
| Specinomycin | |
| Conc. (ug/ml) | |
| 0.0 | ++++ |
| 100.0 | ++++ |
| 250.0 | ++++ |
| 750.0 | ++++ |

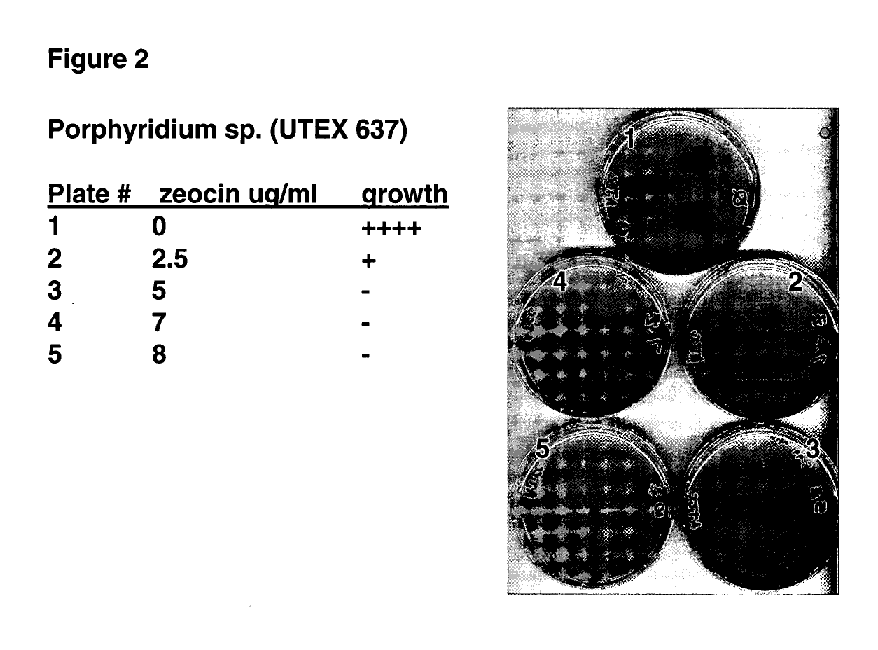

After the initial results above were obtained, a titration of zeocin was performed to more accurately determine growth levels of Porphyridium in the presence of zeocin. Porphyridium sp. cells were plated as described above. Results are shown in FIG. 2.

### Example 10

### Trophic Conversion: Transporters

Cloning

Plasmid pBluescript KS+ is used as a recipient vector for an expression cassette. A promoter active in microalgae is cloned into pBluescript KS+, followed by a 3′ UTR also active in microalgae. Unique restriction sites are left between the promoter and 3′UTR. A nucleic acid encoding a glucose transporter (SEQ ID NO:14) using most preferred codons of Porphyridium sp. is cloned into the unique restriction sites between the promoter and 3′UTR. The promoter:cDNA:3′UTR (SEQ ID NO: 33) is cloned into a plasmid.

The plasmid is used to transform Porphyridium sp. cells using the biolistic transformation parameters described in Plant Physiol. 2002 May; 129(1):7-12. After transformation, some plated cells are scraped from the plate using a sterile cell scraper are transferred into Erlenmeyer flasks wrapped with aluminum foil sufficient to prevent the entry of light into the culture. Identical preparations of transformed, scraped cells are cultured, shaking at ˜50 rpm in 24 well plates in the dark, in ATCC 1495 media in the presence of 0.1, 1.0, and 2.5% glucose, and monitored for growth. Other cells are transformed on plates containing solid agar ATCC 1495 media, supplemented with either 0.1, 1.0, or 2.5% glucose, and monitored for growth in complete darkness.

### Example 11

Cultures of Porphyridium sp. (UTEX 637) and Porphyridium cruentum (strain UTEX 161) were subjected to chemical mutagenesis (from the protocol in Gorman D S, Levine R P. (1965) Proc Natl Acad Sci USA. 54(6):1665-9.). Cells were grown to a density of 4×10⁶ cells/mL as described in Example 1. Cells were harvested, washed with 70 mM potassium phosphate buffer (pH 6.9) and resuspended to a density of 4×10⁷ cells/mL. To 1 mL of cells (from both strains), 0.1M ethyl methane sulfonate (EMS) was added. A 200 uL aliquot was taken for the zero time point. The tubes were incubated in the dark at room temperature. 200 uL aliquots were removed from the tube at various time points: 15 min, 30 min, 45 min and 60 min. At each time, the aliquot of cells were treated with 800 uL of 5% sodium thiosulfate to inactivate the EMS. Cells from each aliquot were spun down and washed three times with 1 mL of 70 mM potassium phosphate buffer (pH 6.9), followed by a wash with 1 mL of ATCC 1495 media. The cells were resuspended in 200 uL of ATCC 1495 media, and plated at three different concentrations (1×, 10⁻²×, 10⁻⁴×) on duplicate plates of ATCC 1495 media, and incubated under continuous light.

After mutagenesis, some plated cells are scraped from the plate using a sterile cell scraper are transferred into Erlenmeyer flasks wrapped with aluminum foil sufficient to prevent the entry of light into the culture. Identical preparations of transformed, scraped cells are cultured, shaking at ˜50 rpm in 24 well plates in the dark, in ATCC 1495 media in the presence of 0.1, 1.0, and 2.5% glucose, and monitored for growth. Other cells are transformed on plates containing solid agar ATCC 1495 media, with either 0.1, 1.0, or 2.5% glucose, and monitored for growth in complete darkness. Cell treated as described can also be cultured in the presence of an exogenous carbon source from Tables 2 or 3.

### Example 12

### Genetic and Nutritional Manipulation to Generate Novel Polysaccharides

Cells prepared as described in Example 10, containing a monosaccharide transporter and capable of importing glucose, are cultured in ATCC 1495 media in the light in the presence of 1.0% glucose for approximately 12 days. Exopolysaccharide is purified as described in Example 2. Monosaccharide analysis is performed as described in Example 4.

Cells prepared as described in Example 10, containing a monosaccharide transporter and capable of importing xylose, are cultured in ATCC 1495 media in the light in the presence of 1.0% xylose for approximately 12 days. Exopolysaccharide is purified as described in Example 2. Monosaccharide analysis is performed as described in Example 4.

Cells prepared as described in Example 10, containing a monosaccharide transporter and capable of importing galactose, are cultured in ATCC 1495 media in the light in the presence of 1.0% galactose for approximately 12 days. Exopolysaccharide is purified as described in Example 2. Monosaccharide analysis is performed as described in Example 4.

Cells prepared as described in Example 10, containing a monosaccharide transporter and capable of importing glucuronic acid, are cultured in ATCC 1495 media in the light in the presence of 1.0% glucuronic acid for approximately 12 days. Exopolysaccharide is purified as described in Example 2. Monosaccharide analysis is performed as described in Example 4.

Cells prepared as described in Example 10, containing a monosaccharide transporter and capable of importing glucose, are cultured in ATCC 1495 media in the dark in the presence of 1.0% glucose for approximately 12 days. Exopolysaccharide is purified as described in Example 2. Monosaccharide analysis is performed as described in Example 4.

Cells prepared as described in Example 10, containing a monosaccharide transporter and capable of importing xylose, are cultured in ATCC 1495 media in the dark in the presence of 1.0% xylose for approximately 12 days. Exopolysaccharide is purified as described in Example 2. Monosaccharide analysis is performed as described in Example 4.

Cells prepared as described in Example 10, containing a monosaccharide transporter and capable of importing galactose, are cultured in ATCC 1495 media in the dark in the presence of 1.0% galactose for approximately 12 days. Exopolysaccharide is purified as described in Example 2. Monosaccharide analysis is performed as described in Example 4.

Cells prepared as described in Example 10, containing a monosaccharide transporter and capable of importing glucuronic acid, are cultured in ATCC 1495 media in the dark in the presence of 1.0% glucuronic acid for approximately 12 days. Exopolysaccharide is purified as described in Example 2. Monosaccharide analysis is performed as described in Example 4.

### Example 13

Porphyridium cruentum was grown as described above in ATCC 1495 media. Porphyridium cruentum culture supernatant were autoclaved at 121° C. for 20 minutes. 1.333 liters of isopropanol was added to a 4 liter preparation of autoclaved supernatant to a concentration of 25% (vol/vol). Precipitated exopolysaccharide was removed. Additional isopropanol (381 mL, 786 mL, 167 mL, and 1.333 liters) was added stepwise to the preparation to produce (vol/vol) concentrations of isopropanol of 30%, 38.5%, 40%, and 50%, respectively. Precipitated exopolysaccharide was removed after each increment of isopropanol was added. It was observed that very little additional exopolysaccharide was precipitated upon bringing the concentration from 38.5% to 40% and from 40% to 50%. It was also observed that significant amounts of salt were precipitated upon bringing the concentration from 38.5% to 40% and from 40% to 50%.

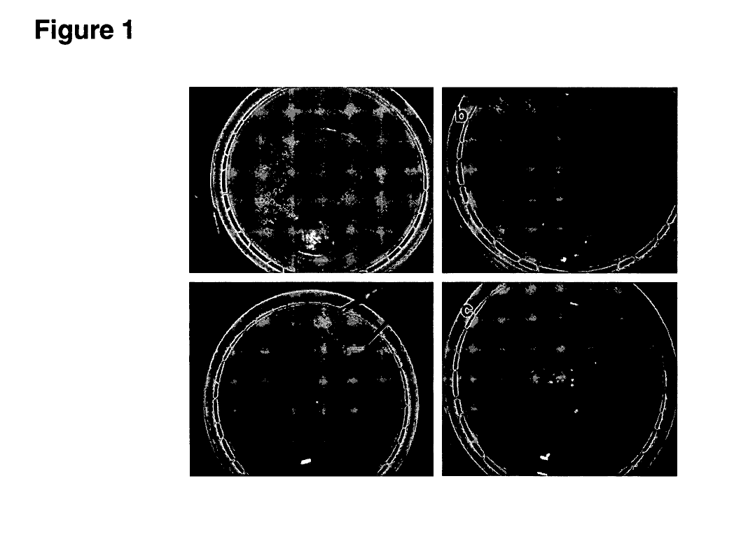

An additional 4 liters of exopolysaccharide was precipitated with by addition of 38.5% isopropanol. See FIG. 1.

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1-23. (canceled)

24. A sexually transmitted disease prevention kit or composition comprising:
a. a solution comprising a polysaccharide produced from microalgae; and
b. a prophylactic device.

25. The kit or composition of claim 24, wherein the device is a condom that is packaged in direct contact with the solution.

26. The kit or composition of claim 24, wherein the composition lacks a lubricant other than polysaccharide.

27. The kit or composition of claim 24, wherein the polysaccharide provides both lubricant function and antiviral activity.

28. The kit or composition of claim 24, wherein the polysaccharide is sulfated.

29. The kit or composition of claim 24, wherein the microalgae is selected from Table 1.

30. The kit or composition of claim 25, wherein the microalgae is selected from the group consisting of Porphyridium sp. and Porphyridium cruentum.

31. The kit or composition of claim 24, wherein the polysaccharide is associated with a fusion protein comprising a first protein with at least 60% amino acid identity with the protein of SEQ ID NO: 21, and a second protein.

32. The kit or composition of claim 31, wherein the second protein is an antibody that selectively binds to an antigen from a pathogen selected from the group consisting of HIV, Herpes Simplex Virus, gonorrhea, Chlamydia, Human Papillomavirus, and Trichomoniasis.

33-40. (canceled)

41. A method of treating or effecting prophylaxis of a mammal having or at risk of a viral infection comprising administering a polysaccharide produced by microalgae to the mammal and thereby treating or effecting prophylaxis of the mammal.

42. The method of claim 41, wherein the administration is mucosal.

43. The method of claim 41, wherein the administration is parenteral.

44. The method of claim 43, wherein the average molecular weight of the polysaccharide is about 100,000 daltons.

45. The method of claim 43, wherein the average molecular weight of the polysaccharide is about 50,000 daltons.

46. The method of claim 41, wherein the polysaccharide is produced by a microalgae listed in Table 1.

47. The method of claim 46, wherein the microalgae is of the genus Porphyridium.

48-63. (canceled)

64. A method of purifying an exopolysaccharide from cultures of cells of red microalgae comprising:
a. culturing red microalgae cells;
b. separating cells from culture media;
c. adding isopropanol to the culture media; and
d. separating precipitated exopolysaccharide from the solution.

65-66. (canceled)

67. The method of claim 64, wherein the concentration of isopropanol used to precipitate the exopolysaccharide is between about 36% and 40% vol/vol.

68. The method of claim 64, wherein the concentration of isopropanol used to precipitate the exopolysaccharide is about 38.5% vol/vol.

69. The method of claim 64, wherein the red microalgae cells ore of the genus Porphyridium.

70. (canceled)
